(19) United States
(12) Patent Application Publication
(10) Pub. No.: US 2018/0022780 A1
(43) Pub. Date: Jan. 25, 2018

(54) STABILIZATION ADRENOMEDULLIN DERIVATIVES AND USE THEREOF

(71) Applicant: BAYER PHARMA AKTIENGESELLSCHAFT, Berlin (DE)

(72) Inventors: Donald BIERER, Haan (DE); Ingo FLAMME, Reichshof (DE); Johannes KÖBBERLING, Neuss (DE); Bernd RIEDL, Wuppertal (DE); Annette BECK-SICKINGER, Leipzig (DE); Ria SCHOENAUER, Leipzig (DE); Jan-Patrick FISCHER, Leipzig (DE)

(21) Appl. No.: 15/514,456

(22) PCT Filed: Sep. 24, 2015
(86) PCT No.: PCT/EP2015/071941

§ 371 Date: Mar. 24, 2017

(30) Foreign Application Priority Data
Sep. 26, 2014 (EP) 14186572.5

(51) Int. Cl. C07K 14/00; A61K 38/16; A61K 38/55

(52) U.S. Cl. CPC C07K 14/001; A61K 38/55; A61K 38/16; C07K 2319/30; C07K 2319/31; A61K 38/00

(57) ABSTRACT

The invention relates to novel, biologically active, stabilized Adrenomedullin (ADM) compounds. The invention further relates to the compounds for use in a method for the treatment and/or prevention of diseases, especially of cardiovascular, edematous and/or inflammatory disorders, and to medicaments comprising the compounds for treatment and/or prevention of cardiovascular, edematous and/or inflammatory disorders.

The present invention relates to novel, biologically active, stabilized Adrenomedullin (ADM) peptide derivatives. The compounds of the invention are stabilized by substitution of the intramolecular disulfide bond and optionally one or more further modifications selected from replacement of amino acids by natural or unnatural amino acids, covalently linking the peptide derivative to a heterologous moiety selected from the group consisting of a polymer, a Fc, a FcRn binding ligand, albumin and an albumin-binding ligand, and N-methylation of at least one amide bond. The invention further relates to the compounds for use in a method for the treatment and/or prevention of diseases, especially of cardiovascular, edematous and/or inflammatory disorders, and to medicaments comprising the compounds for treatment and/or prevention of cardiovascular, edematous and/or inflammatory disorders.

The 52 amino acid peptide hormone adrenomedullin (ADM) is produced in adrenal gland, lung, kidney, heart muscle and other organs. The plasma levels of ADM are in the lower picomolar range. ADM is a member of the calcitonin gene-related peptide (CGRP) family of peptides and as such binds to a heterodimeric G-protein coupled receptor that consists of CRLR and RAMP 2 or 3 (Calcitonin-receptor-like receptor and receptor activity modifying protein 2 or 3). Activation of the ADM receptor leads to intracellular elevation of adenosine 3′, 5′-cyclic monophosphate (cAMP) in the receptor-bearing cells. ADM receptors are present on different cell types in almost all organs including endothelial cells. ADM is thought to be metabolized by neutral endopeptidase and is predominantly cleared in the lung where ADM-receptors are highly expressed [for review see Gibbons C, Dackor R, Dunworth W, Fritz-Six K, Caron K M, Mol Endocrinol 21(4), 783-796 (2007)].

Experimental data from the literature suggest that ADM is involved in a variety of functional roles that include, among others, blood pressure regulation, bronchodilatation, renal function, hormone secretion, cell growth, differentiation, neurotransmission, and modulation of the immune response. Moreover ADM plays a crucial role as autocrine factor during proliferation and regeneration of endothelial cells [for review see García M. A., Martín-Santamaría S., de Pascual-Teresa B., Ramos A., Julián M., Martínez A., Expert Opin Ther Targets, 10(2), 303-317 (2006)].

There is an extensive body of evidence from the literature which shows that ADM is indispensable for an intact endothelial barrier function and that administration of ADM to supra-physiological levels exerts strong anti-edematous and anti-inflammatory functions in a variety of inflammatory conditions in animal experiments including sepsis, acute lung injury and inflammation of the intestine [for review see Temmesfeld-Wollbriick B, Hocke A., Suttorp N, Hippenstiel S, Thromb Haemost; 98, 944-951 (2007)].

Clinical testing of ADM was so far conducted in cardiovascular indications with a measurable hemodynamic end point such as pulmonary hypertension, hypertension, heart failure and acute myocardial infarction. ADM showed hemodynamic effects in several studies in patients suffering from the aforementioned conditions. However, effects were only short lasting and immediately ceasing after the end of administration. This findings correlated well with the known pharmacokinetic profile of ADM. Pharmacodynamic effects comprised among others lowering of systemic and pulmonary arterial blood pressure and increase of cardiac output [Troughton R W, Lewis L K, Yandle T G, Richards A M, Nicholls M G, Hypertension, 36(4), 588-93 (2000); Nagaya N, Kangawa K, Peptides, 25(11), 2013-8 (2004); Kataoka Y, Miyazaki S, Yasuda S, Nagaya N, Noguchi T, Yamada N, Morii I., Kawamura A, Doi K, Miyatake K, Tomoike H, Kangawa K, J Cardiovasc Pharmacol, 56(4), 413-9 (2010)].

In summary, based on evidence from a wealth of experimental data in animals and first clinical trials in man elevation of ADM to supraphysiological levels might be considered as a target mechanism for the treatment of a variety of disease conditions in man and animals. However, the major limitations of the use of ADM as therapeutic agent are the inconvenient applicability of continuous infusion therapy which precludes its use for most of the potential indications and the potentially limited safety margins with respect to hypotension which may result from bolus administrations of ADM.

The object of the present invention is to provide novel biologically active, stabilized ADM peptide derivatives which can be employed for the treatment of diseases, in particular cardiovascular, edematous and inflammatory disorders.

Many therapeutically active peptides or proteins suffer from high clearance in vivo. Several approaches to increase the stability of therapeutically active peptides or proteins and reduce their clearance exist, including the alteration of disulfide bonds, N-methylation of amide bonds, and conjugation with heterologous moieties such as polymers and proteins.

Peptide therapeutics containing disulfide bonds may be problematic in their application in vivo. Disulfide bridges are unstable towards reducing agents and disulfide isomerases. Reduction of the disulfide bond results in a structural rearrangement and in a loss of activity. Protein-disulfide isomerase (PDI) is an enzyme of the endoplasmatic reticulum. Protein folding pathways contain intermediates with non-native disulfide bridges. The essential PDI function is to rearrange these intermediates to reach the final conformation [Laboissiere M C, Sturley S L, Raines R T, The essential function of protein-disulfide isomerase is to unscramble non-native disulfide bonds, J Biol Chem., 270(47), 28006-28009, 1995]. Glutathione (GSH) reacts with somatostatin to form mixed disulfides, further reaction with a second GSH molecule leads to the reduced dithiol form of somatostatin and GSSG. Thiol/disulfide exchange occurs readily; however, the formed mixed disulfides rapidly undergo reformation of the intramolecular disulfide bonds [Rabenstein D L, Weaver K H, Kinetics and equilibria of the thiol/disulfide exchange reactions of somatostatin with glutathione, J Org Chem., 61(21), 7391-7397, 1996]. The role of disulfide bonds in structural stability of peptides is described in Gehrmann J, Alewood P F, Craik D J, Structure determination of the three disulfide bond isomers of α-conotoxin GI: a model for the role of disulfide bonds in structural stability, J Mol Biol., 278(2), 401-415, 1998.

Cystathiones are resistant towards thiol reduction. Therefore, substitutions of disulfides with thioethers are interesting in drug discovery, as they provide protection against reduction while the structure is only minimally perturbed. Thioether analogues of the complement inhibitor peptide compstatin were synthesized. The inhibitory potential was largely retained, whereas the stability to reduction was improved [Knerr P J, Tzekou A, Ricklin D, Qu H, Chen H, van der Donk W A, Lambris J D, Synthesis and activity of thioether-containing analogues of the complement inhibitor compstatin, ACS Chem Biol., 6(7), 753-760, 2011]. Peptide disulfide bond mimics based on diaminodiacids are described e.g. in Cui H K, Guo Y, He Y, Wang F L, Chang H N, Wang Y J, Wu F M, Tian C L, Liu L, Diaminodiacid-based solid-phase synthesis of peptide disulfide bond mimics, Angew Chem, 125, 9737-9741, 2013. Thioether and biscarba diaminodiacids were applied in the synthesis of peptide disulfide bond mimics of tachyplesin I analogues. The derivatives exhibited a decreased antimicrobial activity, but improved serum stability.

Kowalczyk R, Harris P W, Brimble M A, Callon K E, Watson M, Cornish J, Synthesis and evaluation of disulfide bond mimetics of amylin-(1-8) as agents to treat osteoporosis, Bioorg Med Chem., 20(8), 2661-2668, 2012, pertains to the octapeptide amylin. The native peptide (1-8) is stable for 6 month only at −80° C. under argon atmosphere. Analogues of the peptide were synthesized, wherein the disulfide bridge was modified either by the insertion of linkers or bridges of a different nature. All analogues were bench stable and therefore exhibited an improved stability. Muttenthaler M, Andersson A, de Araujo A D, Dekan Z, Lewis R J, Alewood P F, Modulating oxytocin activity and plasma stability by disulfide bond engineering, J Med Chem., 53(24), 8585-8596, 2010, pertains to the synthesis of oxytocin analogues with disulfide bond replacements (thioether, selenosulfide, diselenide and ditelluride bridges) in order to improve the metabolic half-life of cysteine-containing peptides. Compared to oxytocin, some analogues retained affinity and functional potency and all mimetics exhibited an increase (1.5-3-fold) in plasma stability. Pakkala M, Weisell J, Hekim C, Vepsäläinen J, Wallen E A, Stenman U H, Koistinen H, Narvanen A, Mimetics of the disulfide bridge between the N- and C-terminal cysteines of the KLK3-stimulating peptide B-2, Amino Acids., 39(1), 233-242, 2010, pertains to kallikrein-related peptidase 3 (KLK3). The proteolytic activity of kallikrein-related peptidase 3 (KLK3) is promoted by the synthetic cyclic, disulfide-bridged peptide B-2. Replacement of the disulfide with a lactam bridge between γ-butyric acid and aspartic acid was performed. The resulting peptide had an improved stability in plasma and against degradation by KLK3, as well as a higher activity than B-2 at high concentrations. Watkins H A, Rathbone D L, Barwell J, Hay D L, Poyner D R, Structure-activity relationships for α-calcitonin gene-related peptide, Br J Pharmacol., 170(7), 1308-1322, 2013, summarizes SAR studies performed with the α-calcitonin gene-related peptide (CGRP), the closest analogue of adrenomedullin. Referred is a disulfide mimic with a lactam as substitute (cyclo [Asp², Lys⁷]-CGRP), which is originally described in: Dennis T, Fournier A, St Pierre S, Quirion R, Structure-activity profile of calcitonin gene-related peptide in peripheral and brain tissues. Evidence for receptor multiplicity. J Pharmacol Exp Ther., 251(2), 718-725, 1989. This peptide showed 50% decrease in affinity to the receptor in rat spleen membranes. Measurements of biological activity in guinea pig atria indicate a loss of agonist function.

Further, several approaches to form an injectable depot of such drugs exist that involve the use of macromolecules.

Polymer matrices that contain a drug molecule in a non covalently bound state are well known. These can also be injectable as hydro gels, micro particles or micelles. The release kinetics of such drug products can be quite unreliable with high inter patient variability. Production of such polymers can harm the sensitive drug substance or it can undergo side reactions with the polymer during its degradation [D. H. Lee et al., J. Contr. Rd., 92, 291-299, 2003].

Permanent PEGylation of peptides or proteins to enhance their solubility, reduce immunogenicity and increase half live by reducing renal clearance is a well known concept since early 1980s [Caliceti P., Veronese F. M., Adv. Drug Deliv. Rev., 55, 1261-1277, 2003]. For several drugs this has been used with success, but with many examples the PEGylation reduces efficacy of drug substance to an extent that this concept is not suitable any more [T. Peleg-Shulman et al., J. Med. Chem., 47, 4897-4904, 2004].

A suitable alternative are polymer based prodrugs. The current definitions for prodrugs by the IUPAC state the following terms [International Union of Pure and Applied Chemistry and International Union of Biochemistry: GLOSSARY OF TERMS USED IN MEDICINAL CHEMISTRY (Recommendations 1998); in Pure & Appl. Chem. Vol 70, No. 5, p. 1129-1143, 1998]:

Prodrug: A prodrug is any compound that undergoes biotransformation before exhibiting its pharmacological effects. Prodrugs can thus be viewed as drugs containing specialized non-toxic protective groups used in a transient manner to alter or to eliminate undesirable properties in the parent molecule.

Carrier-linked prodrug (Carrier prodrug): A carrier-linked prodrug is a prodrug that contains a temporary linkage of a given active substance with a transient carrier group that produces improved physicochemical or pharmacokinetic properties and that can be easily removed in vivo, usually by a hydrolytic cleavage.

Cascade prodrug: A cascade prodrug is a prodrug for which the cleavage of the carrier group becomes effective only after unmasking an activating group.

Several examples of PEG-based carrier prodrugs exist, most of them with the need for enzymatic activation of the linker between the active drug and the carrier, mostly initiated by enzymatic hydrolysis.

Since esters are cleaved very readily and unpredictably in vivo, direct ester linkers for carrier pro drug have limitations to their usability [J. Rautio et al., Nature Reviews Drug discovery, 7, 255-270, 2008].

Commonly used alternative approaches are cascading linkers attached to an amine functionality in the peptide or protein. In cascading linkers a masking group has to be removed as the rate limiting step in the cascade. This activates the linker to decompose in a second position to release the peptide or protein. Commonly the masking group can be removed by an enzymatic mechanism [R. B. Greenwald et al. in WO 2002/089789, Greenwald, et al., J. Med. Chem. 1999, 42, 3657-3667, F. M. H. DeGroot et al. in WO 2002/083180 and WO 2004/043493, and D. Shabat et al. in WO 2004/019993].

An alternative not relying on enzymatic activation is the concept of U. Hersel et al. in WO 2005/099768. In their approach the masking group on a phenol is removed in a purely pH dependent manner by the attack of an internal nucleophile. This activates the linker for further decomposition.

As mentioned by U. Hersel et al. in WO 2005/099768, “The disadvantage in the abovementioned prodrug systems described by Greenwald, DeGroot and Shabat is the release of potentially toxic aromatic small molecule side products like quinone methides after cleavage of the temporary linkage. The potentially toxic entities are released in a 1:1 stoichiometry with the drug and can assume high in vivo concentrations.” The same problem holds true for the system by Hersel et al. as well.

For small organic molecules a plethora of different prodrug approaches exist [J. Rautio et al., Nature Reviews Drug discovery, 7, 255-270, 2008]. The approach used by U. Hersel et al. as release mechanism for their masking group has been used as a prodrug approach for phenolic groups of small molecules since the late 1980s. [W. S. Saari in EP 0 296 811 and W. S. Saari et al., J. Med. Chem., Vol 33, No 1, p 97-101, 1990].

Alternative amine based prodrug systems are based on the slow hydrolysis of bis-hydroxyethyl glycine as a cascading prodrug. The hydroxy groups of the bis-hydroxyethyl glycine are masked by esters that are prone to hydrolysis by esterases [R. Greenwald et al., J. Med. Chem., 47, 726-734, 2004, and D. Vetter et al. in WO 2006/136586].

Purely pH dependent cleavage of linkers is more reliable then enzymatic cleavage of linkers as it is not dependent on enzyme concentrations that may vary in living systems.

One concept for linkers that are cleaved pH dependently are prodrugs based on beta elimination with adjustable decomposition rates as described by Santi et al. in U.S. Pat. No. 8,680,315. The described linker technology to reversibly attach macromolecules to peptides and small molecules is applicable to several functional groups in the released drug. Amines, alcohols, carboxylic acids and thiols are attachable via an adaptor system to the beta eliminating moiety. Upon pH triggered decomposition the drug is released upon release of CO₂ and an unsaturated fragment attached to the macromolecule.

Another approach optimized for phenols namely tyrosine in peptides is based on a carbamate that is pH dependently attacked by a nucleophilic amine under release of the phenol and generation of a cyclic urea attached to the macromolecule as described by Flamme I. et al in WO 2013/064455.

Further heterologous moieties established for the adjustment of the pharmacokinetic properties of peptides include polymers, including linear or branched C₃-C₁₀₀ carboxylic acids (lipidation), a polyethyleneglycol (PEG) moiety, a polypropylenglycol (PPG) moiety, a PAS moiety, which is an amino acid sequence comprising mainly alanine and serine residues or comprising mainly alanine, serine, and proline residues, the amino acid sequence forming random coil conformation under physiological conditions [US No. 2010/0292130 and WO 2008/155134], and a hydroxyethylstarch (HES) moiety [WO 02/080979], a Fc, a FcRn binding ligand, albumin and an albumin-binding ligand.

The adjustment of the pharmacokinetic properties of peptides by lipidation is a well-developed methodology. Lipidation can occur to the N-terminus or to the side chain functionalities of amino acids within the peptide sequence. Lipidation is described in a plethora of publications and patents as exemplified in the following reviews: Zhang L, Bulaj G, Converting peptides into drug leads by lipidation, Curr Med Chem.; 19(11):1602-18, 2012, or M. Gerauer, S. Koch, H. Waldmann, L. Brunsveld, Lipidated peptide synthesis: Wiley Encyclopedia of Chemical Biology, Volume 2, 520-530, 2009, (Hrsg. Begley, T. P.). John Wiley & Sons, Hoboken, N.J. The lipidation of a truncated ADM fragment is described in WO 2012/138867.

Labeled Adrenomedullin derivatives for use as imaging and also therapeutic agent are known [J. Depuis et al. in CA 2567478 and WO 2008/138141]. In these ADM derivatives a complexating cage like molecular structure capable of binding radioactive isotopes was attached to the N terminus of ADM in a direct manner or via a spacer unit potentially also including short PEG spacers. The diagnostic or therapeutic value of theses drugs arises from the targeted delivery of the radioactive molecule.

In contrast to the prodrug approaches listed above, which are all based on masking amine functionalities, another approach described in WO 2013/064508 is based on masking the phenolic group of a tyrosine in ADM. A carrier-linked prodrug is used, based on the internal nucleophile assisted cleavage of a carbamate on this phenolic group. The key advantage to other prodrug classes mentioned above is the toxicological harmlessness of the linker decomposition product, a cyclic urea permanently attached to the carrier. Furthermore, the decomposition of the prodrug is not dependent on enzymatic mechanisms that might cause a high inter patient variability of cleavage kinetics. The cleavage mechanism is solely pH dependent as an internal amine that is protonated at acidic pH gets activated at higher (neutral) pH to act as a nucleophile attacking the phenolic carbamate based on the tyrosine.

In the context of the present invention, stabilized, biologically active ADM peptide derivatives are now described wherein the disulfide bridging of the ADM peptide derivatives was replaced. Optionally, these modified ADM peptide derivatives were further modified by N-Methylation or by covalently linking the peptide derivative to a heterologous moiety selected from the group consisting of a polymer, an Fc, an FcRn binding ligand, albumin and an albumin-binding ligand. The polymer that is covalently linked to the peptide derivative is selected from the group consisting of optionally substituted, saturated, or mono- or di-unsaturated, linear or branched C₃-C₁₀₀ carboxylic acids, preferably C₄-C₃₀ carboxylic acids, a PEG moiety, a PPG moiety, a PAS moiety and a HES moiety. The analogues were investigated by means of activity and stability. It was shown that the activity of the ADM derivatives is retained as compared to wt ADM. Further, the stabilized ADM peptide derivatives show an increased half-life in blood and liver, as can be shown by stability assays in serum and liver homogenates. The stabilized ADM peptides show extended duration of pharmacological action as compared to ADM and on the basis of this specific action mechanism—after parenteral administration—exert in vivo sustained anti-inflammatory and hemodynamic effects such as stabilization of endothelial barrier function, and reduction of blood pressure, respectively.

The present invention provides compounds of formula (I)

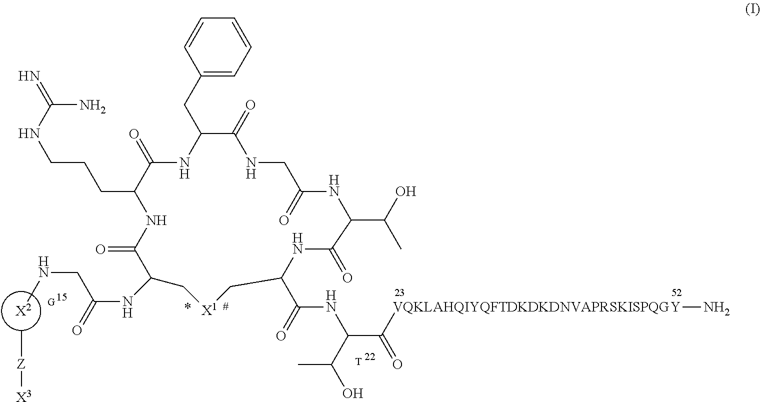

wherein X¹ is selected from the group consisting of
*—(CH₂)ₘ₁—S—^{#}, wherein m1 is 0-6; ^{#}—(CH₂)ₘ₂—S—*, wherein m2 is 0-6;
*—(CH₂)ₘ₃—^{#}, wherein m3 is 1-8;
*—(CH₂)ₘ₄—(CH₂═CH₂)—(CH₂)ₙ₁—^{#}, wherein m4 is 0-6, n1 is 0-6, with the proviso that m4+n1=0-6;
*—(CH₂)ₘ₅—(CH≡CH)—(CH₂)ₙ₂—^{#}, wherein m5 is 0-6, and n2 is 0-6, with the proviso that m5+n2=0-6;
*—(CH₂)ₘ₆—CO—NH—(CH₂)ₙ₃—^{#}, wherein m6 is 0-4, and n3 is 0-4, with the proviso that m6+n3=0-6; ^{#}—(CH₂)ₘ₇—CO—NH—(CH₂)ₙ₄—*, wherein m7 is 0-4, and n4 is 0-4, with the proviso that m7+n4=0-6;
*—SO—(CH₂)ₘ₈—^{#}, wherein m8 is 0-6; ^{#}—SO—(CH₂)ₘ₉—*, wherein m9 is 0-6;
*—SO₂—(CH₂)ₘ₁₀—^{#}, wherein m10 is 0-6; ^{#}—SO₂—(CH₂)ₘ₁₁*, wherein m11 is 0-6;
*-5-6 membered heteroaryl-^{#};
*—O—(CH₂)ₘ₁₂—^{#}, wherein m12 is 0-6; ^{#}—O—(CH₂)ₘ₁₃—*, wherein m13 is 0-6;
*—CH₂—S—(CH₂)ₘ₁₄—^{#}, wherein m14 is 0-6; ^{#}—CH₂—S—(CH₂)ₘ₁₅—*, wherein m15 is 0-6;
*—CH₂—O—(CH₂)ₘ₁₆—^{#}, wherein m16 is 0-6; ^{#}—CH₂—O—(CH₂)ₘ₁₇—*, wherein m17 is 0-6;
*—(CH₂)ₘ₁₈—NH—CO—CH₂—NH—CO—(CH₂)ₙ₅—^{#}, wherein m18 is 0-3, and n5 is 0 or 1, with the proviso that m18+n5=0-3; ^{#}—(CH₂)ₘ₁₉—NH—CO—CH₂—NH—CO—(CH₂)ₙ₆—*, wherein m19 is 0-3, and n6 is 0 or 1, with the proviso that m19+n6=0-3;
*—(CH₂)ₘ₂₀—NH—CO—CH(CH₃)—NH—CO—(CH₂)ₙ₇—^{#}, wherein m20 is 0-3, and n7 is 0 or 1, with the proviso that m20+n7=0-3; ^{#}—(CH₂)ₘ₂₁—NH—CO—CH(CH₃)—NH—CO—(CH₂)ₙ₈—* wherein m21 is 0-3, and n8 is 0 or 1, with the proviso that m21+n8=0-3;
*—(CH₂)ₘ₂₂—NH—CO—CH(CH₂—C(CH₃)₂)—NH—CO—(CH₂)ₙ₉—^{#}, wherein m22 is 0-3, and n9 is 0 or 1, with the proviso that m22+n9=0-3; ^{#}—(CH₂)ₘ₂₃—NH—CO—CH(CH₂—C(CH₃)₂)—NH—CO—(CH₂)ₙ₁₀—*, wherein m23 is 0-3, and n10 is 0 or 1, with the proviso that m23+n10=0-3;
*—(CH₂)ₘ₂₄—NH—CO—CH(CH₃)C₂H₅)—NH—CO—(CH₂)ₙ₁₁—^{#}, wherein m24 is 0-3, and n11 is 0 or 1, with the proviso that m24+n11=0-3; ^{#}—(CH₂)ₘ₂₅—NH—CO—CH(CH(CH₃)C₂H)—NH—CO—(CH₂)ₙ₁₂—*, wherein m25 is 0-3, and n12 is 0 or 1, with the proviso that m25+n12=0-3;
*—(CH₂)ₘ₂₆—NH—CO—CH(CH₂(C₆H₅))—NH—CO—(CH₂)ⁿ—^{#}, wherein m26 is 0-3, and n13 is 0 or 1, with the proviso that m26+n13=0-3; ^{#}—(CH₂)ₘ₂₇—NH—CO—CH(CH₂(C₆H₅))—NH—CO—(CH₂)ₙ₁₄—*, wherein m27 is 0-3, and n14 is 0 or 1, with the proviso that m27+n14=0-3;
*—(CH₂)ₘ₂₈—NH—CO—(CH₂)₃—NH—CO—(CH₂)ₙ₁₅—^{#}, wherein m28 is 0 or 1, and n15 is 0 or 1, with the proviso that m28+n15=0-1; ^{#}—(CH₂)ₘ₂₉—NH—CO—(CH₂)₃—NH—CO—(CH₂)ₙ₁₆—*, wherein m29 is 0 or 1, and n16 is 0 or 1, with the proviso that m29+n16=0-1;
*—(CH₂)ₘ₃₀—NH—CO—NH—(CH₂)ₙ₁₇—^{#}, wherein m30 is 0-5, and n17 is 0-5, with the proviso that m30+n17=0-5; ^{#}—(CH₂)ₘ₃₁—NH—CO—NH—(CH₂)ₙ₁₈—*, wherein m31 is 0-5, and n18 is 0-5, with the proviso that m31+n18=0-5;
*—(CH₂)ₘ₃₂—O—CO—NH—(CH₂)ₙ₁₉—^{#}, wherein m32 is 0-5, and n19 is 0-5, with the proviso that m32+n19=0-5; ^{#}—(CH₂)ₘ₃₃—O—CO—NH—(CH₂)ₙ₂₀—*, wherein m33 is 0-5, and n20 is 0-5, with the proviso that m33+n20=0-5;
*—(CH₂)ₘ₃₄—O—CO—O—(CH₂)ₙ₂₁—^{#}, wherein m 34 is 0-5, and n21 is 0-5, with the proviso that m34+n21=0-5;
*—(CH₂)ₘ₃₅—NH—CO—(CH₂)ₙ₂₂—NH—(CH₂)ₚ₁—, wherein m35 is 0-4, n22 is 0-4, and p1 is 0-4, with the proviso that m35+n22+p1=0-4; and
*—(CH₂)ₘ₃₆—NH—CO—(CH═CH)—CO—NH—(CH₂)ₙ₂₃—^{#}, wherein m36 is 0-2, and n23 is 0-2, with the proviso that m36+n23=0-2;
wherein * and ^{#} reflect where X¹ is bound within the ring structure;
X² is absent, is hydrogen, or is an amino acid or amino acid sequence selected from the group consisting of G¹⁴, K¹⁴, F¹⁴, SEQ ID NO:1 [Y¹RQSMNNFQGLRSF¹⁴], SEQ ID NO:2 [R²QSMNNFQGLRSF¹⁴], SEQ ID NO:3 [Q³SMNNFQGLRSF¹⁴], SEQ ID NO:4 [S⁴MNNFQGLRSF¹⁴], SEQ ID NO:5 [M⁵NNFQGLRSF¹⁴], SEQ ID NO:6 [N⁶NFQGLRSF¹⁴], SEQ ID NO:7 [N7FQGLRSF¹⁴], SEQ ID NO:8 [F⁸QGLRSF¹⁴], SEQ ID NO:9 [Q⁹GLRSF¹⁴], SEQ ID NO:10 [G¹⁰LRSF¹⁴], SEQ ID NO:11 [L¹¹RSF¹⁴], SEQ ID NO:12 [R¹²SF¹⁴], and SEQ ID NO:13 [S¹³F¹⁴], which is covalently linked by an amide bond to the N-terminal G¹⁵ of the amino acid sequence of formula (I), wherein any amino acid of X² may optionally be replaced by a natural or unnatural amino acid;
wherein A is L-Alanine; R is L-Arginine; N is L-Asparagine; D is L-Aspartic acid; Q is L-Glutamine; G is L-Glycine; H is L-Histidine; I is L-Isoleucine; L is L-Leucine; K is L-Lysine; M is L-Methionine; F is L-Phenylalanine; P is L-Proline; S is L-Serine; T is L-Threonine; Y is L-Tyrosine; V is L-Valine;
wherein the numbering of amino acids in formula (I) and in the definition of X² refers to the corresponding human ADM sequence;
X³ is absent or is a heterologous moiety which is covalently linked to the N-terminus or to a functional group of the side chain of any amino acid of X², to the N-terminus of G¹⁵ or to Z;
Z is absent or is a cleavable linker covalently bound between the N terminus of any amino acid of X² or of G¹⁵ and X³ or between a functional group of the side chain of any amino acid of X² and X³
wherein if X³ is absent, then
Z is also absent and X² is hydrogen or is an amino acid or amino acid sequence as defined above;
   wherein if X³ is a heterologous moiety, then
X² is absent or is an amino acid or amino acid sequence as defined above; Z is absent or is a cleavable linker covalently bound between the N terminus of any amino acid of X² or of G¹⁵ and X³ or between a functional group of the side chain of any amino acid of X² and X³;
   or a physiologically acceptable salt, a solvate or a solvate of a salt thereof.

There are reports that the substitution of disulfide bonds with lactam bridges, as well as the introduction of N-methylation and palmitoylation may increase the metabolic stability of the peptides while retaining the biological activity. However, as reported e.g. by Watkins H A, Rathbone D L, Barwell J, Hay D L, Poyner D R, Structure-activity relationships for α-calcitonin gene-related peptide, Br J Pharmacol. 2013, 170(7), 1308-1322 and Dennis T, Fournier A, St Pierre S, Quirion R, Structure-activity profile of calcitonin gene-related peptide in peripheral and brain tissues. Evidence for receptor multiplicity. J. Pharmacol Exp Ther. 1989, 251(2), 718-725, the replacement of the disulfide bridge in members of the calcitonin superfamily of peptides was not correlated with retained activity. Also, while single changes of peptide structures are described, combinations of e.g. disulfide bond mimics, N-methylation and/or palmitoylation are not predictable with regard to structure-activity relationships.

ADM and other members of the calcitonin related peptides are known for fast inactivation by cleavage of the disulfide bridge. However, the activity retaining and at the same time half-life extending substitution of this disulfide bridge—even with alteration of the size of the intramolecular ring—is not known in the art and would not have been expected.

Compounds according to the invention are the compounds of the formula (I) and the salts thereof, solvates thereof and solvates of the salts thereof, the compounds which are embraced by formula (I) and are of the formulae specified below and the salts thereof, solvates thereof and solvates of the salts thereof, and the compounds which are embraced by formula (I) and are specified below as working examples and salts thereof, solvates thereof and solvates of the salts thereof, if the compounds which are embraced by formula (I) and are specified below are not already salts, solvates and solvates of the salts.

Depending on their structure, the compounds according to the invention may exist in stereoisomeric forms (enantiomers, diastereomers). The invention therefore embraces the enantiomers or diastereomers and the particular mixtures thereof. The stereoisomerically homogeneous constituents can be isolated in a known manner from such mixtures of enantiomers and/or diastereomers.

When the compounds according to the invention can occur in tautomeric forms, the present invention embraces all tautomeric forms.

Examples of stereoisomeric forms of the compounds of formula (I) according to the invention are compounds of the formulae (I) as defined above, wherein all amino acids have the L-configuration:

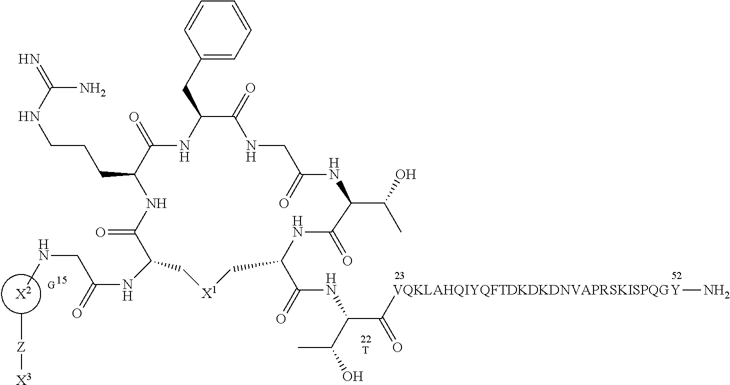

The present invention comprises all possible stereoisomeric forms, also in cases where no stereoisomerism is indicated.

The present invention also encompasses all suitable isotopic variants of the compounds of formula (I) according to the invention. An isotopic variant of a compound according to the invention is understood here to mean a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds of formula (I) according to the invention may therefore in some cases also constitute a preferred embodiment of the present invention. Isotopic variants of the compounds of formula (I) according to the invention can be prepared by processes known to those skilled in the art, for example by the methods described below and the methods described in the working examples, by using corresponding isotopic modifications of the particular reagents and/or starting compounds therein.

The present invention moreover also includes prodrugs of the compounds of formula (I) according to the invention. The term “prodrugs” here designates compounds which themselves can be biologically active or inactive, but are converted (for example metabolically or hydrolytically) into compounds of formula (I) according to the invention during their dwell time in the body. In the context of the present invention, preferred salts are physiologically acceptable salts of the compounds according to the invention. Also included are salts which are not suitable themselves for pharmaceutical applications, but, for example, can be used for the isolation or purification of the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluene-sulfonic acid, benzenesulfonic acid, naphthalenedisulfonic acid, acetic acid, trifluoroacetic acid, propionic acid, lactic acid, tartaric acid, maleic acid, citric acid, fumaric acid, maleic acid and benzoic acid.

Physiologically acceptable salts of the compounds according to the invention also include salts of customary bases, for example and with preference alkali metal salts (e.g. sodium and potassium salts), alkaline earth metal salts (e.g. calcium and magnesium salts) and ammonium salts derived from ammonia or organic amines having 1 to 16 carbon atoms, for example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates refer to those forms of the compounds according to the invention which, in the solid or liquid state, form a complex by coordination with solvent molecules. Hydrates are a specific form of the solvates, in which the coordination is with water. Preferred solvates in the context of the present invention are hydrates.

The specific radical definitions given in the particular combinations or preferred combinations of radicals are, irrespective of the particular combination of the radical specified, also replaced by any radical definitions of other combinations.

Very particular preference is given to combinations of two or more of the abovementioned preferred ranges.

The invention further provides a process for preparing the compounds of the formula (I) and (Ia), or salts thereof, solvates thereof or the solvates of salts thereof, wherein the compounds of the formula (II)

According to an embodiment of the invention, the compounds of formula (I) are defined as follows:

X¹ is selected from the group consisting of
*—(CH₂)ₘ₁—S—^{#}, wherein m1 is 0-6; ^{#}—(CH₂)ₘ₂—S—*, wherein m2 is 0-6;
*—(CH₂)ₘ₃—^{#}, wherein m3 is 1-8;
*—(CH₂)ₘ₄—(CH₂═CH₂)—(CH₂)ₙ₁—^{#}, wherein m4 is 0-6, n1 is 0-6, with the proviso that m4+n1=0-6;
*—(CH₂)ₘ₅—(CH≡CH)—(CH₂)ₙ₂—^{#}, wherein m5 is 0-6, and n2 is 0-6, with the proviso that m5+n2=0-6;
*—(CH₂)ₘ₆—CO—NH—(CH₂)ₙ₃—^{#}, wherein m6 is 0-4, and n3 is 0-4, with the proviso that m6+n3=0-6; ^{#}—(CH₂)ₘ₇—CO—NH—(CH₂)ₙ₄—*, wherein m7 is 0-4, and n4 is 0-4, with the proviso that m7+n4=0-6;
*—SO—(CH₂)ₘ₈—^{#}, wherein m8 is 0-6; ^{#}—SO—(CH₂)ₘ₉—*, wherein m9 is 0-6;
*—SO₂—(CH₂)ₘ₁₀—^{#}, wherein m10 is 0-6; ^{#}—SO₂—(CH₂)ₘ₁₁—*, wherein m11 is 0-6;

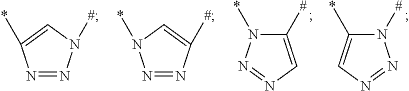

wherein * and ^{#} indicate the direction of binding within the ring, respectively, and
X², X³ and Z are as defined above;
or a physiologically acceptable salt, a solvate or a solvate of a salt thereof.

According to an embodiment of the invention, the compounds of formula (I) are defined as follows:

X¹ is selected from the group consisting of
*—(CH₂)ₘ₁—S—^{#}, wherein m1 is 0-4; ^{#}—(CH₂)ₘ₂—S—*, wherein m2 is 0-4;
*—(CH₂)ₘ₃—^{#}, wherein m3 is 1-6;
*—(CH₂)ₘ₄—(CH₂═CH₂)—(CH₂)ₙ₁—^{#}, wherein m4 is 0-4, n1 is 0-4, with the proviso that m4+n1=0-4;
*—(CH₂)ₘ₅—(CH≡CH)—(CH₂)ₙ₂—^{#}, wherein m5 is 0-4, and n2 is 0-4, with the proviso that m5+n2=0-4;
*—(CH₂)ₘ₆—CO—NH—(CH₂)ₙ₃—^{#}, wherein m6 is 0-4, and n3 is 0-4, with the proviso that m6+n3=0-4; ^{#}—(CH₂)ₘ₇—CO—NH—(CH₂)ₙ₄—*, wherein m7 is 0-4, and n4 is 0-4, with the proviso that m7+n4=0-4;
*—SO—(CH₂)ₘ₈—^{#}, wherein m8 is 0-4; ^{#}—SO—(CH₂)ₘ₉—*, wherein m9 is 0-4;
*—SO₂—(CH₂)ₘ₁₀—^{#}, wherein m10 is 0-4; ^{#}—SO₂—(CH₂)ₘ₁₁—*, wherein m11 is 0-4;

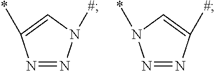

wherein * and ^{#} indicate the direction of binding within the ring, respectively, and
X², X³ and Z are as defined above;
or a physiologically acceptable salt, a solvate or a solvate of a salt thereof.

According to an embodiment of the invention, the compounds of formula (I) are defined as follows:

X¹ is selected from the group consisting of
*—(CH₂)ₘ₁—S—^{#}, wherein m1 is 0-6; ^{#}—(CH₂)ₘ₂—S—*, wherein m2 is 0-6;
*—(CH₂)ₘ₃—^{#}, wherein m3 is 1-8;
*—(CH₂)ₘ₆—CO—NH—(CH₂)ₙ₃—^{#}, wherein m6 is 0-4, and n3 is 0-4, with the proviso that m6+n3=0-6;
^{#}—(CH₂)ₘ₇—CO—NH—(CH₂)ₙ₄—*, wherein m7 is 0-4, and n4 is 0-4, with the proviso that m7+n4=0-6;
X² is G¹⁴ or K¹⁴, which is covalently linked by an amide bond to the N-terminal G¹⁵ of the compound of formula (I);
X³ is absent or is a heterologous moiety which is covalently linked to the N-terminus of G¹⁴ or K¹⁴ or to a functional group of the side chain of K¹⁴, or to Z;
Z is absent or is a cleavable linker covalently bound between the N terminus of G¹⁴ or K¹⁴ and X³, or between a functional group of the side chain of K¹⁴ and X³;
wherein if X³ is absent, then Z is also absent;
wherein if X³ is a heterologous moiety, then Z is absent or is a cleavable linker covalently bound between the N terminus of G¹⁴ or K¹⁴ and X³, or between a functional group of the side chain of K¹⁴ and X³;
or a physiologically acceptable salt, a solvate or a solvate of a salt thereof.

According to an embodiment of the invention, the compounds of formula (I) are defined as follows:

X¹ is selected from the group consisting of
*—CH₂—S—^{#}; ^{#}—CH₂—S—*; *—(CH₂)₂—^{#};
*—(CH₂)ₘ₆—CO—NH—(CH₂)ₙ₃—^{#}, wherein m6 is 0 or 1 and n3 is selected from 0, 1, 2, 3;
^{#}—(CH₂)ₘ₇—CO—NH—(CH₂)ₙ₄—*, wherein m7 is 0 or 1 and n4 is selected from 0, 1, 2, 3;
wherein * and ^{#} indicate the direction of binding within the ring, respectively, and X², X³ and Z are as defined above;
or a physiologically acceptable salt, a solvate or a solvate of a salt thereof.

According to an embodiment of the invention, the compounds of formula (I) are defined as follows:

X¹ is selected from the group consisting of
*—CH₂—S—^{#}; ^{#}—CH₂—S—*; *—(CH₂)₂—^{#};
*—(CH₂)ₘ₆—CO—NH—(CH₂)ₙ₃—^{#}, wherein m6 is 1 and n3 is selected from 0, 1, and 3; or m6 is 0 and n3 is selected from 0, 1, and 2;
^{#}—(CH₂)ₘ₇—CO—NH—(CH₂)ₙ₄—*, wherein m7 is 1 and n4 is selected from 0, 1, and 3; or m7 is 0 and n4 is selected from 0, 1, and 2;
wherein * and ^{#} indicate the direction of binding within the ring, respectively, and X², X³ and Z are as defined above;
or a physiologically acceptable salt, a solvate or a solvate of a salt thereof.

According to an embodiment of the invention, the compounds of formula (I) are defined as follows:

X¹ is selected from the group consisting of
*—CH₂—S—^{#}; ^{#}—CH₂—S—*;
*—CO—NH—CH₂—^{#}; *—CO—NH—(CH₂)₂—^{#}; *—CH₂—CO—NH—(CH₂)₃—^{#}; *—CH₂—CO—NH—CH₂—^{#};
^{#}—CH₂—CO—NH*;
wherein * and ^{#} indicate the direction of binding within the ring, respectively;
X² is as defined above;
X³ is absent or is palmitic acid; and
Z is absent;
or a physiologically acceptable salt, a solvate or a solvate of a salt thereof.

According to an embodiment of the invention, the compounds of formula (I) are defined as follows:

X¹ is selected from the group consisting of
*—(CH₂)ₘ₁—S—^{#}, wherein m1 is 0-6; ^{#}—(CH₂)ₘ₂—S—*, wherein m2 is 0-6;
*—(CH₂)ₘ₃—^{#}, wherein m3 is 1-8;
*—(CH₂)ₘ₆—CO—NH—(CH₂)ₙ₃—^{#}, wherein m6 is 0-4, and n3 is 0-4, with the proviso that m6+n3=0-6;
^{#}—(CH₂)ₘ₇—CO—NH—(CH₂)ₙ₄—*, wherein m7 is 0-4, and n4 is 0-4, with the proviso that m7+n4=0-6; and
X², X³ and Z are as defined above;
or a physiologically acceptable salt, a solvate or a solvate of a salt thereof.

According to an embodiment of the invention, the compounds of formula (I) are defined as follows:

X¹ is selected from the group consisting of
*—(CH₂)ₘ₁—S—^{#}, wherein m1 is 0-4; ^{#}—(CH₂)ₘ₂—S—*, wherein m2 is 0-4;
*—(CH₂)ₘ₆—CO—NH—(CH₂)ₙ₃—^{#}, wherein m6 is 0-4, and n3 is 0-4, with the proviso that m6+n3=0-6;
X² is G¹⁴ or K¹⁴, which is covalently linked by an amide bond to the N-terminal G¹⁵ of the compound of formula (I);
X³ is absent or is a heterologous moiety which is covalently linked to the N-terminus of G¹⁴ or K¹⁴ or to a functional group of the side chain of K¹⁴, or to Z;
Z is absent or is a cleavable linker covalently bound between the N terminus of G¹⁴ or K¹⁴ and X³, or between a functional group of the side chain of K¹⁴ and X³;
wherein if X³ is absent, then Z is also absent;
wherein if X³ is a heterologous moiety, then Z is absent or is a cleavable linker covalently bound between the N terminus of G¹⁴ or K¹⁴ and X³, or between a functional group of the side chain of K¹⁴ and X³;
or a physiologically acceptable salt, a solvate or a solvate of a salt thereof.

According to an embodiment of the invention, the compounds of formula (I) are defined as follows:

X¹ is selected from the group consisting of
*—(CH₂)—S—^{#}; ^{#}—(CH₂)—S—*;
*—(CH₂)ₘ₆—CO—NH—(CH₂)ₙ₃—^{#}, wherein m6 is 0 or 1, and n3 is selected from 1, 2, and 3;
X² is G¹⁴ or K¹⁴, which is covalently linked by an amide bond to the N-terminal G¹⁵ of the compound of formula (I);
X³ is absent or is a heterologous moiety which is covalently linked to the N-terminus of G¹⁴ or K¹⁴ or to a functional group of the side chain of K¹⁴, or to Z;
Z is absent or is a cleavable linker covalently bound between the N terminus of G¹⁴ or K¹⁴ and X³, or between a functional group of the side chain of K¹⁴ and X³;
wherein if X³ is absent, then Z is also absent;
wherein if X³ is a heterologous moiety, then Z is absent or is a cleavable linker covalently bound between the N terminus of G¹⁴ or K¹⁴ and X³, or between a functional group of the side chain of K¹⁴ and X³;
or a physiologically acceptable salt, a solvate or a solvate of a salt thereof.

According to an embodiment of the invention, the compounds of formula (I) are defined as follows:

X¹ is selected from the group consisting of
*—(CH₂)—S—^{#}; ^{#}—(CH₂)—S—*;
*—(CH₂)ₘ₆—CO—NH—(CH₂)ₙ₃—^{#}, wherein m6 is 0 or 1, and n3 is selected from 1, 2, and 3;
X² is G¹⁴ or K¹⁴, which is covalently linked by an amide bond to the N-terminal G¹⁵ of the compound of formula (I);
X³ is palmitic acid which is covalently linked to the N-terminus of G¹⁴ or K¹⁴ or to a functional group of the side chain of K¹⁴;
Z is absent;
or a physiologically acceptable salt, a solvate or a solvate of a salt thereof.

According to an embodiment of the invention, the compounds of formula (I) are defined as follows:

X¹ is as defined above;
X² is G¹⁴ or K¹⁴, which is covalently linked by an amide bond to the N-terminal G¹⁵ of the compound of formula (I); and
X³ and Z are as defined above;
or a physiologically acceptable salt, a solvate or a solvate of a salt thereof.

According to an embodiment of the invention, the compounds of formula (I) are defined as follows:

X¹ and X² are as defined above;
X³ is a polymer and the polymer is selected from the group consisting of linear or branched C₃-C₁₀₀ carboxylic acids, preferably C₄-C₃₀ carboxylic acids, optionally substituted with halo, hydroxy, alkoxy, amino, alkylamino, dialkylamino, sulfate, or phosphate, and which may be saturated, or mono- or di-unsaturated; and
Z is as defined above;
or a physiologically acceptable salt, a solvate or a solvate of a salt thereof.

According to an embodiment of the invention, the compounds of formula (I) are defined as follows:

X¹ and X² are as defined above;
X³ is a polymer and the polymer is a PEG moiety; and
Z is as defined above;
or a physiologically acceptable salt, a solvate or a solvate of a salt thereof.

According to an embodiment of the invention, the compounds of formula (I) are defined as follows:

X¹ and X² are as defined above;
X³ is a heterologous moiety; and
Z is a cleavable linker covalently bound between the N terminus of any amino acid of X² or of G¹⁵ and X³ or between a functional group of the side chain of any amino acid of X² and X³;
or a physiologically acceptable salt, a solvate or a solvate of a salt thereof.

According to an embodiment of the invention, the compounds of formula (I) are defined as follows:

at least one of the amino acids of X² has been replaced by a natural or by an unnatural amino acid; and
X¹, X³, and Z are as defined above;
or a physiologically acceptable salt, a solvate or a solvate of a salt thereof

Within the meaning of the present invention, natural amino acids are defined as peptidogenic amino acids. Within the meaning of the present invention, unnatural amino acids are defined as non-peptidogenic amino acids inserted in the peptides according to the invention, including:

Diaminodiacids, which are within the meaning of this invention defined as amino acids having two amino and two carboxyl groups. Diaminodiacids can form amide bonds with two further amino acids. Examples for diaminodiacids are cystathionine and 2,7-diaminosuberic acid;
Diaminoacids, which are within the meaning of this invention defined as amino acids having a second amino group. Examples for Diaminoacids are 3-aminoalanine (Dpr), 2,4-diaminobutyric acid (Dab), alpha, gamma diamino butyric acid (Dbu), and 2,5 Diaminopentanoic acid (Orn); D-amino acids, heterocyclic substituted alanine being used as replacement for phenylalanine, and halogenated amino acids.

According to an embodiment of the invention, the compound of formula (I) is defined as follows:

X³ is a heterologous moiety selected from the group consisting of a polymer, a Fc, a FcRn binding ligand, albumin and an albumin-binding ligand;
and X¹, X², and Z are as defined above;
or a physiologically acceptable salt, a solvate or a solvate of a salt thereof.

Within the meaning of the present invention, the term “heterologous moieties” includes a polymer, a Fc, a FcRn binding ligand, albumin and an albumin-binding ligand.

According to an embodiment of the invention, the compounds of formula (I) are defined as follows:

X³ is a polymer and the polymer is selected from the group consisting of linear or branched C₃-C₁₀₀ carboxylic acids, preferably C₄-C₃₀ carboxylic acids, optionally substituted with halo, hydroxy, alkoxy, amino, alkylamino, dialkylamino, sulfate, or phosphate, and which may be saturated, or mono- or di-unsaturated, a PEG moiety, a PPG moiety, a PAS moiety and a HES moiety; and
X¹, X², and Z are as defined above;
or a physiologically acceptable salt, a solvate or a solvate of a salt thereof.

According to an embodiment of the invention, the compounds of formula (I) are defined as follows:

X³ is a carboxylic acid selected from the group consisting of arachidic acid, arachidonic acid, behenic acid, capric acid, caproic acid, caprylic acid, ceroplastic acid, cerotic acid, docosahexaenoic acid, eicosapentaenoic acid, elaidic acid, enanthic acid, erucic acid, geddic acid, henatriacontylic acid, heneicosylic acid, heptacosylic acid, hexatriacontylic acid, lacceroic acid, lauric acid, lignoceric acid, linoelaidic acid, linoleic acid, margaric acid, melissic acid, montanic acid, myristic acid, myristoleic acid, nonacosylic acid, nonadecylic acid, oleic acid, palmitic acid, palmitoleic acid, pantothenic acid, pelargonic acid, pentacosylic acid, pentadecylic acid, psyllic acid, sapienic acid, stearic acid, tricosylic acid, tridecylic acid, undecylic acid, vaccenic acid, valeric acid, α-linolenic acid and derivatives thereof; and
X¹, X², and Z are as defined above;
or a physiologically acceptable salt, a solvate or a solvate of a salt thereof.

According to a further embodiment of the invention, the heterologous moiety is a polyethyleneglycol (PEG) or polypropyleneglycol (PPG) moiety known in the art. The polymer can be of any molecular weight, and can be branched or unbranched.

For polyethylene glycol, in one embodiment, the molecular weight is between about 1 kDa and about 100 kDa for ease in handling and manufacturing. Other sizes may be used, depending on the desired profile (e.g., the duration of sustained release desired, the effects, if any on biological activity, the ease in handling, the degree or lack of antigenicity and other known effects of the polyethylene glycol to a peptide or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa. In some embodiments, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al, Nucleosides Nucleotides 18:2745-2750 (1999); and Caliceti et al., Bioconjug. Chem. 10:638-646 (1999).

In other embodiments, the heterologous moiety is a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the procoagulant compound. Yet, the skilled person is aware that an amino acid polymer also may form random coil conformation when residues other than alanine, serine, and proline are added as a minor constituent in the PAS sequence. The term “minor constituent” as used herein means that amino acids other than alanine, serine, and proline may be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%>, i.e., about 9 of 100 amino acids, up to about 8%>, i.e., about 8 of 100 amino acids, about 6%>, i.e., about 6 of 100 amino acids, about 5%>, i.e., about 5 of 100 amino acids, about 4%>, i.e., about 4 of 100 amino acids, about 3%>, i.e., about 3 of 100 amino acids, about 2%>, i.e., about 2 of 100 amino acids, about 1%>, i.e., about 1 of 100 of the amino acids. The amino acids different from alanine, serine and proline may be selected from the group consisting of Arg, Asn, Asp, Cys, Gin, Glu, Gly, His, He, Leu, Lys, Met, Phe, Thr, Trp, Tyr, and Val. Under physiological conditions, the PAS sequence stretch forms a random coil conformation and thereby can mediate an increased in vivo and/or in vitro stability to procoagulant compound. Since the random coil domain does not adopt a stable structure or function by itself, the biological activity mediated by the Pep1 and/or Pep2 polypeptides in the procoagulant compound is essentially preserved. In other embodiments, the PAS sequences that form random coil domain are biologically inert, especially with respect to proteolysis in blood plasma, immunogenicity, isoelectric point/electrostatic behaviour, binding to cell surface receptors or internalisation, but are still biodegradable, which provides clear advantages over synthetic polymers such as PEG.

Non-limiting examples of the PAS sequences forming random coil conformation comprise an amino acid sequence selected from the group consisting of ASPAAPAPASPAAPAPSAPA, AAPASPAPAAPSAPAPAAPS, APSSPSPSAPSSPSPASPSS, APSSPSPSAPSSPSPASPS, SSPSAPSPSSPASPSPSSPA, AASPAAPSAPPAAASPAAPSAPPA, and AS AAAP AAAS AAAS AP S AAA, or any combinations thereof. Additional examples of PAS sequences are known from, e.g., US Pat. Publ. No. 2010/0292130 A1 and PCT Appl. Publ. No. WO 2008/155134 A1.

In certain embodiments, the heterologous moiety is hydroxyethyl starch (HES) or a derivative thereof. Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., Krankenhauspharmazie, 8(8), 271-278 (1987); and Weidler et al, Arzneim.-Forschung/Drug Res., 41, 494-498 (1991)).

Amylopectin contains glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. The degree of substitution, denoted as DS, relates to the molar substitution, is known to the skilled people. See Sommermeyer et ah, Krankenhauspharmazie, 8(8), 271-278 (1987), as cited above, in particular p. 273.

In one embodiment, hydroxyethyl starch has a mean molecular weight (weight mean) of from 1 to 300 kD, from 2 to 200 kD, from 3 to 100 kD, or from 4 to 70 kD. hydroxyethyl starch can further exhibit a molar degree of substitution of from 0.1 to 3, preferably 0.1 to 2, more preferred, 0.1 to 0.9, preferably 0.1 to 0.8, and a ratio between C2:C6 substitution in the range of from 2 to 20 with respect to the hydroxyethyl groups. A non-limiting example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7. In a specific embodiment, HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolemia. The characteristics of VOLUVEN® are a mean molecular weight of 130,000+/−20,000 D, a molar substitution of 0.4 and a C2:C6 ratio of about 9:1. In other embodiments, ranges of the mean molecular weight of hydroxyethyl starch are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD. In still other embodiments, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD) or from 12 to 13 kD or from 11 to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD.

In certain embodiments, the heterologous moiety can be a mixture of hydroxyethyl starches having different mean molecular weights and/or different degrees of substitution and/or different ratios of C2:C6 substitution. Therefore, mixtures of hydroxyethyl starches may be employed having different mean molecular weights and different degrees of substitution and different ratios of C2:C6 substitution, or having different mean molecular weights and different degrees of substitution and the same or about the same ratio of C2:C6 substitution, or having different mean molecular weights and the same or about the same degree of substitution and different ratios of C2:C6 substitution, or having the same or about the same mean molecular weight and different degrees of substitution and different ratios of C2:C6 substitution, or having different mean molecular weights and the same or about the same degree of substitution and the same or about the same ratio of C2:C6 substitution, or having the same or about the same mean molecular weights and different degrees of substitution and the same or about the same ratio of C2:C6 substitution, or having the same or about the same mean molecular weight and the same or about the same degree of substitution and different ratios of C2:C6 substitution, or having about the same mean molecular weight and about the same degree of substitution and about the same ratio of C2:C6 substitution.

In certain embodiments, the heterologous moiety is a polysialic acids (PSAs) or a derivative thereof. Polysialic acids (PSAs) are naturally occurring unbranched polymers of sialic acid produced by certain bacterial strains and in mammals in certain cells Roth J., et al. (1993) in Polysialic Acid: From Microbes to Man, eds Roth J., Rutishauser U., Troy F. A. (Birkhauser Verlag, Basel, Switzerland), pp 335-348. They can be produced in various degrees of polymerisation from n=about 80 or more sialic acid residues down to n=2 by limited acid hydrolysis or by digestion with neuraminidases, or by fractionation of the natural, bacterially derived forms of the polymer. The composition of different polysialic acids also varies such that there are homopolymeric forms i.e. the alpha-2,8-linked polysialic acid comprising the capsular polysaccharide of E. coli strain K1 and the group-B meningococci, which is also found on the embryonic form of the neuronal cell adhesion molecule (N-CAM). Heteropolymeric forms also exist—such as the alternating alpha-2,8 alpha-2,9 polysialic acid of E. coli strain K92 and group C polysaccharides of N. meningitidis. Sialic acid may also be found in alternating copolymers with monomers other than sialic acid such as group W 135 or group Y of N. meningitidis. Polysialic acids have important biological functions including the evasion of the immune and complement systems by pathogenic bacteria and the regulation of glial adhesiveness of immature neurons during foetal development (wherein the polymer has an anti-adhesive function) Cho and Troy, P.N.A.S., USA, 91 (1994) 11427-11431, although there are no known receptors for polysialic acids in mammals. The alpha-2,8-linked polysialic acid of E. coli strain K1 is also known as ‘colominic acid’ and is used (in various lengths) to exemplify the present invention. Various methods of attaching or conjugating polysialic acids to a peptide or polypeptide have been described (for example, see U.S. Pat. No. 5,846,951; WO-A-0187922, and US 2007/0191597 A1.

In certain embodiments, the heterologous moiety is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)n, (Gly4Ser)n or S(Gly4Ser)n, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200.

In certain aspects, a compound of the invention is covalently linked to at least one heterologous moiety that is or comprises an XTEN polypeptide or fragment, variant, or derivative thereof. As used here “XTEN polypeptide” refers to extended length polypeptides with non-naturally occurring, substantially non-repetitive sequences that are composed mainly of small hydrophilic amino acids, with the sequence having a low degree or no secondary or tertiary structure under physiologic conditions. As a heterologous moiety, XTENs can serve as a half-life extension moiety. In addition, XTEN can provide desirable properties including but are not limited to enhanced pharmacokinetic parameters and solubility characteristics.

The incorporation of a heterologous moiety comprising an XTEN sequence into a conjugate of the invention can confer one or more of the following advantageous properties to the resulting conjugate: conformational flexibility, enhanced aqueous solubility, high degree of protease resistance, low immunogenicity, low binding to mammalian receptors, or increased hydrodynamic (or Stokes) radii.

In certain aspects, an XTEN moiety can increase pharmacokinetic properties such as longer in vivo half-life or increased area under the curve (AUC), so that a compound or conjugate of the invention stays in vivo and has procoagulant activity for an increased period of time compared to a compound or conjugate with the same but without the XTEN heterologous moiety.

Examples of XTEN moieties that can be used as heterologous moieties in procoagulant conjugates of the invention are disclosed, e.g., in U.S. Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, or WO 2011028344 A2.

Within the meaning of the present invention, the term “Fc” is to be understood as immunoglobulin constant region or a portion thereof, such as an Fc region or a FcRn binding partner. In certain embodiments, the compound or conjugate is linked to one or more truncated Fc regions that are nonetheless sufficient to confer Fc receptor (FcR) binding properties to the Fc region. For example, the portion of an Fc region that binds to FcRn (i.e., the FcRn binding portion) comprises from about amino acids 282-438 of IgG1, EU numbering (with the primary contact sites being amino acids 248, 250-257, 272, 285, 288, 290-291, 308-311, and 314 of the CH2 domain and amino acid residues 385-387, 428, and 433-436 of the CH3 domain. Thus, an Fc region in a biologically active ADM peptide derivative of the invention may comprise or consist of an FcRn binding portion. FcRn binding portions may be derived from heavy chains of any isotype, including IgG1, IgG2, IgG3 and IgG4. In one embodiment, an FcRn binding portion from an antibody of the human isotype IgG1 is used. In another embodiment, an FcRn binding portion from an antibody of the human isotype IgG4 is used.

In certain embodiments, an Fc region comprises at least one of: a hinge (e.g., upper, middle, and/or lower hinge region) domain (about amino acids 216-230 of an antibody Fc region according to EU numbering), a CH2 domain (about amino acids 231-340 of an antibody Fc region according to EU numbering), a CH3 domain (about amino acids 341-438 of an antibody Fc region according to EU numbering), a CH4 domain, or a variant, portion, or fragment thereof. In other embodiments, an Fc region comprises a complete Fc domain (i.e., a hinge domain, a CH2 domain, and a CH3 domain). In some embodiments, an Fc region comprises, consists essentially of, or consists of a hinge domain (or a portion thereof) fused to a CH3 domain (or a portion thereof), a hinge domain (or a portion thereof) fused to a CH2 domain (or a portion thereof), a CH2 domain (or a portion thereof) fused to a CH3 domain (or a portion thereof), a CH2 domain (or a portion thereof) fused to both a hinge domain (or a portion thereof) and a CH3 domain (or a portion thereof). In still other embodiments, an Fc region lacks at least a portion of a CH2 domain (e.g., all or part of a CH2 domain). In a particular embodiment, an Fc region comprises or consists of amino acids corresponding to EU numbers 221 to 447.

An Fc in a biologically active ADM peptide derivative of the invention can include, for example, a change (e.g., a substitution) at one or more of the amino acid positions disclosed in Int'l. PCT Publications WO88/07089A1, WO96/14339A1, WO98/05787A1, WO98/23289A1, WO99/51642A1, WO99/58572A1, WO00/09560A2, WO0/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; U.S. Pat. Publ. Nos. US 2007/0231329, US2007/0231329, US2007/0237765, US2007/0237766, US2007/0237767, US2007/0243188, US2007/0248603, US2007/0286859, US2008/0057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091. In one embodiment, the specific change (e.g., the specific substitution of one or more amino acids disclosed in the art) may be made at one or more of the disclosed amino acid positions. In another embodiment, a different change at one or more of the disclosed amino acid positions (e.g., the different substitution of one or more amino acid position disclosed in the art) may be made.

An Fc region used in the invention may also comprise an art recognized amino acid substitution which alters its glycosylation. For example, the Fc has a mutation leading to reduced glycosylation (e.g., N- or O-linked glycosylation) or may comprise an altered glycoform of the wild-type Fc moiety (e.g., a low fucose or fucose-free glycan).

In certain embodiments, the compound or conjugate of the invention is linked to a heterologous moiety comprising albumin or a functional fragment thereof. Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ. Nos. 2008/0194481A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2.

In one embodiment, the heterologous moiety is albumin, a fragment, or a variant thereof which is further linked to a heterologous moiety selected from the group consisting of an immunoglobulin constant region or portion thereof (e.g., an Fc region), a PAS sequence, HES, and PEG.

In certain embodiments, the heterologous moiety is an albumin binding moiety, which comprises an albumin binding peptide, a bacterial albumin binding domain, an albumin-binding antibody fragment, or any combinations thereof.

For example, the albumin binding protein can be a bacterial albumin binding protein, an antibody or an antibody fragment including domain antibodies (see U.S. Pat. No. 6,696,245). An albumin binding protein, for example, can be a bacterial albumin binding domain, such as the one of streptococcal protein G (Konig, T. and Skerra, A. (1998) J. Immunol. Methods 218, 73-83). Other examples of albumin binding peptides that can be used as conjugation partner are, for instance, those having a Cys-Xaa i-Xaa 2-Xaa 3-Xaa 4-Cys consensus sequence, wherein Xaa i is Asp, Asn, Ser, Thr, or Trp; Xaa 2 is Asn, Gin, H is, He, Leu, or Lys; Xaa 3 is Ala, Asp, Phe, Trp, or Tyr; and Xaa 4 is Asp, Gly, Leu, Phe, Ser, or Thr as described in US patent application 2003/0069395 or Dennis et al. (Dennis et al. (2002) J. Biol. Chem. 277, 35035-35043). Domain 3 from streptococcal protein G, as disclosed by Kraulis et al, FEBS Lett. 378: 190-194 (1996) and Linhult et al, Protein Sci. 11:206-213 (2002) is an example of a bacterial albumin-binding domain. Examples of albumin-binding peptides include a series of peptides having the core sequence DICLPRWGCLW (SEQ ID NO:45). See, e.g., Dennis et al, J. Biol. Chem. 2002, 277: 35035-35043 (2002). Examples of albumin-binding antibody fragments are disclosed in Muller and Kontermann, Curr. Opin. Mol. Ther. 9:319-326 (2007); Roovers et al, Cancer Immunol. Immunother. 56:303-317 (2007), and Holt et al, Prot. Eng. Design Sci., 21:283-288 (2008), which are incorporated herein by reference in their entireties. An example of such albumin binding moiety is 2-(3-maleimidopropanamido)-6-(4-(4-iodophenyl)butanamido) hexanoate (“Albu” tag) as disclosed by Trussel et al, Bioconjugate Chem. 20:2286-2292 (2009).

According to an embodiment of the present invention, the compounds of formula (I) are as defined as follows:

Z is absent and X¹, X², and X³ are as defined above;
or a physiologically acceptable salt, a solvate or a solvate of a salt thereof.

According to this embodiment, the heterologous moiety X³ as defined above is covalently linked to X² in a permanent manner. Within the meaning of the present invention, the term that a moiety is “covalently linked to the peptide in a permanent manner” is to be understood, that the moiety is covalently linked to the peptide without using a linker Z. An example is the functionalization of the N terminus or suitable side chain functionalities of any amino acid in the sequence of the peptide of formula (I) with a linear or branched C₃-C₁₀₀ carboxylic acid, preferably a C₄-C₃₀ carboxylic acid, optionally substituted with halo, hydroxy, alkoxy, amino, alkylamino, dialkylamino, sulfate, or phosphate, and which may be saturated, or mono- or di-unsaturated.

According to an embodiment of the present invention, the compounds of formula (I) are defined as follows:

Z is a cleavable linker as defined above; and X¹, X², and X³ are as defined above;
or a physiologically acceptable salt, a solvate or a solvate of a salt thereof.

Within the meaning of the present invention, the term “cleavable linker” is to be understood as a linker between X² and X³, which allows the heterologous moiety to be released from X² by an enzymatic process or by a pH-dependent nucleophilic process or by hydrolysis or by any combination thereof.

According to an embodiment of the present invention, the compounds of formula (I) are further modified by N-methylation of at least one amide bond.

The influence of N-methylation on the metabolic stability of peptides has been described for various peptides. For example, cyclosporine is a naturally occurring, cyclic, multiply N-methylated peptide that exhibits an excellent pharmacokinetic profile. N-methylation in general blocks enzymatic degradation by proteases as they are unable to cleave N-methylated peptide bonds. Multiple N-methylation was shown to improve the metabolic stability and intestinal permeability of peptides [Chatterjee J, Gilon C, Hoffman A, Kessler H, N-methylation of peptides: a new perspective in medicinal chemistry, Acc Chem Res., 41(10), 1331-1342, 2008]. Cyclization combined with N-methylation was used to modulate physicochemical properties of peptides, including metabolic stability, membrane permeability and oral bioavailability [Chatterjee J, Laufer B, Kessler H, Synthesis of N-methylated cyclic peptides, Nat Protoc., 7(3), 432-444, 2012]. Dong Q G, Zhang Y, Wang M S, Feng J, Zhang H H, Wu Y G, Gu T J, Yu X H, Jiang C L, Chen Y, Li W, Kong W, Improvement of enzymatic stability and intestinal permeability of deuterohemin-peptide conjugates by specific multi-site N-methylation, Amino Acids., 43(6), 2431-2441, 2012, describe that N-Methylation at selected sites showed high resistance against proteolytic degradation. In diluted serum and intestinal preparation 50- to 140-fold higher half-life values were observed. However, Linde Y, Ovadia O, Safrai E, Xiang Z, Portillo F P, Shalev D E, Haskell-Luevano C, Hoffman A, Gilon C, Structure-activity relationship and metabolic stability studies of backbone cyclization and N-methylation of melanocortin peptides, Biopolymers., 90(5), 671-682, 2008, describe that cyclic N-methylated analogues of the α-melanocyte stimulating hormone were more stable, however less biologically active than the parent peptide.

The compounds according to the invention show an unforeseeable useful spectrum of pharmacological activity.

Accordingly they are suitable for use as medicaments for treatment and/or prevention of diseases in humans and animals.

The present invention further provides for the use of the compounds according to the invention for treatment and/or prevention of disorders, especially of cardiovascular, edematous and/or inflammatory disorders.

For the present invention, the term “treatment” or “treating” includes inhibiting, delaying, relieving, mitigating, arresting, reducing, or causing the regression of a disease, disorder, condition, or state, the development and/or progression thereof, and/or the symptoms thereof. The term “prevention” or “preventing” includes reducing the risk of having, contracting, or experiencing, a disease, disorder, condition, or state, the development and/or progression thereof, and/or the symptoms thereof. The term prevention includes prophylaxis. Treatment or prevention of a disease, disorder, condition, or state may be partial or complete.

On the basis of their pharmacological properties, the compounds according to the invention can be employed for treatment and/or prevention of cardiovascular diseases, in particular heart failure, especially chronic and acute heart failure, worsening heart failure, diastolic and systolic (congestive) heart failure, acute decompensated heart failure, cardiac insufficiency, coronary heart disease, angina pectoris, myocardial infarction, ischemia reperfusion injury, ischemic and hemorrhagic stroke, arteriosclerosis, atherosclerosis, hypertension, especially essential hypertension, malignant essential hypertension, secondary hypertension, renovascular hypertension and hypertension secondary to renal and endocrine disorders, hypertensive heart disease, hypertensive renal disease, pulmonary hypertension, especially secondary pulmonary hypertension, pulmonary hypertension following pulmonary embolism with and without acute cor pulmonale, primary pulmonary hypertension, and peripheral arterial occlusive disease.

The compounds according to the invention are furthermore suitable for treatment and/or prevention of gestational [pregnancy-induced] edema and proteinuria with and without hypertension (pre-eclampsia).

The compounds according to the invention are furthermore suitable for treatment and/or prevention of pulmonary disorders, such as chronic obstructive pulmonary disease, asthma, acute and chronic pulmonary edema, allergic alveolitis and pneumonitis due to inhaled organic dust and particles of fungal, actinomycetic or other origin, acute chemical bronchitis, acute and chronic chemical pulmonary edema (e.g. after inhalation of phosgene, nitrogen oxide), neurogenic pulmonary edema, acute and chronic pulmonary manifestations due to radiation, acute and chronic interstitial lung disorders (such as but not restricted to drug-induced interstitial lung disorders, e.g. secondary to Bleomycin treatment), acute lung injury/acute respiratory distress syndrome (ALI/ARDS) in adult or child including newborn, ALI/ARDS secondary to pneumonia and sepsis, aspiration pneumonia and ALI/ARDS secondary to aspiration (such as but not restricted to aspiration pneumonia due to regurgitated gastric content), ALI/ARDS secondary to smoke gas inhalation, transfusion-related acute lung injury (TRALI), ALI/ARDS or acute pulmonary insufficiency following surgery, trauma or burns, ventilator induced lung injury (VILI), lung injury following meconium aspiration, pulmonary fibrosis, and mountain sickness.

The compounds according to the invention are furthermore suitable for treatment and/or prevention of chronic kidney diseases (stages 1-5), renal insufficiency, diabetic nephropathy, hypertensive chronic kidney disease, glomerulonephritis, rapidly progressive and chronic nephritic syndrome, unspecific nephritic syndrome, nephrotic syndrome, hereditary nephropathies, acute and chronic tubulo-interstitial nephritis, acute kidney injury, acute kidney failure, posttraumatic kidney failure, traumatic and postprocedural kidney injury, cardiorenal syndrome, and protection and functional improvement of kidney transplants.

The compounds are moreover suitable for treatment and/or prevention of diabetes mellitus and its consecutive symptoms, such as e.g. diabetic macro- and microangiopathy, diabetic nephropathy and neuropathy.

The compounds according to the invention can moreover be used for treatment and/or prevention of disorders of the central and peripheral nervous system such as viral and bacterial meningitis and encephalitis (e.g. Zoster encephalitis), traumatic and toxic brain injury, primary or secondary [metastasis] malignant neoplasm of the brain and spinal cord, radiculitis and polyradiculitis, Guillain-Barre syndrome [acute (post-)infective polyneuritis, Miller Fisher Syndrome], amyotrophic lateral sclerosis [progressive spinal muscle atrophy], Parkinson's disease, acute and chronic polyneuropathies, pain, cerebral edema, Alzheimer's disease, degenerative diseases of the nervous system and demyelinating diseases of the central nervous system such as but not restricted to multiple sclerosis.

The compounds according to the invention are furthermore suitable for treatment and/or prevention of portal hypertension and liver fibrosis [cirrhosis] and its sequelae such as esophageal varices and ascites, for the treatment and/or prevention of pleural effusions secondary to malignancies or inflammations and for the treatment and/or prevention of lymphedema and of edema secondary to varices.

The compounds according to the invention are furthermore suitable for treatment and/or prevention of inflammatory disorders of the gastrointestinal tract such as inflammatory bowel disease, Crohn's disease, ulcerative colitis, and toxic and vascular disorders of the intestine.

The compounds according to the invention are furthermore suitable for treatment and/or prevention of sepsis, septic shock, systemic inflammatory response syndrome (SIRS) of non-infectious origin, hemorrhagic shock, sepsis or SIRS with organ dysfunction or multi organ failure (MOF), traumatic shock, toxic shock, anaphylactic shock, urticaria, insect sting and bite-related allergies, angioneurotic edema [Giant urticaria, Quincke's edema], acute laryngitis and tracheitis, and acute obstructive laryngitis [croup] and epiglottitis.

The compounds are furthermore suitable for treatment and/or prevention of diseases of the rheumatic type and other disease forms to be counted as autoimmune diseases such as but not restricted to polyarthritis, lupus erythematodes, scleroderma, purpura and vasculitis.

The compounds according to the invention are furthermore suitable for treatment of edematous ocular disorders or ocular disorders associated with disturbed vascular function, including, but not being limited to, age-related macular degeneration (AMD), diabetic retinopathy, in particular diabetic macula edema (DME), subretinal edema, and intraretinal edema. In the context of the present invention, the term age-related macular degeneration (AMD) encompasses both wet (or exudative, neovascular) and dry (or non-exudative, non-neovascular) manifestations of AMD.

The compounds according to the invention are furthermore suitable for treatment of ocular hypertension (glaucoma).

The compounds according to the invention can moreover be used for treatment and/or prevention of operation-related states of ischemia and consecutive symptoms thereof after surgical interventions, in particular interventions on the heart using a heart-lung machine (e.g. bypass operations, heart valve implants), interventions on the carotid arteries, interventions on the aorta and interventions with instrumental opening or penetration of the skull cap.

The compounds are furthermore suitable for general treatment and/or prevention in the event of surgical interventions with the aim of accelerating wound healing and shortening the reconvalescence time. They are further suited for the promotion of wound healing.

The compounds are furthermore suitable for treatment and/or prevention of disorders of bone density and structure such as but not restricted to osteoporosis, osteomalacia and hyperparathyroidism-related bone disorders.

The compounds are furthermore suitable for treatment and/or prevention of sexual dysfunctions, in particular male erectile dysfunction.

Preferable the compounds are suitable for treatment and/or prevention of heart failure, chronic heart failure, worsening heart failure, acute heart failure, acute decompensated heart failure, diastolic and systolic (congestive) heart failure, coronary heart disease, ischemic and/or hemorrhagic stroke, hypertension, pulmonary hypertension, peripheral arterial occlusive disease, pre-eclampsia, chronic obstructive pulmonary disease, asthma, acute and/or chronic pulmonary edema, allergic alveolitis and/or pneumonitis due to inhaled organic dust and particles of fungal, actinomycetic or other origin, and/or acute chemical bronchitis, acute and/or chronic chemical pulmonary edema, neurogenic pulmonary edema, acute and/or chronic pulmonary manifestations due to radiation, acute and/or chronic interstitial lung disorders, acute lung injury/acute respiratory distress syndrome (ALI/ARDS) in adult or child including newborn, ALI/ARDS secondary to pneumonia and sepsis, aspiration pneumonia and ALI/ARDS secondary to aspiration, ALI/ARDS secondary to smoke gas inhalation, transfusion-related acute lung injury (TRALI), ALI/ARDS and/or acute pulmonary insufficiency following surgery, trauma and/or burns, and/or ventilator induced lung injury (VILI), lung injury following meconium aspiration, pulmonary fibrosis, mountain sickness, chronic kidney diseases, glomerulonephritis, acute kidney injury, cardiorenal syndrome, lymphedema, inflammatory bowel disease, sepsis, septic shock, systemic inflammatory response syndrome (SIRS) of non-infectious origin, anaphylactic shock, inflammatory bowel disease and/or urticaria.

More preferable the compounds are suitable for treatment and/or prevention of heart failure, chronic heart failure, worsening heart failure, acute heart failure, acute decompensated heart failure, diastolic and systolic (congestive) heart failure, hypertension, pulmonary hypertension, asthma, acute and/or chronic chemical pulmonary edema, acute lung injury/acute respiratory distress syndrome (ALI/ARDS) in adult or child including newborn, ALI/ARDS secondary to pneumonia and sepsis, aspiration pneumonia and ALI/ARDS secondary to aspiration, ALI/ARDS secondary to smoke gas inhalation, transfusion-related acute lung injury (TRALI), ALI/ARDS and/or acute pulmonary insufficiency following surgery, trauma and/or burns, and/or ventilator induced lung injury (VILI), lung injury following meconium aspiration, sepsis, septic shock, systemic inflammatory response syndrome (SIRS) of non-infectious origin, anaphylactic shock, inflammatory bowel disease and/or urticaria.

The present invention further provides for the use of the compounds according to the invention for treatment and/or prevention of disorders, in particular the disorders mentioned above.

The present invention further provides for the use of the compounds according to the invention for preparing a medicament for treatment and/or prevention of disorders, in particular the disorders mentioned above.

The present invention further provides a method for treatment and/or prevention of disorders, in particular the disorders mentioned above, using an active amount of the compounds according to the invention.

The invention further provides medicaments comprising a compound according to the invention and one or more further active ingredients, in particular for treatment and/or prevention of the disorders mentioned above. Exemplary and preferred active ingredient combinations are: ACE inhibitors, angiotensin receptor antagonists, beta-2 receptor agonists, phosphodiesterase inhibitors, glucocorticoid receptor agonists, diuretics, or recombinant angiotensin converting enzyme-2 or acetylsalicylic acid (aspirin).

In a preferred embodiment of the invention, the compounds according to the invention are administered in combination with an ACE inhibitor, such as, by way of example and preferably, enalapril, quinapril, captopril, lisinopril, ramipril, delapril, fosinopril, perindopril, cilazapril, imidapril, benazepril, moexipril, spirapril or trandopril.

In a preferred embodiment of the invention, the compounds according to the invention are administered in combination with an angiotensin receptor antagonist, such as, by way of example and preferably, losartan, candesartan, valsartan, telmisartan or embusartan.

In a preferred embodiment of the invention, the compounds according to the invention are administered in combination with a beta-2 receptor agonist, such as, by way of example and preferably, salbutamol, pirbuterol, salmeterol, terbutalin, fenoterol, tulobuterol, clenbuterol, reproterol or formoterol.

In a preferred embodiment of the invention, the compounds according to the invention are administered in combination with a phosphodiesterase (PDE) inhibitor, such as, by way of example and preferably, milrinone, amrinone, pimobendan, cilostazol, sildenafil, vardenafil or tadalafil.

In a preferred embodiment of the invention, the compounds according to the invention are administered in combination with a glucocorticoid receptor agonist, such as, by way of example and preferably, cortiosol, cortisone, hydrocortisone, prednisone, methyl-prednisolone, prednylidene, deflazacort, fluocortolone, triamcinolone, dexamethasone or betamethasone.

In a preferred embodiment of the invention, the compounds according to the invention are administered in combination with diuretics, such as, by way of example and preferably, furosemide, torasemide and hydrochlorothiazide.

In a preferred embodiment of the invention, the compounds according to the invention are administered in combination with natriuretic peptides, such as nesiritide (human B-type natriuretic peptide (hBNP)) and carperitide (alpha-human atrial natriuretic polypeptide (hANP)).

In a preferred embodiment of the invention, the compounds according to the invention are administered in combination with urodilatin, a derivative of ANP still under development for acute heart failure.

In a preferred embodiment of the invention, the compounds according to the invention are administered in combination with LCZ696 (Entresto), a neprilysin (enkephalinase, neutral endopeptidase, NEP, also involved in the metabolism of ADM) inhibitor.

The present invention further relates to medicaments which comprise at least one compound according to the invention, normally together with one or more inert, nontoxic, pharmaceutically suitable excipients and to the use thereof for the aforementioned purposes.

The compounds according to the invention can act systemically and/or locally. For this purpose, they can be administered in a suitable way, for example by the parenteral, pulmonary, nasal, sublingual, lingual, buccal, dermal, transdermal, conjunctival, optic route or as implant or stent.

The compounds according to the invention can be administered in administration forms suitable for these administration routes.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration include preparations for injection and infusion in the form of solutions, suspensions, emulsions, lyophilizates or sterile powders.

Suitable for the other administration routes are, for example, pharmaceutical forms for inhalation (including powder inhalers, nebulizers), nasal drops, eye drops, solutions or sprays; films/wafers or aqueous suspensions (lotions, shaking mixtures), lipophilic suspensions, ointments, creams, transdermal therapeutic systems (e.g. patches), milk, pastes, foams, dusting powders, implants or stents.

Parenteral administration is preferred, especially intravenous administration. Inhalative administration is also preferred, e.g. by using powder inhalers or nebulizers.

The compounds according to the invention can be converted into the stated administration forms. This can take place in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colors (e.g. inorganic pigments, for example iron oxides) and masking flavors and/or odors.

It has generally been found to be advantageous, in the case of parenteral administration, to administer amounts of about 0.001 to 5 mg/kg, preferably about 0.01 to 1 mg/kg, of body weight to achieve effective results.

It may nevertheless be necessary in some cases to deviate from the stated amounts; in particular as a function of the body weight, route of administration, individual response to the active ingredient, nature of the preparation and time or interval over which administration takes place. For instance, less than the aforementioned minimum amount may be sufficient in some cases, whereas in other cases the stated upper limit must be exceeded. In the case of administration of larger amounts, it may be advisable to divide these into a plurality of individual doses over the day.

The following working examples illustrate the invention. The invention is not restricted to the examples.

The percentages in the following tests and examples are, unless stated otherwise, percentages by weight; parts are parts by weight. Solvent ratios, dilution ratios and concentration data for the liquid/liquid solutions are each based on volume.

### EXPLANATION OF THE FIGURES

FIG. 1: Transcellular electrical resistance assays in endothelial cells (1b). Treatment with Example 16 reduced break down of electrical resistance of a HUVEC monolayer after stimulation with thrombin dose dependently and significantly at concentrations of ≧1 nmol/L. Values were plotted as means±SEM of 4 data points.

FIG. 2: In vitro-permeability assays in endothelial cells (1c). Treatment with Example 18 reduced permeability of a HUVEC monolayer for FITC-Dextran after stimulation with thrombin dose dependently and significantly at concentrations of ≧0.3 nmol/L. Values were plotted as means±SEM of at least 4 data points.

FIG. 3: Stability of peptides in plasma calculated with GraphPad Prism 5 (GraphPad Software) using two phase decay analysis for the determination of half-life (slow) (Test 1e). N-terminally 6-carboxytetramethylrhodamine-(TAM)-labeled analogues of Control: TAM[G¹⁴]ADM(14-52); Example 18: TAM[K¹⁴(PAM), (Dpr¹⁶, E²¹)lac]ADM(14-52); and Example 27: TAM[K¹⁴(PAM), (Dpr¹⁶, E²¹)lac, N_{α}-Me-K⁴⁶]ADM(14-52).

FIG. 4: Granulocyte transmigration assay (Test 1f). Treatment with Example 18 reduced transmigration of PMNS through TNF-α stimulated HUVECs significantly at concentrations 1≧nmol/L. Values were plotted as means±SEM of 7 replicas, vehicle control n=12. Anti ICAM-1 antibody served as positive control, n=6.

FIG. 5: Measurement of blood pressure and heart rate in telemetered, normotensive Wistar rats. (Test 2a) 24 hour profiles of mean arterial blood pressure (MABP) recorded from telemetered, normotensive female Wistar rats after subcutaneous administration of example 18 or vehicle at doses as indicated (dotted line). Data points were plotted as means±SEM of averaged 30 min intervals from 6 animals per group. Administration of Example 18 at a dose of 100 μg/kg reduced MABP by about 20 to 25% until 3.5 h after administration (filled circles). Between 4 h and 8 h after administration, MABP gradually returned to baseline values and finally was in the range of that of vehicle treated animals.

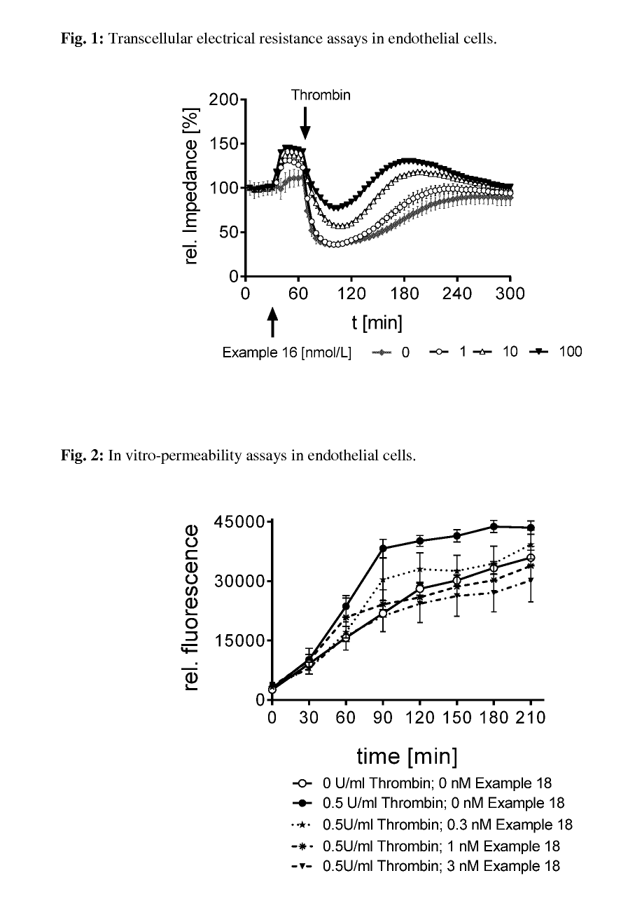

Wild type adrenomedullin (Bachem, H-2932) induces blood pressure reduction in this test with duration of ≦4 h when tested at doses of ≦300 μg/kg body weight (reference WO 2013/064508 A1, FIG. 1). Substances according to the present invention induced blood pressure reduction of up to 8 h at doses of ≦200 μg/kg body weight (as referred to the peptide component).

### A. EXAMPLES

### Adrenomedullin—Analogues

### Abbreviations

AA amino acid
ACN acetonitrile
AcOH acetic acid
ADM adrenomedullin (human)
All allyl
Alloc allyloxycarbonyl
approx. approximately
Boc tert-butyloxycarbonyl
C¹⁶→U²¹/U¹⁶→C²¹ cystathionine
Dab 2,4-diaminobutyric acid
DCM dichloromethane
Dde N-γ-(4,4-dimethyl-2,6-dioxocyclohex-1-ylidene)ethyl
DDTC sodium diethyldithiocarbamate
DIC N,N′-diisopropylcarbodiimide

### DIPEA N,N-diisopropyldiethylamine

### DMF N,N-dimethylformamide

Dpr N-β-4-methyltrityl-L-diaminopropionic acid
EDT ethane-1,2-dithiol
eq. equivalent(s)
ESI electrospray ionization (in MS)
Fmoc N-[(9H-fluoren-9-ylmethoxy)carbonyl
HCl hydrochloric acid
HOBt 1-hydroxybenzotriazole
HPLC high pressure, high performance liquid chromatography
ivDde N-γ-1-(4,4-dimethyl-2,6-dioxocyclohex-1-ylidene)-3-methylbutyl
MALDI matrix-assisted laser desorption/ionization (in MS)

### Mmt Methoxytrityl

MS mass spectrometry

### Mtt Methyltrityl

NaCl sodium chloride
NaOH sodium hydroxyide

### N-Me N-methyl

### NMM N-methylmorpholine

NMP N-methyl-2-pyrrolidone
OPp 2-phenylisopropyl
Oxyma ethyl 2-cyano-2-(hydroxyimino)acetate
PAM palmitic acid
Pbf 2,2,4,6,7-pentamethyldihydrobenzofuran-5-sulfonyl
RP reversed phase (in HPLC)
TA thioanisole
TBST tris buffered saline/Tween® 20
tBu tert-butyl
TFA trifluoroacetic acid
TIS triisopropylsilane
TPP-Pd tetrakis(triphenylphosphine)palladium(0)
Trt trityl
U¹⁶→U²¹ 2,7-diaminosuberic acid
v/v volume/volume

Nomenclature of amino acids and peptide sequences is according to: International Union of Pure and Applied Chemistry and International Union of Biochemistry: Nomenclature and Symbolism for Amino Acids and Peptides (Recommendations 1983). In: Pure & Appl. Chem. 56, Vol. 5, 1984, p. 595-624

| Trivial | | One-letter |
|---|---|---|
| Name | Symbol | Symbol |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Example List

| Ex- | | |
|---|---|---|
| ample | Code | Sequence |
| 1 | [G¹⁴, (E¹⁶, K²¹)_{lac}]ADM(14-52) | H-GGE + RFGTK + TVQKLAHQIYQFTDKDKDNVAPRSKISPQGY-NH₂ |
| 2 | [G¹⁴, (K¹⁶, E²¹)_{lac}]ADM(14-52) | H-GGK + RFGTE + TVQKLAHQIYQFTDKDKDNVAPRSKISPQGY-NH₂ |
| 3 | [G¹⁴, (Dpr¹⁶, D²¹)_{lac}]ADM(14-52) | H-GGDpr + RFGTD + TVQKLAHQIYQFTDKDKDNVAPRSKISPQGY-NH₂ |
| 4 | [G¹⁴, (D¹⁶, Dab²¹)_{lac}]ADM(14-52) | H-GGD + RFGTDab + TVQKLAHQIYQFTDKDKDNVAPRSKISPQGY-NH₂ |
| 5 | [G¹⁴, (Dab¹⁶, D²¹)_{lac}]ADM(14-52) | H-GGDab + RFGTD + TVQKLAHQIYQFTDKDKDNVAPRSKISPQGY-NH₂ |
| 6 | [G¹⁴, (E¹⁶, Dpr²¹)_{lac}]ADM(14-52) | H-GGE + RFGTDpr + TVQKLAHQIYQFTDKDKDNVAPRSKISPQGY-NH₂ |
| 7 | [G¹⁴, (Dpr¹⁶, E²¹)_{lac}]ADM(14-52) | H-GGDpr + RFGTE + TVQKLAHQIYQFTDKDKDNVAPRSKISPQGY-NH₂ |
| 8 | | H-GGD + RFGTOrn + TVQKLAHQIYQFTDKDKDNVAPRSKISPQGY-NH₂ |
| 9 | [G¹⁴, (Orn¹⁶, D²¹)_{lac}]ADM(14-52) | H-GGOrn + RFGTD + TVQKLAHQIYQFTDKDKDNVAPRSKISPQGY-NH₂ |
| 10 | [G¹⁴, (E¹⁶, Dap²¹)_{lac}]ADM(14-52) | H-GGE + RFGTDab + TVQKLAHQIYQFTDKDKDNVAPRSKISPQGY-NH₂ |
| 11 | [G¹⁴, (Dab¹⁶, E²¹)_{lac}]ADM(14-52) | H-GGDab + RFGTE + TVQKLAHQIYQFTDKDKDNVAPRSKISPQGY-NH₂ |
| 12 | [K¹⁴(PAM), (E¹⁶, K²¹)_{lac}]ADM(14-52) | H-K(PAM)GE + RFGTK + TVQKLAHQIYQFTDKDKDNVAPRSKISPQGY-NH₂ |
| 13 | [K¹⁴(PAM), (K¹⁶, E²¹)_{lac}] | H-K(PAM)GK + RFGTE + TVQKLAHQIYQFTDKDKDNVAPRSKISPQGY-NH₂ |
| 14 | [K¹⁴(PAM), (E¹⁶, K²¹)_{lac}]N-Me-I⁴⁷] | H-K(PAM)GE + RFGTK + TVQKLAHQIYQFTDKDKDNVAPRSK-(N-Me)I-SPQGY- |
| | ADM(14-52) | NH₂ |
| 15 | [G¹⁴, (C¹⁶→U²¹)]ADM(14-52) | H-GGC*RFGTU*TVQKLAHQIYQFTDKDKDNVAPRSKISPQGY-NH₂ |
| 16 | [G¹⁴, (U¹⁶→C²¹)]ADM(14-52) | H-GGU*RFGTC*TVQKLAHQIYQFTDKDKDNVAPRSKISPQGY-NH₂ |
| 17 | [G¹⁴, (U¹⁶→U²¹)]ADM(14-52) | H-GGU*RFGTU*TVQKLAHQIYQFTDKDKDNVAPRSKISPQGY-NH₂ |
| 18 | [K¹⁴(PAM), (Dpr¹⁶, E²¹)_{lac}]ADM(14-52) | K(PAM)GDpr + RFGTE + TVQKLAHQIYQFTDKDKDNVAPRSKISPQGY-NH₂ |
| | | (acetate salt) |
| 19 | [G¹⁴, (E¹⁶, Orn²¹)_{lac}]ADM(14-52) | H-GGE + RFGTOrn + TVQKLAHQIYQFTDKDKDNVAPRSKISPQGY-NH₂ |
| 20 | [G¹⁴, (Orn¹⁶, E²¹)_{lac}]ADM(14-52) | H-GGOrn + RFGTE + TVQKLAHQIYQFTDKDKDNVAPRSKISPQGY-NH₂ |
| 21 | [G¹⁴, (K¹⁶, D²¹)_{lac}]ADM(14-52) | H-GGK + RFGTD + TVQKLAHQIYQFTDKDKDNVAPRSKISPQGY-NH₂ |
| 22 | [G¹⁴, (D¹⁶, K²¹)_{lac}]ADM(14-52) | H-GGD + RFGTK + TVQKLAHQIYQFTDKDKDNVAPRSKISPQGY-NH₂ |
| 23 | PAM[G¹⁴, (C¹⁶→U²¹)]ADM(14-52) | PAM-GGC*RFGTU*TVQKLAHQIYQFTDKDKDNVAPRSKISPQGY-NH₂ |
| 24 | PAM[K¹⁴, (C¹⁶→U²¹)]ADM(14-52) | PAM-KGC*RFGTU*TVQKLAHQIYQFTDKDKDNVAPRSKISPQGY-NH₂ |
| 25 | [K¹⁴(PAM), (C¹⁶→U²¹)]ADM(14-52) | H-K(PAM)GC*RFGTU*TVQKLAHQIYQFTDKDKDNVAPRSKISPQGY-NH₂ |
| 26 | [G¹⁴, (D¹⁶, Dpr²¹)_{lac}]ADM(14-52) | H-GGD + RFGTDpr + TVQKLAHQIYQFTDKDKDNVAPRSKISPQGY-NH₂ |
| 27 | [K14(PAM), (Dpr16, E21)lac, N- | H-K(PAM)GDpr + RFGTE + TVQKLAHQIYQFTDKDKDNVAPRS-(N-Me)K- |
| | Me-K46]ADM(14-52) | ISPQGY-NH₂ |

-amino acids in brackets separated with commas (...)_{lac} indicate a lactam-bridge between the side chains of the corresponding amino acids 16 and 21
-amino acids in brackets separated with arrows (...→...) indicate the incorporation of a disulfide bond mimetic of the corresponding amino acids 16 and 21;
-(Pam) indicates the attachment of palmitic acid to the side chain of the corresponding amino acid
-PAM-indicates the attachment of palmitic acid to the N-terminus of the peptide

### Methods

### General Information:

All reactions and procedures were performed at room temperature. After each coupling and deprotection step, the resins were washed with solvent to remove excess of reagents.

### General Method for Peptide Synthesis:

ADM analogues were synthesized stepwise on a NovaSyn®TGR R resin (Novabiochem) with an automated peptide synthesizer (SYRO I, MultiSynTech). The reaction vessels were loaded with 15 μmol NovaSyn®TGR R resin. Each amino acid and the reagents Oxyma and DIC were added in 8-fold molar excess (120 μmol). If not indicated otherwise, the amino acids were N-α-Fmoc-protected; the protecting groups indicated below were used for side chain functionalities. All reactions were performed in DMF. Each coupling step was performed twice with a reaction time of 40 min. Cleavage of the Fmoc protecting group was achieved using 40% piperidine in DMF (v/v) for 3 min and 20% piperidine in DMF (v/v) for 10 min after each coupling step.

### Lactam-Bridged Adrenomedullin—Analogues Examples 1 and 2

### Synthesis:

Examples 1 and 2 were synthesized using the general method described above.

The coupling sequences were as follows:

| | | | AA of |
|---|---|---|---|
| Coupling | | | human |
| Cycle | Example 1 | Example 2 | ADM |
| 1. | Tyr(tBu) | Tyr(tBu) | 52 |
| 2. | Gly | Gly | 51 |
| 3. | Gln(Trt) | Gln(Trt) | 50 |
| 4. | Pro | Pro | 49 |
| 5. | Ser(tBu) | Ser(tBu) | 48 |
| 6. | Ile | Ile | 47 |
| 7. | Lys(Boc) | Lys(Boc) | 46 |
| 8. | Ser(tBu) | Ser(tBu) | 45 |
| 9. | Arg(Pbf) | Arg(Pbf) | 44 |
| 10. | Pro | Pro | 43 |
| 11. | Ala | Ala | 42 |
| 12. | Val | Val | 41 |
| 13. | Asn(Trt) | Asn(Trt) | 40 |
| 14. | Asp(tBu) | Asp(tBu) | 39 |
| 15. | Lys(Boc) | Lys(Boc) | 38 |
| 16. | Asp(tBu) | Asp(tBu) | 37 |
| 17. | Lys(Boc) | Lys(Boc) | 36 |
| 18. | Asp(tBu) | Asp(tBu) | 35 |
| 19. | Thr(tBu) | Thr(tBu) | 34 |
| 20. | Phe | Phe | 33 |
| 21. | Gln(Trt) | Gln(Trt) | 32 |
| 22. | Tyr(tBu) | Tyr(tBu) | 31 |
| 23. | Ile | Ile | 30 |
| 24. | Gln(Trt) | Gln(Trt) | 29 |
| 25. | His(Trt) | His(Trt) | 28 |
| 26. | Ala | Ala | 27 |
| 27. | Leu | Leu | 26 |
| 28. | Lys(Boc) | Lys(Boc) | 25 |
| 29. | Gln(Trt) | Gln(Trt) | 24 |
| 30. | Val | Val | 23 |
| 31. | Thr(tBu) | Thr(tBu) | 22 |
| 32. | Lys(Mmt) | Glu(OPp) | 21 |
| 33. | Thr(tBu) | Thr(tBu) | 20 |
| 34. | Gly | Gly | 19 |
| 35. | Phe | Phe | 18 |
| 36. | Arg(Pbf) | Arg(Pbf) | 17 |
| 37. | | Lys(Mmt) | 16 |
| 38. | | Gly | 15 |

After automated synthesis of the sequence ADM(15-52), for Example 1 the amino acids Fmoc-Glu(OPp) (AA 16) and Fmoc-Gly-OH (AA 15) as well as the N-terminal amino acid Boc-Gly-OH for Examples 1 and 2 were coupled manually with HOBt and DIC in 5-fold molar excess. The reaction was performed in DMF as solvent for 24 h.

Removal of the OPp and Mmt protecting groups was achieved by treatment of the resin (20×2 min) with a cleavage cocktail consisting of TFA/TIS/DCM (1:5:94, v/v/v). Subsequently, the resin was washed (2×5 min) with 5% DIPEA in DMF.

The lactam-bridge was introduced via formation of an amide bond between the side chains of AA 16 and AA 21. The reaction was performed using a 10-fold excess (150 μmol) of HOBt and DIC in DMF as solvent for approx. 24 h.

Cleavage of the peptides from the resin and simultaneous side chain deprotection was achieved with TFA/TIS/H₂O (90:3.5:3.5, v/v/v) for 3 h. The peptides were precipitated and washed with ice-cold diethyl ether, and subsequently lyophilized.

Purification of the crude peptides was performed using preparative RP-HPLC on a C18-column (Phenomenex Jupiter 10 u Proteo 90 Å: 250 mm×21.2 mm, 10 μm, 90 Å). A linear gradient of 10% to 60% eluent B in A over 40 min was applied (Eluent A=0.1% TFA in water; Eluent B=0.08% TFA in ACN). The flow rate was 10 mL/min, UV detection was measured at λ=220 nm.

### Analytics:

The identity of the peptides was confirmed via analytical RP-HPLC, MALDI-MS (UltraflexIII, Bruker) and ESI-MS (HCT, Bruker). The purities were analyzed using analytical RP-HPLC.

### Example 1: [G¹⁴, (E¹⁶,K²¹)_{lac}]ADM(14-52)

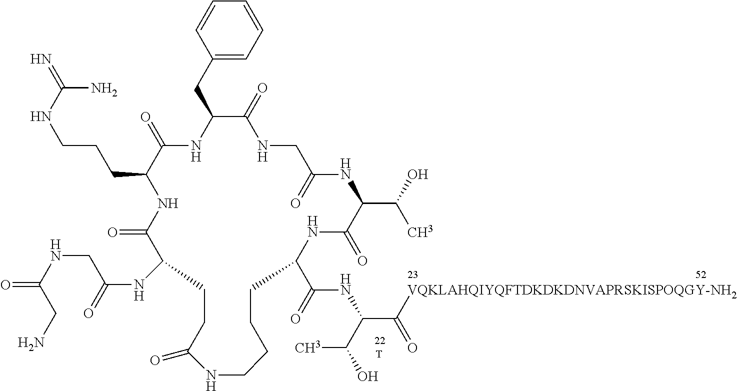

### ((2S,5S,11S,14S,23S)-23-(2-(2-aminoacetamido)acetamido)-5-benzyl-2-(3-guanidinopropyl)-11-((R)-1-hydroxyethyl)-3,6,9,12,20,24-hexaoxo-1,4,7,10,13,19-hexaazacyclotetracosane-14-carbonyl)-L-threonyl-ADM(22-52)

Chemical Formula: C₁₉₅H₃₀₆N₅₈O₅₈

Exact Mass: 4388.278 Da

Molecular Weight: 4390.94 g/mol

Example 1 was synthesized in a 15 μmol scale. The yield was 6.0 mg (9.0% of theory).

Example 1 was analyzed via analytical RP-HPLC using a Jupiter 5 μm C18 300 Å column (Phenomenex, 250 mm×4.6 mm, 5 μm, 300 Å) applying a linear gradient of 10% to 60% eluent B in A over 50 min. Rₜ=30.4 min, purity ≧90%.

In addition, a Jupiter® 4 μm Proteo 90 Å column (Phenomenex, 250 mm×4.6 mm, 4 μm, 90 Å) was used, applying a linear gradient of 10% to 100% eluent B in A over 60 min (Eluent A=0.1% TFA in water; Eluent B=0.08% TFA in ACN; a flow rate=0.6 mL/min; λ=220 nm). Rₜ=21.7 min, purity ≧90%.

The observed mass was in correspondence to the calculated mass. ESI Ion-Trap: m/z=1098.5 [M+4H]⁴⁺, 879.1 [M+5H]⁵⁺, 732.8 [M+6H]⁶⁺, 628.4 [M+7H]⁷⁺, 550.1 [M+8H]⁸⁺; MALDI-TOF: m/z=4389.4 [M+H]⁺, 2195.1 [M+2H]²⁺.

### Example 2: [G¹⁴, (K¹⁶,E²¹)_{lac}]ADM(14-52)

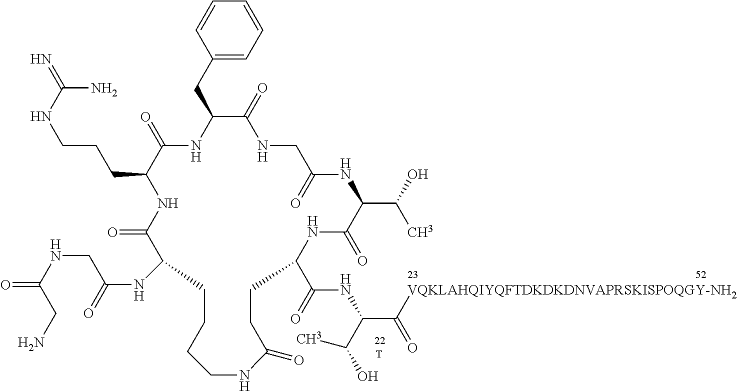

### ((2S,5S,11S,14S,23S)-23-(2-(2-aminoacetamido)acetamido)-5-benzyl-2-(3-guanidinopropyl)-11-((R)-1-hydroxyethyl)-3,6,9,12,17,24-hexaoxo-1,4,7,10,13,18-hexaazacyclotetracosane-14-carbonyl)-L-threonyl-ADM(22-52)

Chemical Formula: C₁₉₅H₃₀₆N₅₈O₅₈

Exact Mass: 4388.278 Da

Molecular Weight: 4390.94 g/mol

Example 2 was synthesized in a 15 μmol scale. The yield was 3.8 mg (5.8% of theory)

Example 2 was analyzed via analytical RP-HPLC using a Jupiter® 5 μm C18 300 Å column (Phenomenex, 250 mm×4.6 mm, 5 μm, 300 Å) applying a linear gradient of 10% to 60% eluent B in A over 50 min (Eluent A=0.1% TFA in water; Eluent B=0.08% TFA in ACN; flow rate of 0.6 mL/min. Rₜ=30.1 min, purity ≧90%.

In addition, a Kinetex® 5 μm XB-C18 column (Phenomenex, 250×4.6 mm, 5 μm, 100 Å) was used, applying a linear gradient of 10% to 60% eluent B in A over 50 min (Eluent A=0.1% TFA in water; Eluent B=0.08% TFA in ACN; flow rate=1.25 mL/min; λ=220 nm). Rₜ=23.0 min, purity ≧90%.

The observed mass was in correspondence to the calculated mass. ESI Ion-Trap: m/z=1098.5 [M+4H]⁴⁺, 879.1 [M+5H]⁵⁺, 732.8 [M+6H]⁶⁺, 628.4 [M+7H]⁷⁺, 547.9 [M+8H]⁸⁺; MALDI-TOF: m/z=4389.4 [M+H]⁺, 2195.0 [M+2H]²⁺, 1464.0 [M+3H]³⁺.

### Lactam-Bridged Adrenomedullin—Analogues Examples 3-11, 19-22, and 26

### Synthesis:

The syntheses of Examples 3-8, 10, 11, and 19-22 were performed using automated peptide synthesis of the sequence ADM(22-52) as described in the general method. Subsequently, positions 21 to 14 were incorporated manually. The sequences [G¹⁴, Orn¹⁶(ivDde), D²¹(OPp)]ADM(14-52) of Example 9 and [G¹⁴, D¹⁶(OPp), Dpr²¹(Mtt)]ADM(14-52) of Example 26 were synthesized in an automated manner as described in the general method. The N-terminus of all compounds was protected with Fmoc, except for examples 9 and 26, where Boc Gly-OH was incorporated as terminal amino acid.

The coupling sequence of ADM(22-52) was as follows:

| | | AA of |
|---|---|---|
| Coupling | ADM | human |
| Cycle | (22-52) | ADM |
| 1. | Tyr(tBu) | 52 |
| 2. | Gly | 51 |
| 3. | Gln(Trt) | 50 |
| 4. | Pro | 49 |
| 5. | Ser(tBu) | 48 |
| 6. | Ile | 47 |
| 7. | Lys(Boc) | 46 |
| 8. | Ser(tBu) | 45 |
| 9. | Arg(Pbf) | 44 |
| 10. | Pro | 43 |
| 11. | Ala | 42 |
| 12. | Val | 41 |
| 13. | Asn(Trt) | 40 |
| 14. | Asp(tBu) | 39 |
| 15. | Lys(Boc) | 38 |
| 16. | Asp(tBu) | 37 |
| 17. | Lys(Boc) | 36 |
| 18. | Asp(tBu) | 35 |
| 19. | Thr(tBu) | 34 |
| 20. | Phe | 33 |
| 21. | Gln(Trt) | 32 |
| 22. | Tyr(tBu) | 31 |
| 23. | Ile | 30 |
| 24. | Gln(Trt) | 29 |
| 25. | His(Trt) | 28 |
| 26. | Ala | 27 |
| 27. | Leu | 26 |
| 28. | Lys(Boc) | 25 |
| 29. | Gln(Trt) | 24 |
| 30. | Val | 23 |
| 31. | Thr(tBu) | 22 |

The coupling sequences of compounds 9 and 26 were as follows:

| | | AA of |
|---|---|---|
| Coupling | | human |
| Cycle | Example 9 | ADM |
| 1. | Tyr(tBu) | 52 |
| 2. | Gly | 51 |
| 3. | Gln(Trt) | 50 |
| 4. | Pro | 49 |
| 5. | Ser(tBu) | 48 |
| 6. | Ile | 47 |
| 7. | Lys(Boc) | 46 |
| 8. | Ser(tBu) | 45 |
| 9. | Arg(Pbf) | 44 |
| 10. | Pro | 43 |
| 11. | Ala | 42 |
| 12. | Val | 41 |
| 13. | Asn(Trt) | 40 |
| 14. | Asp(tBu) | 39 |
| 15. | Lys(Boc) | 38 |
| 16. | Asp(tBu) | 373 |
| 17. | Lys(Boc) | 36 |
| 18. | Asp(tBu) | 35 |
| 19. | Thr(tBu) | 34 |
| 20. | Phe | 33 |
| 21. | Gln(Trt) | 32 |
| 22. | Tyr(tBu) | 31 |
| 23. | Ile | 30 |
| 24. | Gln(Trt) | 29 |
| 25. | His(Trt) | 28 |
| 26. | Ala | 27 |
| 27. | Leu | 26 |
| 28. | Lys(Boc) | 25 |
| 29. | Gln(Trt) | 24 |
| 30. | Val | 23 |
| 31. | Thr(tBu) | 22 |
| 32. | Asp(PP) | 21 |
| 33. | Thr(tBu) | 20 |
| 34. | Gly | 19 |
| 35. | Phe | 18 |
| 36. | Arg(Pbf) | 17 |
| 37. | Orn(ivDde) | 16 |
| 38. | Gly | 15 |
| 39. | Boc-Gly-OH | 14 |

Amino acid 21 of compounds 3-8, 10, 11 and 19-22 (see table below) was coupled manually using a 5-fold molar excess of amino acid, HOBt and DIC in DMF as solvent for approx. 24 h.

Subsequent Fmoc-deprotection was achieved by treatment of the resin with 20% piperidine in DMF (v/v) twice for 10 min.

| Example | AA 21 |
|---|---|
| 3 | Fmoc-Asp(OPp)-OH |
| 4 | Fmoc-Dab(Dde)-OH |
| 5 | Fmoc-Asp(OPp)-OH |
| 6 | Fmoc-Dpr(ivDde)-OH |
| 7 | Fmoc-Glu(OPp)-OH |
| 8 | Fmoc-Orn(ivDde)-OH |
| 10 | Fmoc-Dab(Dde)-OH |
| 11 | Fmoc-Glu(OPp)-OH |
| 19 | Fmoc-Orn(ivDde)-OH |
| 20 | Fmoc-Glu(OPp)-OH |
| 21 | Fmoc-Asp(OPp)-OH |
| 22 | Fmoc-Lys(Mmt)-OH |

The following four amino acids of compounds 3-8, 10, 11 and 19-22 were coupled manually using a 5-fold molar excess of amino acid, HOBt and DIC in DMF as solvent for approx. 24 h. Fmoc-deprotection after the first three couplings was achieved by treatment of the resin with 20% piperidine in DMF (v/v) twice for 10 min.

The coupling sequence was as follows:

| | | AA of |
|---|---|---|
| Coupling | Examples 3-8, | human |
| Cycle | 10 and 11 | ADM |
| 1. | Fmoc-Thr(tBu)-OH | 20 |
| 2. | Fmoc-Gly-OH | 19 |
| 3. | Fmoc-Phe-OH | 18 |
| 4. | Fmoc-Arg(Pbf)-OH | 17 |

Amino acid 16 of Examples 3-8, 10, 11 and 19-22 (see table below) was coupled manually using a 5-fold molar excess of amino acid, HOBt and DIC in DMF as solvent for approx. 24 h. Subsequent Fmoc-deprotection was achieved by treatment of the resin with 20% piperidine in DMF (v/v) twice for 10 min.

| Example | AA 16 |
|---|---|
| 3 | Fmoc-Dpr(Mtt)-OH |
| 4 | Fmoc-Asp(PP)-OH |
| 5 | Fmoc-Dab(Dde)-OH |
| 6 | Fmoc-Glu(PP)-OH |
| 7 | Fmoc-Dpr(Mtt)-OH |
| 8 | Fmoc-Asp(PP)-OH |
| 10 | Fmoc-Glu(PP)-OH |
| 11 | Fmoc-Dab(Dde)-OH |
| 19 | Fmoc-Glu(OPp)-OH |
| 20 | Fmoc-Orn(ivDde)-OH |
| 21 | Fmoc-Lys(Mmt)-OH |
| 22 | Fmoc-Asp(OPp)-OH |

The following two amino acids of compounds 3-8, 10, 11 and 19-22 were coupled manually using a 5-fold molar excess of amino acid, HOBt and DIC in DMF as solvent for approx. 24 h. Fmoc-deprotection after the first coupling was achieved by treatment of the resin with 20% piperidine in DMF (v/v) twice for 5 min.

The coupling sequence was as follows:

| | | AA of |
|---|---|---|
| Coupling | Examples 3-8, | human |
| Cycle | 10 and 11 | ADM |
| 1. | Fmoc-Gly-OH | 15 |
| 2. | Boc-Gly-OH | 14 |

For simultaneous removal of Dde/ivDde protecting groups the resins of compounds 4-6 and 8-11, 19 and 20 were treated with 3% hydrazine monohydrate in DMF (v/v) (15×10 min, 1 mL).

The following steps were applied for the synthesis of compounds 3-11, 19-22 and 26.

For simultaneous removal of Mmt/OPp protecting groups the resins were treated with TFA/TIS/DCM (2:5:93, v/v/v) (15×2 min, 1 mL). Subsequently, the resins were washed with 2% DIPEA in DMF (v/v) twice for 10 min (1 mL).

Cyclization was performed using a 6-fold excess of HOBt and DIC in DMF as solvent for approx. 24 h.

Cleavage of the peptides from the resin and simultaneous side chain deprotection was achieved with TFA/TA/EDT (90:7:3, v/v/v) for approx. 3 h. The peptides were precipitated and washed with ice-cold diethyl ether/n-hexane (4/1; v/v) and subsequently lyophilized.

Purification of the compounds 3-11, 19 and 20 was performed using preparative RP-HPLC on a C18-column (XBridge BEH130 Prep C18 10 μm OBD: 250 mm×19 mm, 10 μm). A linear gradient of 10% to 45% eluent B in A over 30 min was applied (Eluent A=0.1% TFA in water; Eluent B=0.08% TFA in ACN). The flow rate was 20 mL/min, UV detection was measured at λ=220 nm.

Purification of the compounds 21, 22, and 26 was performed using preparative RP-HPLC on a C18-column (Kinetex® 5 μm XB-C18 100 Å: 250 mm×21.2 mm, 5 μm). A linear gradient of 10% to 45% eluent B in A over 30 min was applied (Eluent A=0.1% TFA in water; Eluent B=0.08% TFA in ACN). The flow rate was 20 mL/min, UV detection was measured at λ=220 nm.

### Analytics:

The identity of the peptides was confirmed via MALDI-MS (UltraflexIII, Bruker) and ESI-MS (HCT, Bruker). The purities were analyzed using analytical RP-HPLC.

### Example 3: [G¹⁴, (Dpr¹⁶, D²¹)_{lac}]ADM(14-52)

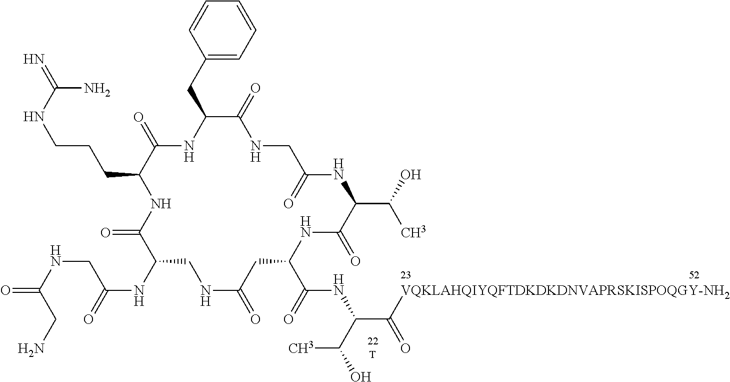

### ((2S,5S,11S,14S,19S)-19-(2-(2-aminoacetamido)acetamido)-5-benzyl-2-(3-guanidinopropyl)-11-((R)-1-hydroxyethyl)-3,6,9,12,16,20-hexaoxo-1,4,7,10,13,17-hexaazacycloicosane-14-carbonyl)-L-threonyl-ADM(22-52)

Chemical Formula: C₁₉₁H₂₉₈N₅₈O₅₈

Exact Mass: 4332.215 Da

Molecular Weight: 4334.83 g/mol

Example 3 was synthesized in a 15 μmol scale. The yield was 1.9 mg (2.9% of theory).

Example 3 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 50 min (Eluent A=0.1% TFA in water; Eluent B=0.08% TFA in ACN; flow rate=0.6 mL/min; λ=220 nm). Rt=27.8 min, purity ≧95%

In addition, a Jupiter® 5 μm C18 300 Å column (Phenomenex, 250 mm×4.6 mm, 5 μm, 300 Å) was used, applying a linear gradient of 10% to 60% eluent B in A over 50 min (Eluent A=0.1% TFA in water; Eluent B=0.08% TFA in ACN; flow rate=0.6 mL/min; λ=220 nm). Rt=31.3 min, purity ≧95%

The observed mass was in correspondence to the calculated mass. ESI Ion-Trap: m/z=1084.5 [M+4H]⁴⁺, 867.8 [M+5H]⁵⁺, 723.5 [M+6H]⁶⁺, 620.3 [M+7H]⁷⁺, 542.8 [M+8H]⁸⁺; MALDI-TOF: m/z=4333.2 [M+H]⁺, 2167.1 [M+2H]²⁺.

### Example 4: [G¹⁴, (D¹⁶, Dab²¹)_{lac}]ADM(14-52)

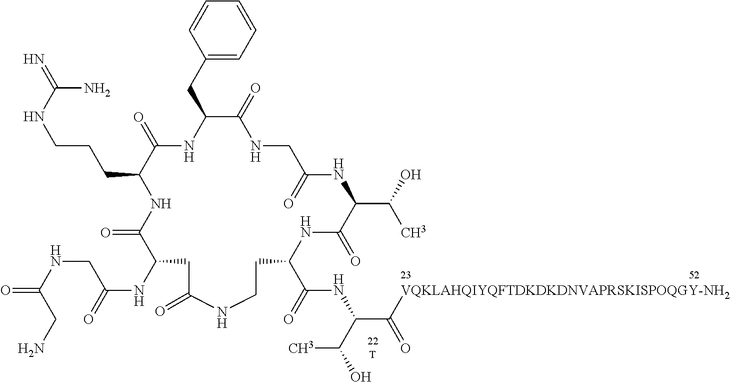

### ((2S,5S,11S,14S,20S)-20-(2-(2-aminoacetamido)acetamido)-5-benzyl-2-(3-guanidinopropyl)-11-((R)-1-hydroxyethyl)-3,6,9,12,18,21-hexaoxo-1,4,7,10,13,17-hexaazacyclohenicosane-14-carbonyl)-L-threonyl-ADM(22-52)

Chemical Formula: C₁₉₂H₃₀₀N₅₈O₅₈

Exact Mass: 4346.231 Da

Molecular Weight: 4348.86 g/mol

Example 4 was synthesized in a 15 μmol scale. The yield was 1.8 mg (2.6% of theory).

Example 4 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 50 min (Eluent A=0.1% TFA in water; Eluent B=0.08% TFA in ACN; flow rate=0.6 mL/min; λ=220 nm). Rt=26.3 min, purity ≧95%.

In addition, a Jupiter® 5 μm C18 300 Å column (Phenomenex, 250 mm×4.6 mm, 5 μm, 300 Å) was used, applying a linear gradient of 10% to 60% eluent B in A over 50 min (Eluent A=0.1% TFA in water; Eluent B=0.08% TFA in ACN; flow rate=0.6 mL/min; λ=220 nm). Rt=29.5 min, purity ≧95%.

The observed mass was in correspondence to the calculated mass. ESI Ion-Trap: m/z=1088.3 [M+4H]⁴⁺, 870.7 [M+5H]⁵⁺, 725.9 [M+6H]⁶⁺, 622.2 [M+7H]⁷⁺, 544.5 [M+8H]⁸⁺; MALDI-TOF: m/z=4347.2 [M+H]⁺, 2174.1[M+2H]²⁺.

### Example 5: [G¹⁴, (Dab¹⁶, D²¹)_{lac}]ADM(14-52)

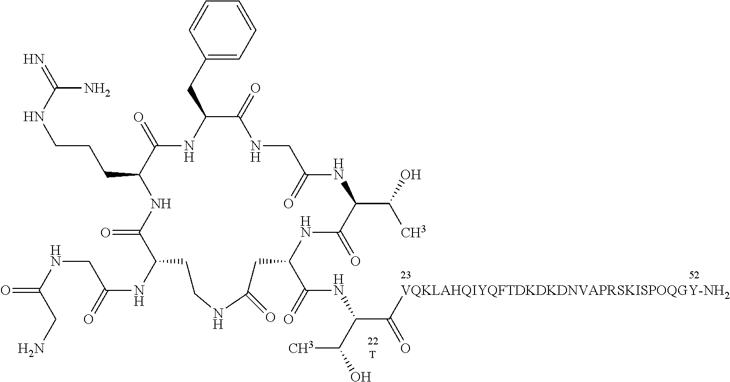

### ((2S,5S,11S,14S,20S)-20-(2-(2-aminoacetamido)acetamido)-5-benzyl-2-(3-guanidinopropyl)-11-((R)-1-hydroxyethyl)-3,6,9,12,16,21-hexaoxo-1,4,7,10,13,17-hexaazacyclohenicosane-14-carbonyl)-L-threonyl-ADM(22-52)

Chemical Formula: C₁₉₂H₃₀₀N₅₈O₅₈

Exact Mass: 4346.231 Da

Molecular Weight: 4348.86 g/mol

Example 5 was synthesized in a 15 μmol scale. The yield was 1.4 mg (1.9% of theory).

Example 5 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 50 min (Eluent A=0.1% TFA in water; Eluent B=0.08% TFA in ACN; flow rate=0.6 mL/min; λ=220 nm). Rt=26.1 min, purity ≧90%

In addition, a Jupiter® 5 μm C18 300 Å column (Phenomenex, 250 mm×4.6 mm, 5 μm, 300 Å) was used, applying a linear gradient of 10% to 60% eluent B in A over 50 min (Eluent A=0.1% TFA in water; Eluent B=0.08% TFA in ACN; flow rate=0.6 mL/min; λ=220 nm). Rt=29.1 min, purity ≧90%.

The observed mass was in correspondence to the calculated mass. ESI Ion-Trap: m/z=1088.1 [M+4H]⁴⁺, 870.7 [M+5H]⁵⁺, 725.8 [M+6H]⁶⁺, 622.2 [M+7H]⁷⁺, 544.5 [M+8H]⁸⁺; MALDI-TOF: m/z=4347.2 [M+H]⁺, 2174.1[M+2H]²⁺.

### Example 6: [G¹⁴, (E¹⁶, Dpr²¹)_{lac}]ADM(14-52)

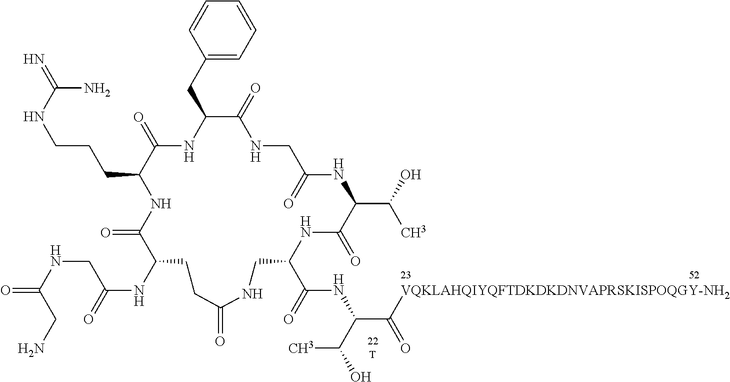

### ((3S,6S,12S,15S,18S)-18-(2-(2-aminoacetamido)acetamido)-12-benzyl-15-(3-guanidinopropyl)-6-((R)-1-hydroxyethyl)-5,8,11,14,17,21-hexaoxo-1,4,7,10,13,16-hexaazacyclohenicosane-3-carbonyl)-L-threonyl-ADM(22-52)

Chemical Formula: C₁₉₂H₃₀₀N₅₈O₅₈

Exact Mass: 4346.231 Da

Molecular Weight: 4348.86 g/mol

Example 6 was synthesized in a 15 μmol scale. The yield was 1.2 mg (1.7% of theory).

Example 6 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.9 min, purity ≧95%.

In addition, a Jupiter® 5 μm C18 300 Å column (Phenomenex, 250 mm×4.6 mm, 5 μm, 300 Å) was used, applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.6 min, purity ≧95%.

The observed mass was in correspondence to the calculated mass ESI Ion-Trap: m/z=1088.3 [M+4H]⁴⁺, 870.6 [M+5H]⁵⁺, 725.7 [M+6H]⁶⁺, 622.2 [M+7H]⁷⁺, 544.5 [M+8H]⁸⁺; MALDI-TOF: m/z=4347.2 [M+H]⁺, 2174.1[M+2H]²⁺.

### Example 7: [G¹⁴, (Dpr¹⁶, E²¹)_{lac}]ADM(14-52)

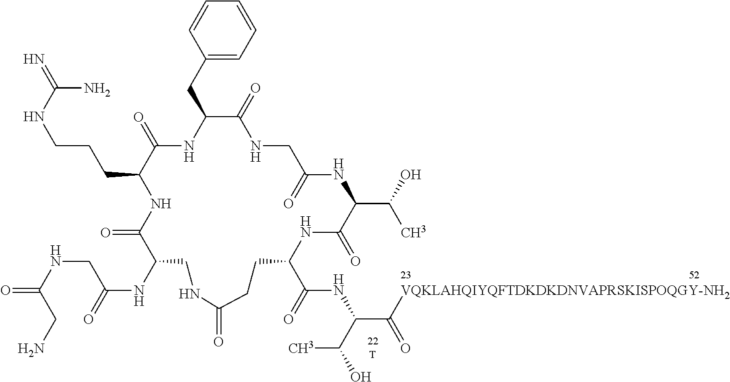

### ((3S,9S,12S,15S,21S)-15-(2-(2-aminoacetamido)acetamido)-9-benzyl-12-(3-guanidinopropyl)-3-((R)-1-hydroxyethyl)-2,5,8,11,14,18-hexaoxo-1,4,7,10,13,17-hexaazacyclohenicosane-21-carbonyl)-L-threonyl-ADM(22-52)

Chemical Formula: C₁₉₂H₃₀₀N₅₈O₅₈

Exact Mass: 4346.231 Da

Molecular Weight: 4348.86 g/mol

Example 7 was synthesized in a 15 μmol scale. The yield was 1.8 mg (2.7% of theory).

Example 7 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 40 min (Eluent A=0.1% TFA in water; Eluent B=0.08% TFA in ACN; flow rate=0.6 mL/min; λ=220 nm). Rt=23.4 min, purity ≧95%.

In addition, a Jupiter® 5 μm C18 300 Å column (Phenomenex, 250 mm×4.6 mm, 5 μm, 300 Å) was used, applying a linear gradient of 10% to 60% eluent B in A over 40 min (Eluent A=0.1% TFA in water; Eluent B=0.08% TFA in ACN; flow rate=0.6 mL/min; λ=220 nm). Rt=23.2 min, purity ≧95%.

The observed mass was in correspondence to the calculated mass ESI Ion-Trap: m/z=1088.6 [M+4H]⁴⁺, 870.6 [M+5H]⁵⁺, 725.7 [M+6H]⁶⁺, 622.2 [M+7H]⁷⁺, 544.5 [M+8H]⁸⁺; MALDI-TOF: m/z=4347.2 [M+H]⁺, 2174.0[M+2H]²⁺.

### Example 8: [G¹⁴, (D¹⁶, Orn²¹)_{lac}]ADM(14-52)

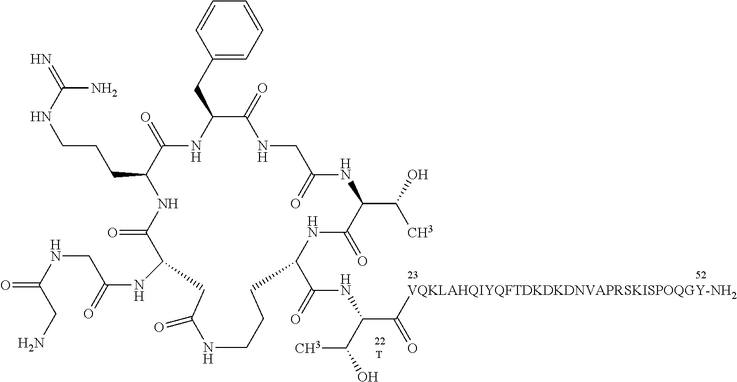

### ((2S,5S,11S,14S,21S)-21-(2-(2-aminoacetamido)acetamido)-5-benzyl-2-(3-guanidinopropyl)-11-((R)-1-hydroxyethyl)-3,6,9,12,19,22-hexaoxo-1,4,7,10,13,18-hexaazacyclodocosane-14-carbonyl)-L-threonyl-ADM(22-52)

Chemical Formula: C₁₉₃H₃₀₂N₅₈O₅₈

Exact Mass: 4360.247 Da

Molecular Weight: 4362.89 g/mol

Example 8 was synthesized in a 15 μmol scale. The yield was 2.4 mg (3.7% of theory, purity ≧95%).

Example 8 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 40 min (Eluent A=0.1% TFA in water; Eluent B=0.08% TFA in ACN; flow rate=0.6 mL/min; λ=220 nm). Rt=23.1 min, purity ≧95%.

In addition, a Jupiter® 5 μm C18 300 Å column (Phenomenex, 250 mm×4.6 mm, 5 μm, 300 Å) was used, applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.8 min, purity ≧95%.

The observed mass was in correspondence to the calculated mass ESI Ion-Trap: m/z=1091.5 [M+4H]⁴⁺, 873.8 [M+5H]⁵⁺, 728.1 [M+6H]⁶⁺, 624.3 [M+7H]⁷⁺, 546.3 [M+8H]⁸⁺; MALDI-TOF: m/z=4361.3 [M+H]⁺, 2181.1 [M+2H]²⁺.

### Example 9: [G¹⁴, (Orn¹⁶, D²¹)_{lac}]ADM(14-52)

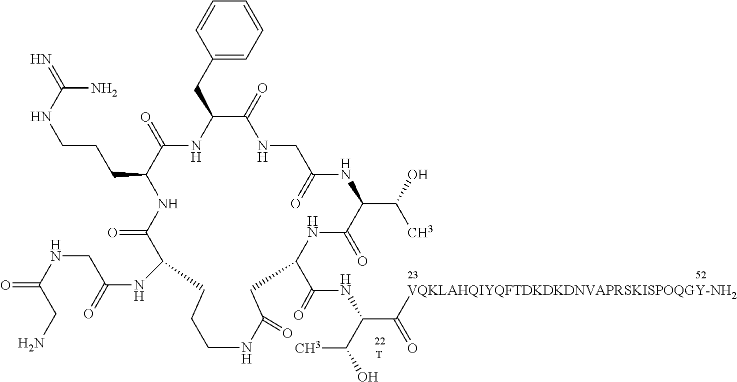

((2S,5S,11S,14S,21S)-21-(2-(2-aminoacetamido)acetamido)-5-benzyl-2-(3-guanidinopropyl)-11-((R)-1-hydroxyethyl)-3,6,9,12,16,22-hexaoxo-1,4,7,10,13,17-hexaazacyclodocosane-14-carbonyl)-L-threonyl-ADM(22-52)

Chemical Formula: C₁₉₃H₃₀₂N₅₈O₅₈

Exact Mass: 4360.247 Da

Molecular Weight: 4362.89 g/mol

Example 9 was synthesized in a 15 μmol scale. The yield was 7.9 mg (12.1% of theory).

Example 9 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 40 min (Eluent A=0.1% TFA in water; Eluent B=0.08% TFA in ACN; flow rate=0.6 mL/min; λ=220 nm). Rt=23.1 min, purity ≧95%.

In addition, a Jupiter® 5 μm C18 300 Å column (Phenomenex, 250 mm×4.6 mm, 5 μm, 300 Å) was used, applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.9 min, purity ≧95%.

The observed mass was in correspondence to the calculated mass ESI Ion-Trap: m/z=1091.7 [M+4H]⁴⁺, 873.5 [M+5H]⁵⁺, 728.1 [M+6H]⁶⁺, 624.3 [M+7H]⁷⁺, 546.3 [M+8H]⁸⁺; MALDI-TOF: m/z=4361.2 [M+H]⁺, 2181.0 [M+2H]²⁺.

### Example 10: [G¹⁴, (E¹⁶, Dab²¹)_{lac}]ADM(14-52)

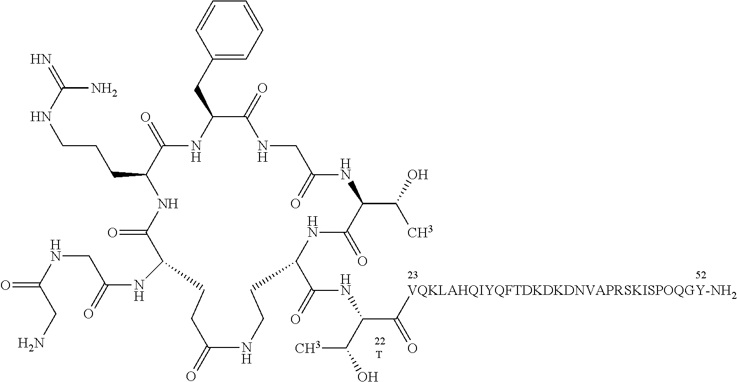

### ((2S,5S,11S,14S,21S)-21-(2-(2-aminoacetamido)acetamido)-5-benzyl-2-(3-guanidinopropyl)-11-((R)-1-hydroxyethyl)-3,6,9,12,18,22-hexaoxo-1,4,7,10,13,17-hexaazacyclodocosane-14-carbonyl)-L-threonyl-ADM(22-52)

Chemical Formula: C₁₉₃H₃₀₂N₅₈O₅₈

Exact Mass: 4360.247 u

Molecular Weight: 4362.89 g/mol

Example 10 was synthesized in a 15 μmol scale. The yield was 1.9 mg (3.0% of theory).

Example 10 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.5 min, purity ≧95%.

In addition, a Jupiter® 5 μm C18 300 Å column (Phenomenex, 250 mm×4.6 mm, 5 μm, 300 Å) was used, applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.3 min, purity ≧95%.

The observed mass was in correspondence to the calculated mass ESI Ion-Trap: m/z=1091.7 [M+4H]⁴⁺, 873.6 [M+5H]⁵⁺, 728.1 [M+6H]⁶⁺, 624.3 [M+7H]⁷⁺, 546.3 [M+8H]⁸⁺; MALDI-TOF: m/z=4361.2 [M+H]⁺, 2181.0 [M+2H]²⁺.

### Example 11: [G¹⁴, (Dab¹⁶,E²¹)_{lac}]ADM(14-52)

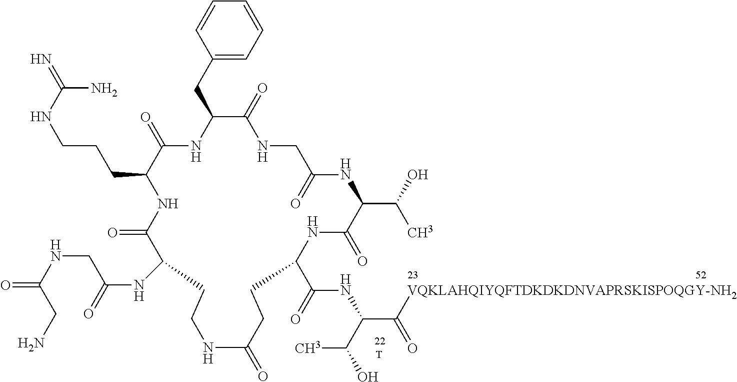

### ((2S,5S,11S,14S,21S)-21-(2-(2-aminoacetamido)acetamido)-5-benzyl-2-(3-guanidinopropyl)-11-((R)-1-hydroxyethyl)-3,6,9,12,17,22-hexaoxo-1,4,7,10,13,18-hexaazacyclodocosane-14-carbonyl)-L-threonyl-ADM(22-52)

Chemical Formula: C₁₉₃H₃₀₂N₅₈O₅₈

Exact Mass: 4360.247 Da

Molecular Weight: 4362.89 g/mol

Example 11 was synthesized in a 15 μmol scale. The yield was 1.5 mg (2.3% of theory).

Example 11 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.8 min, purity ≧95%.

In addition, a Jupiter® 5 μm C18 300 Å column (Phenomenex, 250 mm×4.6 mm, 5 μm, 300 Å) was used, applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.6 min, purity ≧95%.

The observed mass was in correspondence to the calculated mass ESI Ion-Trap: m/z=1091.9 [M+4H]⁴⁺, 873.5 [M+5H]⁵⁺, 728.1 [M+6H]⁶⁺, 624.3 [M+7H]⁷⁺, 546.3 [M+8H]⁸⁺; MALDI-TOF: m/z=4361.2 [M+H]⁺, 2181.0 [M+2H]²⁺.

### Example 19: [G¹⁴, (E¹⁶,Orn²¹)_{lac}]ADM(14-52)

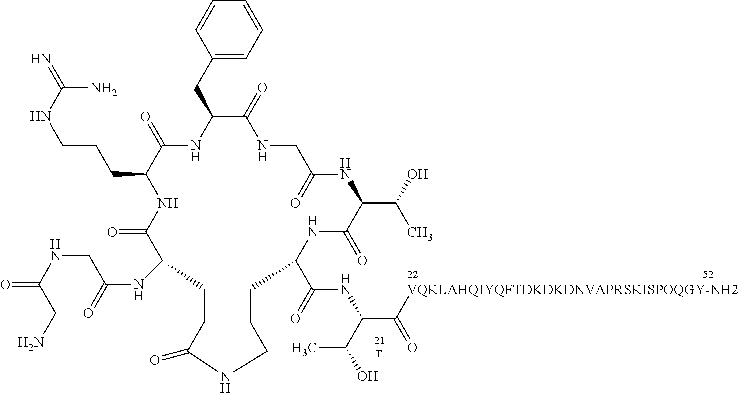

### ((2S,5S,11S,14S,22S)-22-(2-(2-aminoacetamido)acetamido)-5-benzyl-2-(3-guanidinopropyl)-11-((R)-1-hydroxyethyl)-3,6,9,12,19,23-hexaoxo-1,4,7,10,13,18-hexaazacyclotricosane-14-carbonyl)-L-threonyl-ADM(22-52)

Chemical Formula: C₁₉₄H₃₀₄N₅₈O₅₈

Exact Mass: 4374.26 Da

Molecular Weight: 4376.91 g/mol

Example 19 was synthesized in a 15 μmol scale. The yield was 1.6 mg (2.4% of theory).

Example 19 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 40 min (Eluent A=0.1% TFA in water; Eluent B=0.08% TFA in ACN; flow rate=0.6 mL/min; λ=220 nm). Rt=23.2 min, purity ≧95%

In addition, a Jupiter® 5 μm C18 300 Å column (Phenomenex, 250 mm×4.6 mm, 5 μm, 300 Å) was used, applying a linear gradient of 10% to 60% eluent B in A over 40 min (Eluent A=0.1% TFA in water; Eluent B=0.08% TFA in ACN; flow rate=0.6 mL/min; λ=220 nm). Rt=23.2 min, purity ≧95%

The observed mass was in correspondence to the calculated mass ESI Ion-Trap: m/z=1095.4 [M+4H]⁴⁺, 876.4 [M+5H]⁵⁺, 730.5 [M+6H]⁶⁺, 626.3 [M+7H]⁷⁺, 548.1 [M+8H]⁸⁺; MALDI-TOF: m/z=4375.3 [M+H]⁺, 2188.0 [M+2H]²⁺.

### Example 20: [G¹⁴, (Orn¹⁶,E²¹)_{lac}]ADM(14-52)

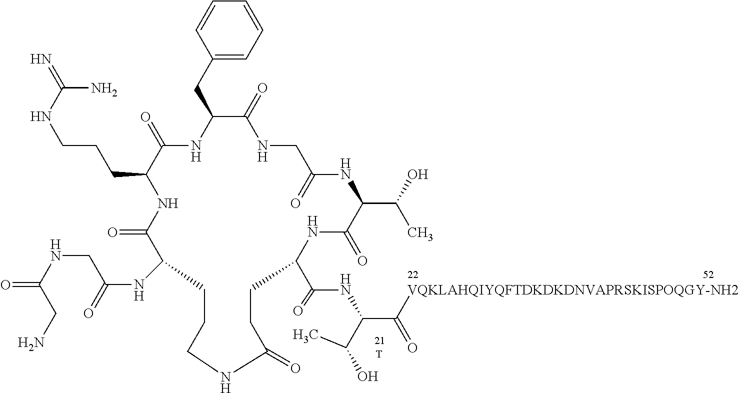

### ((2S,5S,11S,14S,22S)-22-(2-(2-aminoacetamido)acetamido)-5-benzyl-2-(3-guanidinopropyl)-11-((R)-1-hydroxyethyl)-3,6,9,12,17,23-hexaoxo-1,4,7,10,13,18-hexaazacyclotricosane-14-carbonyl)-L-threonyl-ADM(22-52)

Chemical Formula: C₁₉₄H₃₀₄N₅₈O₅₈

Exact Mass: 4374.26 Da

Molecular Weight: 4376.91 g/mol

Example 20 was synthesized in a 15 μmol scale. The yield was 1.7 mg (2.6% of theory).

Example 20 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.8 min, purity ≧95%

In addition, a Jupiter® 5 μm C18 300 Å column (Phenomenex, 250 mm×4.6 mm, 5 μm, 300 Å) was used, applying a linear gradient of 10% to 60% eluent B in A over 40 min (Eluent A=0.1% TFA in water; Eluent B=0.08% TFA in ACN; flow rate=0.6 mL/min; λ=220 nm). Rt=22.9 min, purity ≧95%

The observed mass was in correspondence to the calculated mass ESI Ion-Trap: m/z=1095.4 [M+4H]⁴⁺, 876.3 [M+5H]⁵⁺, 730.5 [M+6H]⁶⁺, 626.3 [M+7H]⁷⁺, 548.1 [M+8H]⁸⁺; MALDI-TOF: m/z=4375.2 [M+H]⁺, 2188.0 [M+2H]²⁺.

### Example 21: [G¹⁴, (K¹⁶,D²¹)_{lac}]ADM(14-52)

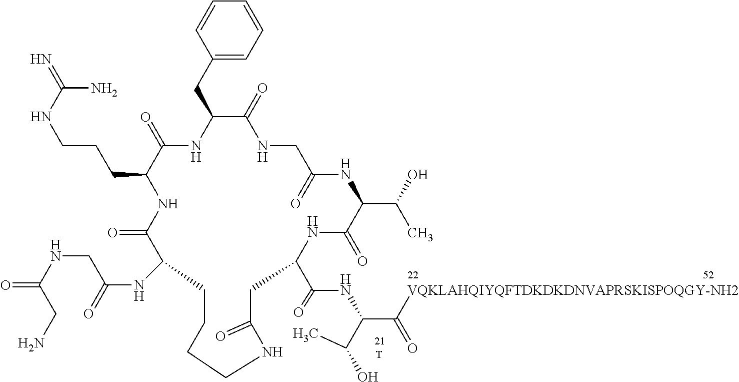

### ((2S,5S,11S,14S,22S)-22-(2-(2-aminoacetamido)acetamido)-5-benzyl-2-(3-guanidinopropyl)-11-((R)-1-hydroxyethyl)-3,6,9,12,16,23-hexaoxo-1,4,7,10,13,17-hexaazacyclotricosane-14-carbonyl)-L-threonyl-ADM(22-52)

Chemical Formula: C₁₉₄H₃₀₄N₅₈O₅₈

Exact Mass: 4374.26 Da

Molecular Weight: 4376.91 g/mol

Example 21 was synthesized in a 15 μmol scale. The yield was 0.3 mg (0.5% of theory).

Example 21 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 50 min (Eluent A=0.1% TFA in water; Eluent B=0.08% TFA in ACN; flow rate=0.6 mL/min; λ=220 nm). Rt=26.1 min, purity ≧95%

In addition, a Jupiter® 5 μm C18 300 Å column (Phenomenex, 250 mm×4.6 mm, 5 μm, 300 Å) was used, applying a linear gradient of 10% to 60% eluent B in A over 50 min (Eluent A=0.1% TFA in water; Eluent B=0.08% TFA in ACN; flow rate=0.6 mL/min; λ=220 nm). Rt=25.9 min, purity ≧95%

The observed mass was in correspondence to the calculated mass ESI Ion-Trap: m/z=1095.5 [M+4H]⁴⁺, 876.3 [M+5H]⁵⁺, 730.5 [M+6H]⁶⁺, 626.3 [M+7H]⁷⁺, 548.1 [M+8H]⁸⁺; MALDI-TOF: m/z=4375.2 [M+H]⁺, 2188.1 [M+2H]²⁺, 1459.1 [M+3H]³⁺.

### Example 22: [G¹⁴, (D¹⁶,K²¹)_{lac}]ADM(14-52)

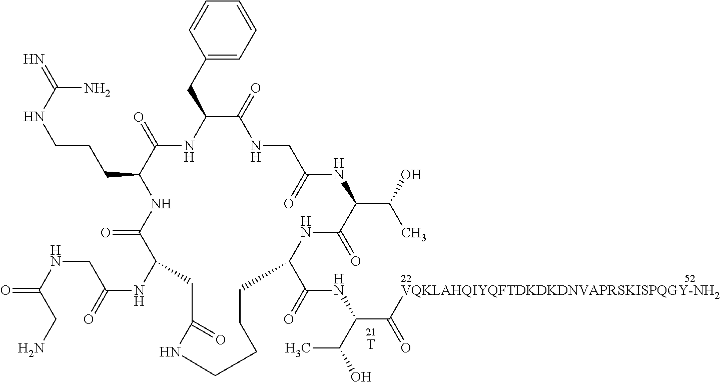

### ((3S,9S,12S,15S,23S)-15-(2-(2-aminoacetamido)acetamido)-9-benzyl-12-(3-guanidinopropyl)-3-((R)-1-hydroxyethyl)-2,5,8,11,14,17-hexaoxo-1,4,7,10,13,18-hexaazacyclotricosane-23-carbonyl)-L-threonyl-ADM(22-52)

Chemical Formula: C₁₉₄H₃₀₄N₅₈O₅₈

Exact Mass: 4374.26 Da

Molecular Weight: 4376.91 g/mol

Example 22 was synthesized in a 15 μmol scale. The yield was 0.8 mg (1.2% of theory).

Example 22 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 50 min (Eluent A=0.1% TFA in water; Eluent B=0.08% TFA in ACN; flow rate=0.6 mL/min; λ=220 nm). Rt=25.9 min, purity ≧95%

In addition, a Jupiter® 5 μm C18 300 Å column (Phenomenex, 250 mm×4.6 mm, 5 μm, 300 Å) was used, applying a linear gradient of 10% to 60% eluent B in A over 50 min (Eluent A=0.1% TFA in water; Eluent B=0.08% TFA in ACN; flow rate=0.6 mL/min; λ=220 nm). Rt=25.8 min, purity ≧95%

The observed mass was in correspondence to the calculated mass ESI Ion-Trap: m/z=1095.0 [M+4H]⁴⁺, 876.3 [M+5H]⁵⁺, 730.5 [M+6H]⁶⁺, 626.3 [M+7H]⁷⁺, 548.1 [M+8H]⁸⁺; MALDI-TOF: m/z=4375.2 [M+H]⁺, 2188.1 [M+2H]²⁺, 1459.1 [M+3H]³⁺

### Example 26: [G¹⁴, (D¹⁶, Dpr²¹)_{lac}]ADM(14-52)

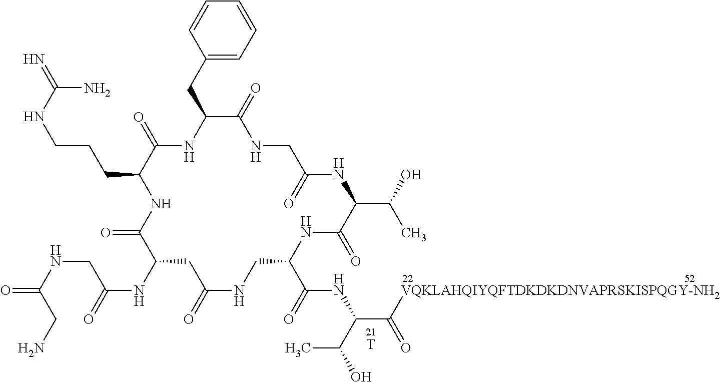

### ((3S,6S,12S,15S,18S)-18-(2-(2-aminoacetamido)acetamido)-12-benzyl-15-(3-guanidinopropyl)-6-((R)-1-hydroxyethyl)-5,8,11,14,17,20-hexaoxo-1,4,7,10,13,16-hexaazacycloicosane-3-carbonyl)-L-threonyl-ADM(22-52)

Chemical Formula: C₁₉₁H₂₉₈N₅₈O₅₈

Exact Mass: 4332.215 Da

Molecular Weight: 4334.83 g/mol

Example 26 was synthesized in a 15 μmol scale. The yield was 3.8 mg (5.8% of theory).

Example 26 was analyzed via analytical RP-HPLC using a Jupiter® 4 μm Proteo 90 Å column (Phenomenex, 250 mm×4.6 mm, 4 μm, 90 Å), applying a linear gradient of 10% to 60% eluent B in A over 50 min (Eluent A=0.1% TFA in water; Eluent B=0.08% TFA in ACN; flow rate=0.6 mL/min; λ=220 nm). Rt=26.1 min, purity ≧90%

In addition, a Jupiter® 5 μm C18 300 Å column (Phenomenex, 250 mm×4.6 mm, 5 μm, 300 Å) was used, applying a linear gradient of 10% to 60% eluent B in A over 50 min (Eluent A=0.1% TFA in water; Eluent B=0.08% TFA in ACN; flow rate=0.6 mL/min; λ=220 nm). Rt=26.1 min, purity ≧90%

The observed mass was in correspondence to the calculated mass ESI Ion-Trap: m/z=1084.5 [M+4H]⁴⁺, 867.9 [M+5H]⁵⁺, 723.5 [M+6H]⁶⁺, 620.3 [M+7H]⁷⁺, 542.9 [M+8H]⁸⁺; MALDI-TOF: m/z=4333.2 [M+H]⁺, 2167.1 [M+2H]²⁺.

### Palmitoylated Lactam-Bridged Adrenomedullin—Analogues 12, 13, and 18

### Synthesis:

Examples 12, 13, and 18 were synthesized using the general method described above.

The coupling sequences were as follows:

| | | | | AA of |
|---|---|---|---|---|
| Coupling | | | | human |
| Cycle | Example 12 | Example 13 | Example 18 | ADM |
| 1. | Tyr(tBu) | Tyr(tBu) | Tyr(tBu) | 52 |
| 2. | Gly | Gly | Gly | 51 |
| 3. | Gln(Trt) | Gln(Trt) | Gln(Trt) | 50 |
| 4. | Pro | Pro | Pro | 49 |
| 5. | Ser(tBu) | Ser(tBu) | Ser(tBu) | 48 |
| 6. | Ile | Ile | Ile | 47 |
| 7. | Lys(Boc) | Lys(Boc) | Lys(Boc) | 46 |
| 8. | Ser(tBu) | Ser(tBu) | Ser(tBu) | 45 |
| 9. | Arg(Pbf) | Arg(Pbf) | Arg(Pbf) | 44 |
| 10. | Pro | Pro | Pro | 43 |
| 11. | Ala | Ala | Ala | 42 |
| 12. | Val | Val | Val | 41 |
| 13. | Asn(Trt) | Asn(Trt) | Asn(Trt) | 40 |
| 14. | Asp(tBu) | Asp(tBu) | Asp(tBu) | 39 |
| 15. | Lys(Boc) | Lys(Boc) | Lys(Boc) | 38 |
| 16. | Asp(tBu) | Asp(tBu) | Asp(tBu) | 37 |
| 17. | Lys(Boc) | Lys(Boc) | Lys(Boc) | 36 |
| 18. | Asp(tBu) | Asp(tBu) | Asp(tBu) | 35 |
| 19. | Thr(tBu) | Thr(tBu) | Thr(tBu) | 34 |
| 20. | Phe | Phe | Phe | 33 |
| 21. | Gln(Trt) | Gln(Trt) | Gln(Trt) | 32 |
| 22. | Tyr(tBu) | Tyr(tBu) | Tyr(tBu) | 31 |
| 23. | Ile | Ile | Ile | 30 |
| 24. | Gln(Trt) | Gln(Trt) | Gln(Trt) | 29 |
| 25. | His(Trt) | His(Trt) | His(Trt) | 28 |
| 26. | Ala | Ala | Ala | 27 |
| 27. | Leu | Leu | Leu | 26 |
| 28. | Lys(Boc) | Lys(Boc) | Lys(Boc) | 25 |
| 29. | Gln(Trt) | Gln(Trt) | Gln(Trt) | 24 |
| 30. | Val | Val | Val | 23 |
| 31. | Thr(tBu) | Thr(tBu) | Thr(tBu) | 22 |
| 32. | Lys(Mmt) | Glu(OPp) | | 21 |
| 33. | Thr(tBu) | Thr(tBu) | | 20 |
| 34. | Gly | Gly | | 19 |
| 35. | Phe | Phe | | 18 |
| 36. | Arg(Pbf) | Arg(Pbf) | | 17 |
| 37. | Glu(OPp) | Lys(Mmt) | | 16 |
| 38. | Gly | Gly | | 15 |

After automated synthesis of the sequence ADM(15-52), for Examples 12 and 13 the N-terminal amino acid Boc-Lys(Fmoc)-OH was coupled manually with HOBt and DIC in 5-fold molar excess (75 μmol). The reaction was performed in DMF as solvent for 24 h.

For Example 18, Fmoc-Glu(OPp)-OH (AA 21) was coupled manually using a 5-fold molar excess of amino acid, HOBt and DIC in DMF as solvent for approx. 24 h. After Fmoc-deprotection, the peptide sequence was elongated automatically using the general method described above.

The coupling sequence was as follows:

| | | |
|---|---|---|
| 33. | Thr(tBu) | 20 |
| 34. | Gly | 19 |
| 35. | Phe | 18 |
| 36. | Arg(Pbf) | 17 |

For Example 18, Fmoc-Dpr(Mtt)-OH (AA 16) was afterwards coupled manually using a 5-fold molar excess of amino acid, HOBt and DIC in DMF as solvent for approx. 24 h.

Removal of the OPp, Mmt and Mtt protection groups was achieved by treatment of the resin (20×2 min) with a cleavage cocktail consisting of TFA/TIS/DCM (1:5:94, v/v/v) for Examples 12 and 13 and TFA/TIS/DCM (2:5:93, v/v/v) for Example 18. Subsequently, the resin was washed (2×5 min) with 5% DIPEA in DMF for Examples 12 and 13 and with 2% DIPEA in DMF for Example 16.

The lactam-bridge was introduced via formation of an amide bond between the side chains of AA 16 and AA 21. For Examples 12 and 13 the reaction was performed using a 10-fold excess (150 μmol) and for Example 18 a 6-fold excess (90 μmol) of HOBt and DIC in DMF as solvent for approx. 24 h at room temperature.

For Example 18 the amino acids Fmoc-Gly-OH (AA 15) and Boc-Lys(Fmoc)-OH (AA 14) were coupled manually with HOBt and DIC in 5-fold molar excess (75 μmol). The reactions were performed in DMF as solvent for 24 h.

Subsequently, Fmoc was removed from the N-terminal lysine using 30% piperidine in DMF (v/v) for twice 10 min.

Palmitoylation of the free lysine side chain was achieved using a 5-fold excess (75 μmol) of palmitic acid, HOBt and DIC in DMF as solvent for approx. 24 h.

Cleavage of the peptides from the resin and simultaneous side chain deprotection was achieved with TFA/TIS/H2O (90:5:5, v/v/v) for Examples 12 and 13 and TFA/TA/EDT (90:7:3, v/v/v) for Example 18 for 3 h. The peptides were precipitated and washed with ice-cold diethyl ether and subsequently lyophilized.

Purification of the crude peptides was performed using preparative RP-HPLC on a C18-column (Phenomenex Jupiter 10 u Proteo 90 Å: 250 mm×21.2 mm, 10 μm, 90 Å). For Example 12, a linear gradient of 10% to 60% eluent B in A over 40 min, for Example 13, a linear gradient of 20% to 70% eluent B in A over 50 min was applied (Eluent A=0.1% TFA in water; Eluent B=0.08% TFA in ACN). The flow rate was 10 mL/min for Example 12 and 15 mL/min for Example 13, UV detection was measured at λ=220 nm.

Purification of Example 18 was performed using preparative RP-HPLC on a C18 column (Kinetex® 5 μm XB-C18 100 Å: 250 mm×21.2 mm, 5 μm). A linear gradient of 20% to 70% eluent B in A over 50 min was applied (Eluent A=0.1% TFA in water; Eluent B=0.08% TFA in ACN). The flow rate was 20 mL/min, UV detection was measured at λ=220 nm.

### Analytics:

The identity of the peptides was confirmed via MALDI-MS (UltraflexIII, Bruker) and ESI-MS (HCT, Bruker). The purities were analyzed using analytical RP-HPLC.

### Example 12: [K¹⁴(PAM), (E¹⁶,K²¹)_{lac}]ADM(14-52)

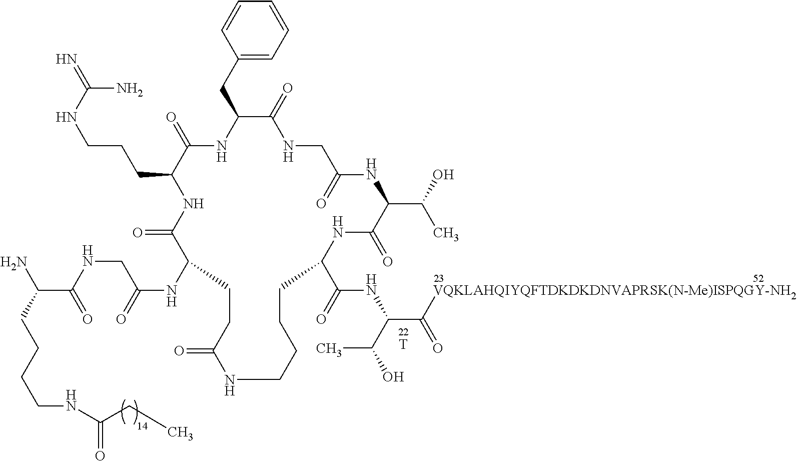

### ((2S,5S,11S,14S,23S)-23-(2-((S)-2-amino-6-palmitamidohexanamido)acetamido)-5-benzyl-2-(3-guanidinopropyl)-11-((R)-1-hydroxyethyl)-3,6,9,12,20,24-hexaoxo-1,4,7,10,13,19-hexaazacyclotetracosane-14-carbonyl)-L-threonyl-ADM(22-52)

Chemical Formula: C₂₁₅H₃₄₅N₅₉O₅₉

Exact Mass: 4697.58 Da

Molecular Weight: 4700.47 g/mol

Example 12 was synthesized in a 15 μmol scale. The yield was 5.3 mg (7.6% of theory).

Example 12 was analyzed via analytical RP-HPLC using a Jupiter® 5 μm C18 300 Å column (Phenomenex, 250 mm×4.6 mm, 5 μm, 300 Å) applying a linear gradient of 20% to 70% eluent B in A over 50 min. Rₜ=39.7 min, purity ≧95%.

In addition, a Jupiter® 4 μm Proteo 90 Å column (Phenomenex, 250 mm×4.6 mm, 4 μm, 90 Å) was used, applying a linear gradient of 10% to 100% eluent B in A over 60 min (Eluent A=0.1% TFA in water; Eluent B=0.08% TFA in ACN; flow rate=0.6 mL/min; λ=220 nm). Rₜ=30.7 min, purity ≧95%.

The observed mass was in correspondence to the calculated mass. ESI Ion-Trap: m/z=1175.9 [M+4H]⁴⁺, 941.0 [M+5H]⁵⁺, 784.4 [M+6H]⁶⁺, 672.6 [M+7H]⁷⁺, 588.8 [M+8H]⁸⁺; MALDI-TOF: m/z=4698.8 [M+H]⁺, 2349.7 [M+2H]²⁺.

### Example 13: [K¹⁴(PAM), (K¹⁶,E²¹)_{lac}]ADM(14-52)

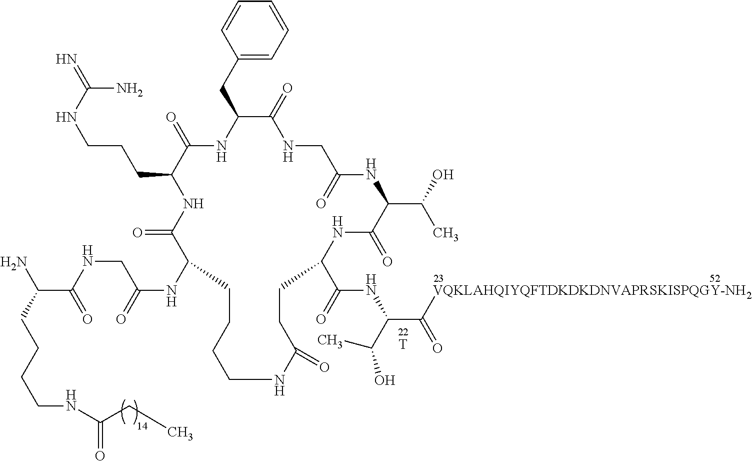

### ((2S,5S,11S,14S,23S)-23-(2-((S)-2-amino-6-palmitamidohexanamido)acetamido)-5-benzyl-2-(3-guanidinopropyl)-11-((R)-1-hydroxyethyl)-3,6,9,12,17,24-hexaoxo-1,4,7,10,13,18-hexaazacyclotetracosane-14-carbonyl)-L-threonyl-ADM(22-52)

Chemical Formula: C₂₁₅H₃₄₅N₅₉O₅₉

Molecular Weight: 4700.47 g/mol

Example 13 was synthesized in a 15 μmol scale. The yield was 3.5 mg (5.0% of theory).

Example 13 was analyzed via analytical RP-HPLC using a Jupiter® 5 μm C18 300 Å column (Phenomenex, 250 mm×4.6 mm, 5 μm, 300 Å) applying a linear gradient of 10% to 60% eluent B in A over 50 min.). Rₜ=41.2 min, purity ≧95%.

Also, a linear gradient of 10% to 100% eluent B in A over 60 min was used. (Eluent A=0.1% TFA in water; Eluent B=0.08% TFA in ACN; flow rate=0.6 mL/min; λ=220 nm). Rₜ=30.8 min, purity ≧95%.

The observed mass was in correspondence to the calculated mass. ESI Ion-Trap: m/z=1175.9[M+4H]⁴⁺, 941.0 [M+5H]⁵⁺, 784.4 [M+6H]⁶⁺, 672.6 [M+7H]⁷⁺, 588.8 [M+8H]⁸⁺; MALDI-TOF: m/z=4698.7 [M+H]⁺, 2349.75 [M+2H]²⁺.

### Example 18: [K¹⁴(PAM), (Dpr¹⁶,E²¹)_{lac}]ADM(14-52)

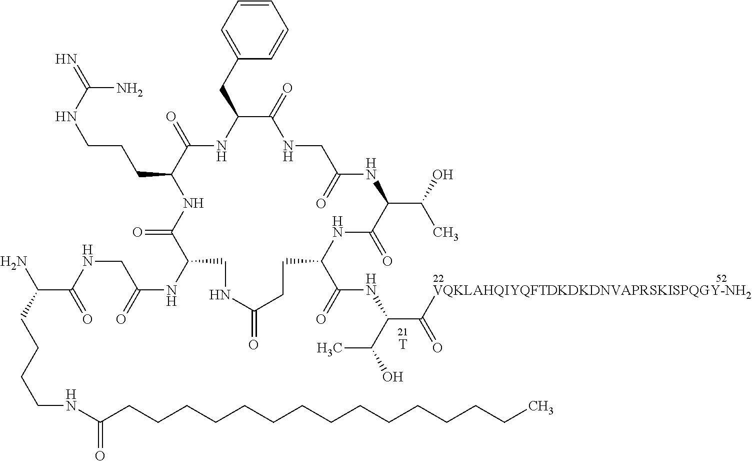

### ((3S,9S,12S,15S,21S)-15-(2-((S)-2-amino-6-palmitamidohexanamido)acetamido)-9-benzyl-12-(3-guanidinopropyl)-3-((R)-1-hydroxyethyl)-2,5,8,11,14,18-hexaoxo-1,4,7,10,13,17-hexaazacyclohenicosane-21-carbonyl)-L-threonyl-ADM(22-52)

Chemical Formula: C₂₁₂H₃₃₉N₅₉O₅₉

Exact Mass: 4655.53 Da

Molecular Weight: 4658.40 g/mol

Example 18 was synthesized in a 15 μmol scale. The yield was 2.4 mg (3.4% of theory).

Example 18 was analyzed via analytical RP-HPLC using a Jupiter® 5 μm C18 300 Å column (Phenomenex, 250 mm×4.6 mm, 5 μm, 300 Å) applying a linear gradient of 20% to 70% eluent B in A over 40 min.). Rₜ=34.1 min, purity ≧95%.

In addition, a Jupiter® 4 μm Proteo 90 Å column (Phenomenex, 250 mm×4.6 mm, 4 μm, 90 Å) was used, applying a linear gradient of 20% to 70% eluent B in A over 50 min (Eluent A=0.1% TFA in water; Eluent B=0.08% TFA in ACN; flow rate=0.6 mL/min; λ=220 nm). Rₜ=29.4 min, purity ≧95%.

The observed mass was in correspondence to the calculated mass. ESI Ion-Trap: m/z=1165.5 [M+4H]⁴⁺, 932.7 [M+5H]⁵⁺, 777.3 [M+6H]⁶⁺, 666.5 [M+7H]⁷⁺, 583.3 [M+8H]⁸⁺; MALDI-TOF: m/z=4656.6 [M+H]⁺, 2328.7 [M+2H]²⁺.

Adrenomedullin-Analogue 14 with N-methylated Isoleucin⁴⁷

### Synthesis:

The sequence ADM(48-52) was synthesized using the general method described above. The coupling sequence was as follows:

| Coupling Cycle | Example 14 | AA of human ADM |
|---|---|---|
| 1. | Tyr(tBu) | 52 |
| 2. | Gly | 51 |
| 3. | Gln(Trt) | 50 |
| 4. | Pro | 49 |
| 5. | Ser(tBu) | 48 |

After automated synthesis of the sequence ADM(48-52), Fmoc-protected N-methylated Isoleucin was coupled manually with HOBt and DIC in 5-fold molar excess. The reaction was performed in DMF as solvent for 24 h.

After removal of the Fmoc protecting group, Fmoc-Lys(Boc)-OH was coupled manually with a 5-fold molar excess of amino acid and a 10-fold molar (150 μmol) excess of HOBt and DIC in DMF/DCM/NMP (1:1:1, v/v/v). The reaction was performed at 50° C. whilst shaking with 1300 rpm (Thermomixer, Eppendorf) for 24 h.

Subsequently, elongation of the peptide chain was performed using the general method described above. The coupling sequence was as follows:

| Coupling Cycle | Example 14 | AA of human ADM |
|---|---|---|
| 8. | Ser(tBu) | 45 |
| 9. | Arg(Pbf) | 44 |
| 10. | Pro | 43 |
| 11. | Ala | 42 |
| 12. | Val | 41 |
| 13. | Asn(Trt) | 40 |
| 14. | Asp(tBu) | 39 |
| 15. | Lys(Boc) | 38 |
| 16. | Asp(tBu) | 37 |
| 17. | Lys(Boc) | 36 |
| 18. | Asp(tBu) | 35 |
| 19. | Thr(tBu) | 34 |
| 20. | Phe | 33 |
| 21. | Gln(Trt) | 32 |
| 22. | Tyr(tBu) | 31 |
| 23. | Ile | 30 |
| 24. | Gln(Trt) | 29 |
| 25. | His(Trt) | 28 |
| 26. | Ala | 27 |
| 27. | Leu | 26 |
| 28. | Lys(Boc) | 25 |
| 29. | Gln(Trt) | 24 |
| 30. | Val | 23 |
| 31. | Thr(tBu) | 22 |
| 32. | Lys(Mmt) | 21 |
| 33. | Thr(tBu) | 20 |
| 34. | Gly | 19 |
| 35. | Phe | 18 |
| 36. | Arg(Pbf) | 17 |
| 37. | Glu(OPp) | 16 |
| 38. | Gly | 15 |

After elongation the N-terminal amino acid Boc-Lys(Fmoc)-OH was coupled manually with HOBt and DIC in a 5-fold molar excess (75 μmol) in DMF as solvent for approx. 24 h.

Removal of the OPp and Mmt protection groups was achieved by treatment of the resin (15×2 min) with a cleavage cocktail consisting of TFA/TIS/DCM (2:5:93, v/v/v). Subsequently, the resin was washed (2×5 min) with 5% DIPEA in DMF.

The lactam-bridge was introduced via formation of an amide bond between the side chains of AA 16 and AA 21. The reaction was performed using a 10-fold excess (150 μmol) of HOBt and DIC in DMF as solvent for approx. 24 h.

Subsequently, Fmoc was removed from the N-terminal lysine using 30% piperidine in DMF (v/v) for twice 10 min.

Palmitoylation of the free lysine side chain was achieved using a 5-fold excess (75 μmol) of palmitic acid, HOBt and DIC in DMF as solvent for approx. 24 h.

Cleavage of the peptide from the resin and simultaneous side chain deprotection was achieved with TFA/TA/EDT (90:7:3, v/v/v) for approx. 3 h. The peptides were precipitated and washed with ice-cold diethyl ether and subsequently lyophilized.

Purification of the crude peptide was performed using preparative RP-HPLC on a C18-column (Phenomenex Jupiter 10 u Proteo 90 Å: 250 mm×21.2 mm, 10 μm, 90 Å). A linear gradient of 20% to 70% eluent B in A over 50 min was applied (Eluent A=0.1% TFA in water; Eluent B=0.08% TFA in ACN). The flow rate was 10 mL/min, UV detection was measured at λ=220 nm.

### Analytics:

The identity of the peptide was confirmed via MALDI-MS (UltraflexIII, Bruker) and ESI-MS (HCT, Bruker). The purities were analyzed using analytical RP-HPLC.

### Example 14: [K¹⁴(PAM), (E¹⁶,K²¹)_{lac}, N-Me-I⁴⁷]ADM(14-52)

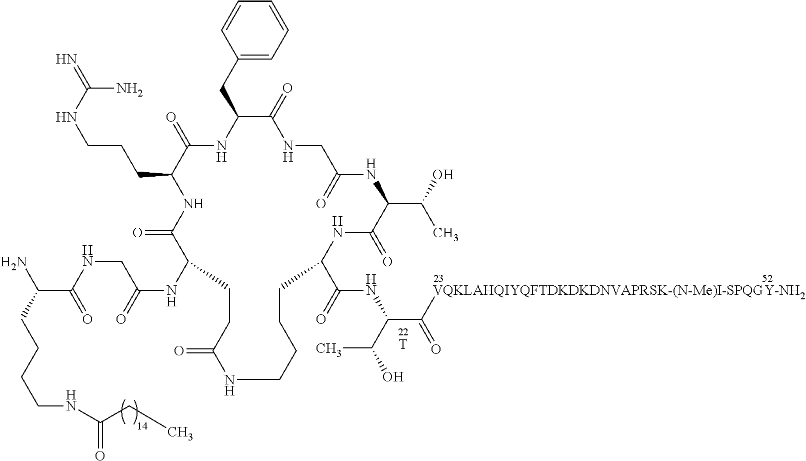

### ((2S,5S,11S,14S,23S)-23-(2-((S)-2-amino-6-palmitamidohexanamido)acetamido)-5-benzyl-2-(3-guanidinopropyl)-11-((R)-1-hydroxyethyl)-3,6,9,12,20,24-hexaoxo-1,4,7,10,13,19-hexaazacyclotetracosane-14-carbonyl)-L-threonyl-[N-Me-I⁴⁷]ADM(22-52)

Chemical Formula: C₂₁₆H₃₄₇N₅₉O₅₉

Exact Mass: 4711.60 Da

Molecular Weight: 4714.49 g/mol

Example 14 was synthesized in a 15 μmol scale. The yield was 0.6 mg (0.9% of theory).

Example 14 was analyzed via analytical RP-HPLC using a Jupiter® 5 μm C18 300 Å column (Phenomenex, 250 mm×4.6 mm, 5 μm, 300 Å) applying a linear gradient of 20% to 70% eluent B in A over 40 min (Eluent A=0.1% TFA in water; Eluent B=0.08% TFA in ACN; flow rate=0.6 mL/min; λ=220 nm). Rt=34.3 min, purity ≧90%.

In addition, a Vydac 218TP C18 column (Grace Vydac, 250 mm×4.6 mm, 5 μm, 300 Å) was used, applying a linear gradient of 20% to 70% eluent B in A over 40 min (Eluent A=0.1% TFA in water; Eluent B=0.08% TFA in ACN; flow rate=0.6 mL/min; λ=220 nm). Rt=30.9 min, purity ≧90%.

The observed mass was in correspondence to the calculated mass. ESI Ion-Trap: m/z=1179.6 [M+4H]⁴⁺, 943.8 [M+5H]⁵⁺, 786.8 [M+6H]⁶⁺, 674.5 [M+7H]⁷⁺, 590.3 [M+8H]⁸⁺; MALDI-TOF: m/z=4712.74 [M+H]⁺, 2356.7 [M+2H]²⁺.

Adrenomedullin-Analogue 27 with N-methylated Lysine⁴⁶

### Synthesis:

The sequence ADM(47-52) was synthesized using the general method described above. The coupling sequence was as follows:

| Coupling Cycle | Example 27 | AA of human ADM |
|---|---|---|
| 1. | Tyr(tBu) | 52 |
| 2. | Gly | 51 |
| 3. | Gln(Trt) | 50 |
| 4. | Pro | 49 |
| 5. | Ser(tBu) | 48 |
| 6. | Ile(tBu) | 47 |

After automated synthesis of the sequence ADM(47-52), Fmoc-protected N-methylated lysine was coupled manually with HOBt and DIC in 5-fold molar excess (75 μmol). The reaction was performed in DMF as solvent for 24 h.

After removal of the Fmoc protecting group, Fmoc-Ser(tBu)-OH was coupled manually with a 5-fold molar excess (75 μmol) of amino acid and a 10-fold molar excess (150 μmol) excess of HOBt and DIC in DMF/DCM/NMP (1:1:1, v/v/v). The reaction was performed at 50° C. in whilst shaking with 1300 rpm (Thermomixer, Eppendorf) for 24 h.

Subsequently, elongation of the peptide chain was performed using the general method described above. The coupling sequence was as follows:

| Coupling Cycle | Example 27 | AA of human ADM |
|---|---|---|
| 9. | Arg(Pbf) | 44 |
| 10. | Pro | 43 |
| 11. | Ala | 42 |
| 12. | Val | 41 |
| 13. | Asn(Trt) | 40 |
| 14. | Asp(tBu) | 39 |
| 15. | Lys(Boc) | 38 |
| 16. | Asp(tBu) | 37 |
| 17. | Lys(Boc) | 36 |
| 18. | Asp(tBu) | 35 |
| 19. | Thr(tBu) | 34 |
| 20. | Phe | 33 |
| 21. | Gln(Trt) | 32 |
| 22. | Tyr(tBu) | 31 |
| 23. | Ile | 30 |
| 24. | Gln(Trt) | 29 |
| 25. | His(Trt) | 28 |
| 26. | Ala | 27 |
| 27. | Leu | 26 |
| 28. | Lys(Boc) | 25 |
| 29. | Gln(Trt) | 24 |
| 30. | Val | 23 |
| 31. | Thr(tBu) | 22 |

Fmoc-Glu(OPp)-OH was coupled manually with HOBt and DIC in 5-fold molar excess (75 μmol). The reaction was performed in DMF as solvent for 24 h.

The peptide chain was elongated automatically using the general method described above. The coupling sequence was as follows:

| Coupling Cycle | Example 27 | AA of human ADM |
|---|---|---|
| 33. | Thr(tBu) | 20 |
| 34. | Gly | 19 |
| 35. | Phe | 18 |
| 36. | Arg(Pbf) | 17 |

Fmoc-Dpr(Mtt)-OH was coupled manually with HOBt and DIC in 5-fold molar excess (75 μmol). The reaction was performed in DMF as solvent for 24 h.

Removal of the OPp and Mtt protection groups was achieved by treatment of the resin (12×2 min) with a cleavage cocktail consisting of TFA/TIS/DCM (2:5:93, v/v/v). Subsequently, the resin was washed (2×5 min) with 2% DIPEA in DMF.

The lactam-bridge was introduced via formation of an amide bond between the side chains of AA 16 and AA 21. The reaction was performed using a 6-fold excess (90 μmol) of HOBt and DIC in DMF as solvent for approx. 24 h.

Subsequently, Fmoc-Gly-OH and Boc-Lys(Fmoc)-OH were coupled manually with HOBt and DIC in 5-fold molar excess (75 μmol) in DMF for approx. 24 h. Fmoc was removed with 30% piperidine in DMF (v/v) for twice 10 min prior to each coupling step and after finishing the synthesis to generate a free lysine side chain.

Palmitoylation of the free lysine side chain was achieved using a 5-fold excess (75 μmol) of palmitic acid, HOBt and DIC in DMF as solvent for approx. 24 h.

Cleavage of the peptide from the resin and simultaneous side chain deprotection was achieved with TFA/TA/EDT (90:7:3, v/v/v) for approx. 3 h. The peptides were precipitated and washed with ice-cold diethyl ether and subsequently lyophilized.

Purification of the crude peptide was performed using preparative RP-HPLC on a C18-column (XBridgeBEH130 Prep C18 10 m OBD: 250 mm×19 mm, 10 μm). A linear gradient of 10% to 70% eluent B in A over 60 min was applied (Eluent A=0.1% TFA in water; Eluent B=0.08% TFA in ACN). The flow rate was 20 mL/min, UV detection was measured at λ=220 nm.

### Analytics:

The identity of the peptide was confirmed via MALDI-MS (UltraflexIII, Bruker) and ESI-MS (HCT, Bruker). The purities were analyzed using analytical RP-HPLC.

### Example 27: [K¹⁴(PAM), (Dpr¹⁶,E²¹)_{lac}, N-Me-K⁴⁶]ADM(14-52)

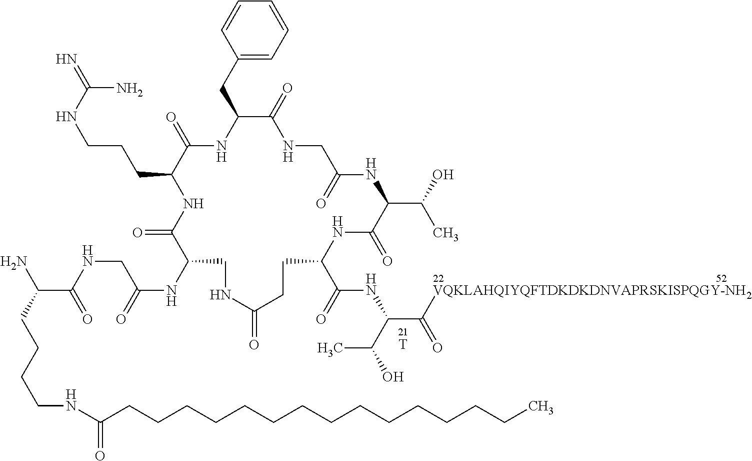

### ((3S,9S,12S,15S,21S)-15-(2-((S)-2-amino-6-palmitamidohexanamido)acetamido)-9-benzyl-12-(3-guanidinopropyl)-3-((R)-1-hydroxyethyl)-2,5,8,11,14,18-hexaoxo-1,4,7,10,13,17-hexaazacyclohenicosane-21-carbonyl)-L-threonyl-[N-Me-K⁴⁶]ADM(22-52)

Chemical Formula: C₂₁₃H₃₄₁N₅₉O₅₉

Exact Mass: 4669.55 Da

Molecular Weight: 4672.43 g/mol

Example 27 was synthesized in a 15 μmol scale. The yield was 0.6 mg (0.9% of theory).

Example 27 was analyzed via analytical RP-HPLC using a Jupiter® 5 μm C18 300 Å column (Phenomenex, 250 mm×4.6 mm, 5 μm, 300 Å) applying a linear gradient of 20% to 70% eluent B in A over 40 min (Eluent A=0.1% TFA in water; Eluent B=0.08% TFA in ACN; flow rate=0.6 mL/min; λ=220 nm). Rt=34.5 min, purity ≧95%.

In addition, a Jupiter® 4 μm Proteo 90 Å column (Phenomenex, 250 mm×4.6 mm, 4 μm, 90 Å) was used, applying a linear gradient of 10% to 100% eluent B in A over 60 min (Eluent A=0.1% TFA in water; Eluent B=0.08% TFA in ACN; flow rate=0.6 mL/min; λ=220 nm). Rₜ=29.6 min, purity ≧95%.

The observed mass was in correspondence to the calculated mass. ESI Ion-Trap: m/z=1168.7 [M+4H]⁴⁺, 935.5 [M+5H]⁵⁺, 779.6 [M+6H]⁶⁺, 668.5 [M+7H]⁷⁺, 584.9 [M+8H]⁸⁺; MALDI-TOF: m/z=4670.5 [M+H]⁺, 2336.6 [M+2H]²⁺.

Adrenomedullin—Analogues 15-17 with Disulfide Bond-Mimetics

### Synthesis:

The syntheses of compounds 15-17 were performed using automated peptide synthesis of the sequence ADM(22-52) as described in the general method. Subsequently, positions 21 to 14 were incorporated manually. The coupling sequence of ADM(22-52) was already shown for the lactam-bridged analogues 3-11 with non-proteinogenic amino acids.

The disulfide bond mimetics shown below were used as disulfide bond mimetics. They were Fmoc-protected at the N-terminus of the position replacing ADM-C²¹ and NAlloc, OAll-protected at the N- and C-termini of the position replacing ADM-C¹⁶.

The mimetics were coupled using a 5-fold molar excess of amino acid, HOBt and DIC in DMF as solvent for approx. 24 h. Fmoc-cleavage was performed using 20% piperidine in DMF (v/v) twice for 5 min.

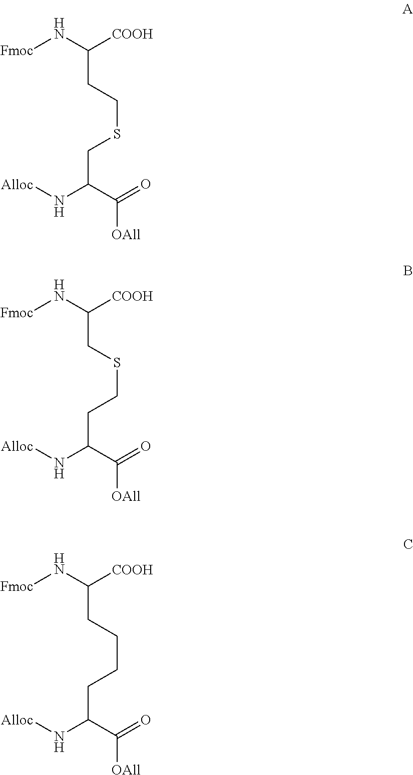

### A: Fmoc-[C¹⁶→U²¹](NAlloc, OAll)-OH; N-[(9H-fluoren-9-ylmethoxy)carbonyl]-S-[(2R)-3-oxo-3-(prop-2-en-1-yloxy)-2-{[(prop-2-en-1-yloxy)carbonyl]amino}propyl]-L-homocysteine

The compound was prepared according to the literature procedures P. J. Knerr, A. Tzekou, D. Ricklin, H. Qu, H. Chen, W. A. van der Donk, J. D. Lambris, ACS Chem. Biol. 2011, 6, 753-760 and H.-K. Cui, Y. Guo, Y. He, F.-L. Wang, H.-N. Chang, Y.-J. Wang, F.-M. Wu, C.-L. Tian, L. Liu, Angew. Chem. Int. Ed. 2013, 52, 9558-9562.

### B: Fmoc-[U¹⁶→U²¹](NAlloc, OAll)-OH; N-[(9H-fluoren-9-ylmethoxy)carbonyl]-3-{[(3S)-4-oxo-4-(prop-2-en-1-yloxy)-3-{[(prop-2-en-1-yloxy)carbonyl]amino}butyl]sulfanyl}-L-alanine

The compound was prepared according to the literature procedures P. J. Knerr, A. Tzekou, D. Ricklin, H. Qu, H. Chen, W. A. van der Donk, J. D. Lambris, ACS Chem. Biol. 2011, 6, 753-760 and H.-K. Cui, Y. Guo, Y. He, F.-L. Wang, H.-N. Chang, Y.-J. Wang, F.-M. Wu, C.-L. Tian, L. Liu, Angew. Chem. Int. Ed. 2013, 52, 9558-9562.

### C: Fmoc-[U¹⁶→U²¹](NAlloc, OAll)-OH; (2S,7S)-2-{[(9H-fluoren-9-ylmethoxy)carbonyl]amino}-8-oxo-8-(prop-2-en-1-yloxy)-7-{[(prop-2-en-1-yloxy)carbonyl]amino}octanoic acid

The compound was prepared according to the literature procedure H.-K. Cui, Y. Guo, Y. He, F.-L. Wang, H.-N. Chang, Y.-J. Wang, F.-M. Wu, C.-L. Tian, L. Liu, Angew. Chem. Int. Ed. 2013, 52, 9558-9562.

The following four amino acids of compounds 15-17 were coupled manually using a 5-fold molar excess of amino acid, HOBt and DIC in DMF as solvent for approx. 24 h. Fmoc-deprotection after the first three couplings was achieved by treatment of the resin with 20% piperidine in DMF (v/v) twice for 5 min.

The coupling sequence was as follows:

| Coupling Cycle | Examples 15-17 | AA of human ADM |
|---|---|---|
| 1. | Fmoc-Thr(tBu)-OH | 20 |
| 2. | Fmoc-Gly-OH | 19 |
| 3. | Fmoc-Phe-OH | 18 |
| 4. | Fmoc-Arg(Pbf)-OH | 17 |

Upon drying the resin at 40° C. under vacuum, Allyl- and Alloc protecting groups were removed using a 1.5-fold molar excess of TPP-Pd in CHCl₃/AcOH/NMM (37:2:1, v/v/v, 1.5 mL). The mixture was stirred under Argon atmosphere for 2 h. The resin was washed twice for 10 min each with 0.5% DIPEA in DMF and 0.5% DDTC in DMF (w/w). Fmoc-deprotection was achieved by treatment of the resin with 20% piperidine in DMF (v/v) twice for 5 min

The lactamization was performed using a 5-fold molar excess of HOBt and DIC using DMF as solvent for approx. 24 h.

The following two amino acids of compounds 15-17 were coupled manually using a 5-fold molar excess of amino acid, HOBt and DIC in DMF as solvent for approx. 24 h. Fmoc-deprotection after the first coupling was achieved by treatment of the resin with 20% piperidine in DMF (v/v) twice for 5 min.

The coupling sequence was as follows:

| Coupling Cycle | Examples 15-17 | AA of human ADM |
|---|---|---|
| 1. | Fmoc-Gly-OH | 15 |
| 2. | Boc-Gly-OH | 14 |

Cleavage of the peptides from the resin and simultaneous side chain deprotection was achieved with TFA/TA/EDT (90:7:3, v/v/v) for approx. 3 h. The peptides were precipitated and washed with ice-cold diethyl ether, and subsequently lyophilized.

Purification of the crude peptide was performed using preparative RP-HPLC on a C18-column (XBridge BEH130 Prep C18 10 μm OBD: 250 mm×19 mm, 10 μm). A linear gradient of 10% to 60% eluent B in A over 50 min was applied (Eluent A=0.1% TFA in water; Eluent B=0.08% TFA in ACN). The flow rate was 15 mL/min, UV detection was measured at λ=220 nm.

### Analytics:

The identity of the peptide was confirmed via MALDI-MS (UltraflexIII, Bruker) and ESI-MS (HCT, Bruker). The purities were analyzed using analytical RP-HPLC.

### Example 15: [G¹⁴, C¹⁶→U²¹]ADM(14-52)

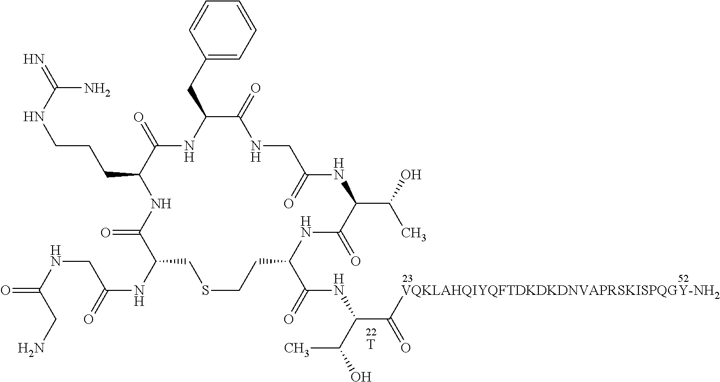

### ((4S,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1-thia-5,8,11,14,17-pentaazacycloicosane-4-carbonyl)-L-threonyl-ADM(22-52)

Chemical Formula: C₁₉₁H₂₉₉N₅₇O₅₇S

Exact Mass: 4335.197 Da

Molecular Weight: 4337.90 g/mol

Example 15 was synthesized in a 15 μmol scale. The yield was 1.6 mg (2.5% of theory).

Example 15 was analyzed via analytical RP-HPLC using Jupiter® 4 μm Proteo 90 Å column (Phenomenex, 250 mm×4.6 mm, 4 μm, 90 Å) applying a linear gradient of 10% to 60% eluent B in A over 40 min. Rₜ=25.3 min, purity ≧90%.

In addition, a Jupiter® 5 μm C18 300 Å column (Phenomenex, 250 mm×4.6 mm, 5 μm, 300 Å) applying a linear gradient of 10% to 60% eluent B in A over 40 min (Eluent A=0.1% TFA in water; Eluent B=0.08% TFA in ACN; flow rate=0.6 mL/min; λ=220 nm). Rₜ=26.0 min, purity ≧90%.

The observed mass was in correspondence to the calculated mass. ESI Ion-Trap: m/z=1085.4 [M+4H]⁴⁺, 868.5 [M+5H]⁵⁺, 723.9 [M+6H]⁶⁺, 602.7 [M+7H]⁷⁺, 543.3 [M+8H]⁸⁺; MALDI-TOF: m/z=4336.2 [M+H]⁺, 2168.6 [M+2H]²⁺.

### Example 16: [G¹⁴, U¹⁶→C²¹]ADM(14-52)

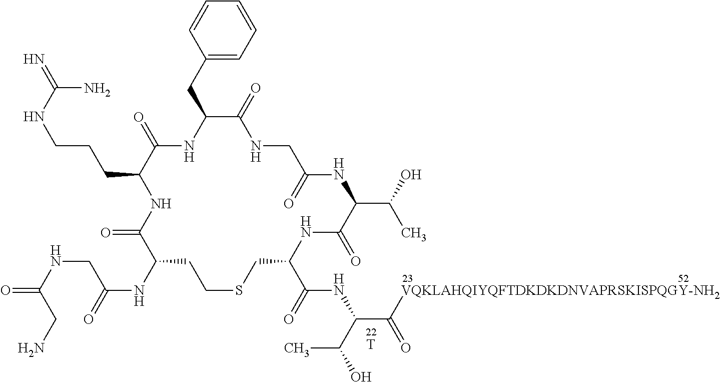

### ((3R,6S,12S,15S,18S)-18-(2-(2-aminoacetamido)acetamido)-12-benzyl-15-(3-guanidinopropyl)-6-((R)-1-hydroxyethyl)-5,8,11,14,17-pentaoxo-1-thia-4,7,10,13,16-pentaazacycloicosane-3-carbonyl)-L-threonyl-ADM(22-52)

Chemical Formula: C₁₉₁H₂₉₉N₅₇O₅₇S

Exact Mass: 4335.197 Da

Molecular Weight: 4337.90 g/mol

Example 16 was synthesized in a 15 μmol scale. The yield was 2.2 mg (3.4% of theory).

Example 16 was analyzed via analytical RP-HPLC using Phenomenex Jupiter 4 u Proteo 90 Å (Phenomenex, 250 mm×4.6 mm, 4 μm, 90 Å) applying a linear gradient of 10% to 60% eluent B in A over 40 min and a flow rate of 0.6 mL/min. Rₜ=25.2 min.

In addition, a Phenomenex Jupiter 5 u Proteo 300 Å column was used (Phenomenex, 250 mm×4.6 mm, 5 μm, 300 Å) applying a linear gradient of 10% to 60% eluent B in A over 40 min and a flow rate of 0.6 mL/min. Rₜ=29.8 min.

The observed mass was in correspondence to the calculated mass. ESI Ion-Trap: m/z=1085.6 [M+4H]⁴⁺, 868.5 [M+5H]⁵⁺, 723.9 [M+6H]⁶⁺, 602.7 [M+7H]⁷⁺, 543.2 [M+8H]⁸⁺; MALDI-TOF: m/z=4336.3 [M+H]⁺, 2168.6 [M+2H]²⁺.

### Example 17: [G¹⁴, U¹⁶→U²¹]ADM(14-52)

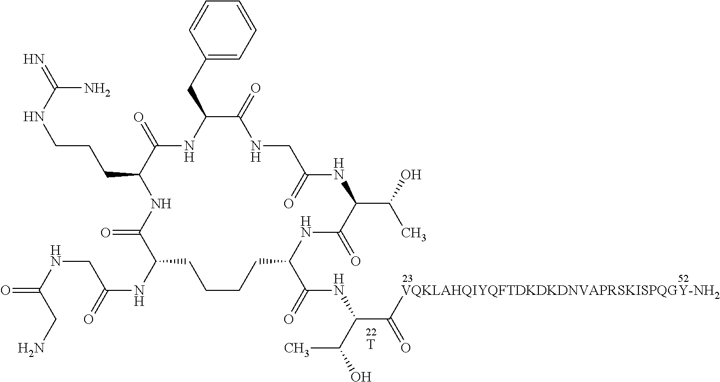

### ((2S,5S,11S,14S,19S)-19-(2-(2-aminoacetamido)acetamido)-5-benzyl-2-(3-guanidinopropyl)-11-((R)-1-hydroxyethyl)-3,6,9,12,20-pentaoxo-1,4,7,10,13-pentaazacycloicosane-14-carbonyl)-L threonyl-ADM(22-52)

Chemical Formula: C₁₉₂H₃₀₁N₅₇O₅₇

Exact Mass: 4317.241 Da

Molecular Weight: 4319.86 g/mol

Example 17 was synthesized in a 15 μmol scale. The yield was 2.6 mg (4.0% of theory).

Example 17 was analyzed via analytical RP-HPLC using Jupiter® 4 μm Proteo 90 Å column (Phenomenex, 250 mm×4.6 mm, 4 μm, 90 Å) applying a linear gradient of 10% to 60% eluent B in A over 40 min. Rₜ=23.2 min, purity ≧95%.

In addition, a Jupiter® 5 μm C18 300 Å column (Phenomenex, 250 mm×4.6 mm, 5 μm, 300 Å) applying a linear gradient of 10% to 60% eluent B in A over 40 min (Eluent A=0.1% TFA in water; Eluent B=0.08% TFA in ACN; flow rate=0.6 mL/min; λ=220 nm). Rₜ=23.4 min, purity ≧95%.

The observed mass was in correspondence to the calculated mass. ESI Ion-Trap: m/z=1080.9 [M+4H]⁴⁺, 864.9 [M+5H]⁵⁺, 721.0 [M+6H]⁶⁺, 618.2 [M+7H]⁷⁺, 540.9 [M+8H]⁸⁺; MALDI-TOF: m/z=4318.4 [M+H]⁺, 2159.7 [M+2H]²⁺.

Palmitoylated Adrenomedullin—Analogues 23-25 with Disulfide Bond-Mimetics

### Synthesis:

The synthesis of Examples 23-25 was performed using automated peptide synthesis of the sequence ADM(22-52) as described in the general method. Subsequently, positions 21 to 14 and the palmitoylation were incorporated manually. The coupling sequence of ADM(22-52) was already shown for the lactam-bridged analogues 3-11, 19-22, and 26.

Compounds A, B and C (shown above) were used as disulfide bond mimetics. They were Fmoc-protected at the N-terminus of the position replacing ADM-C²¹ and NAlloc, OAll-protected at the N- and C-termini of the position replacing ADM-C¹⁶.

The mimetics were coupled using a 5-fold molar excess of amino acid, HOBt and DIC in DMF as solvent for approx. 24 h. Fmoc-cleavage was performed using 20% piperidine in DMF (v/v) twice for 5 min.

Subsequently, elongation of amino acids 17-20 was performed using the general method described above. The coupling sequence was as follows:

| Coupling Cycle | Examples 23-25 | AA of human ADM |
|---|---|---|
| 1. | Fmoc-Thr(tBu)-OH | 20 |
| 2. | Fmoc-Gly-OH | 19 |
| 3. | Fmoc-Phe-OH | 18 |
| 4. | Fmoc-Arg(Pbf)-OH | 17 |

Upon drying the resin at 40° C. under vacuum, Allyl- and Alloc protecting groups were removed using a 1.5-fold molar excess of TPP-Pd in CHCl₃/AcOH/NMM (37:2:1, v/v/v, 1.5 mL). The mixture was stirred under Argon atmosphere for 2 h. The resin was washed twice for 10 min each with 0.5% DIPEA in DMF (v/v), 0.5% DDTC in DMF (w/w). Fmoc-deprotection was achieved by treatment of the resin with 30% piperidine in DMF (v/v) twice for 10 min.

The lactamization was performed using a 15-fold molar excess of HOBt and a 10-fold molar excess of DIC in DMF as solvent for 6-8 h.

The following two amino acids of Examples 23-25 were coupled manually using a 5-fold molar excess of amino acid, HOBt and DIC in DMF as solvent for approx. 24 h. Fmoc-deprotection after the first coupling was achieved by treatment of the resin with 30% piperidine in DMF (v/v) twice for 10 min.

The coupling sequence was as follows:

| | | | | AA of |
|---|---|---|---|---|
| Coupling | | | | human |
| Cycle | Example 23 | Example 24 | Example 25 | ADM |
| 1. | Fmoc-Gly-OH | | | 15 |
| 2. | Fmoc-Gly-OH | Fmoc-Lys(Boc)- | Boc-Lys(Fmoc)- | 14 |
| | | OH | OH | |

Subsequently, Fmoc was removed from the N-terminal amino acid using 30% piperidine in DMF (v/v) for twice 10 min.

Palmitoylation of the N-terminus (Examples 23 and 24) or the free lysine side chain (Examples 25) was achieved using a 5-fold excess (75 μmol) of palmitic acid, HOBt and DIC in DMF as solvent for approx. 24 h.

Cleavage of the peptides from the resin and simultaneous side chain deprotection was achieved with TFA/TA/EDT (90:7:3, v/v/v) for approx. 3 h. The peptides were precipitated and washed with ice-cold diethyl ether and subsequently lyophilized.

Purification of the crude peptide was performed using preparative RP-HPLC on a C18-column (Kinetex® 5 μm XB-C18 100 Å: 250 mm×21.2 mm, 5 μm). A linear gradient of 20% to 60% eluent B in A over 60 min was applied (Eluent A=0.1% TFA in water; Eluent B=0.08% TFA in ACN). The flow rate was 20 mL/min, UV detection was measured at λ=220 nm.

### Analytics:

The identity of the peptide was confirmed via MALDI-MS (UltraflexIII, Bruker) and ESI-MS (HCT, Bruker). The purities were analyzed using analytical RP-HPLC.

### Example 23: PAM[G¹⁴, C¹⁶→U²¹]ADM(14-52)

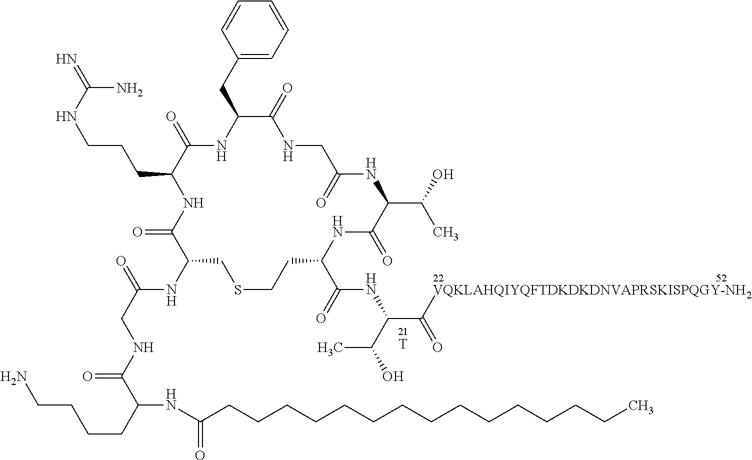

### ((4S,7S,13S,16S,19R)-13-benzyl-16-(3-guanidinopropyl)-7-(hydroxymethyl)-6,9,12,15,18-pentaoxo-19-(2-(2-palmitamidoacetamido)acetamido)-1-thia-5,8,11,14,17-pentaazacycloicosane-4-carbonyl)-L-threonyl-ADM(22-52)

Chemical Formula: C₂₀₇H₃₂₉N₅₇O₅₈S

Exact Mass: 4573.43 Da

Molecular Weight: 4576.31 g/mol

Example 23 was synthesized in a 15 μmol scale. The yield was 2.0 mg (2.9% of theory).

Example 23 was analyzed via analytical RP-HPLC using Jupiter® 4 μm Proteo 90 Å column (Phenomenex, 250 mm×4.6 mm, 4 μm, 90 Å) applying a linear gradient of 10% to 60% eluent B in A over 40 min(Eluent A=0.1% TFA in water; Eluent B=0.08% TFA in ACN; flow rate=0.6 mL/min; λ=220 nm). Rₜ=34.1 min, purity ≧95%.

In addition, a Jupiter® 5 μm C18 300 Å column (Phenomenex, 250 mm×4.6 mm, 5 μm, 300 Å) applying a linear gradient of 10% to 60% eluent B in A over 40 min (Eluent A=0.1% TFA in water; Eluent B=0.08% TFA in ACN; flow rate=0.6 mL/min; λ=220 nm). Rₜ=34.1 min, purity ≧95%.

The observed mass was in correspondence to the calculated mass. ESI Ion-Trap: m/z=916.4 [M+5H]⁵⁺, 763.7 [M+6H]⁶⁺, 654.8 [M+7H]⁷⁺; MALDI-TOF: m/z=4574.9 [M+H]⁺, 2287.1 [M+2H]²⁺.

### Example 24: PAM[K¹⁴, C¹⁶→U²¹]ADM(14-52)

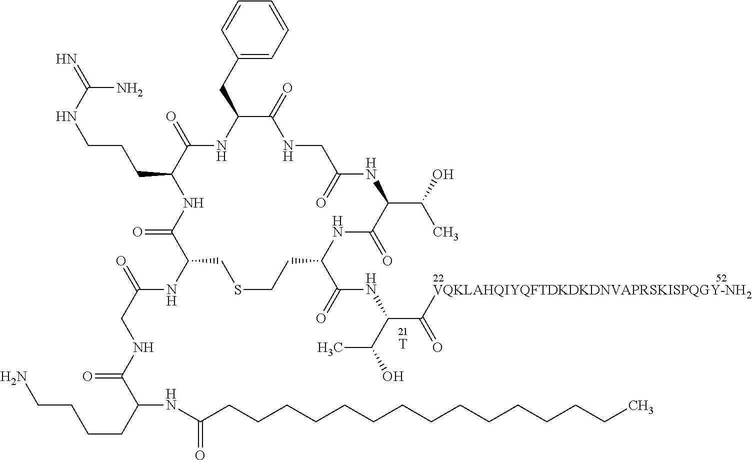

### ((4S,7S,13S,16S,19R)-19-(2-(6-amino-2-palmitamidohexanamido)acetamido)-13-benzyl-16-(3-guanidinopropyl)-7-((R)-1-hydroxyethyl)-6,9,12,15,18-pentaoxo-1-thia-5,8,11,14,17-pentaazacycloicosane-4-carbonyl)-L-threonyl-ADM(22-52)

Chemical Formula: C₂₁₁H₃₃₈N₅₈O₅₈S

Exact Mass: 4644.50 Da

Molecular Weight: 4647.43 g/mol

Example 24 was synthesized in a 15 μmol scale. The yield was 1.3 mg (1.9% of theory).

Example 24 was analyzed via analytical RP-HPLC using Jupiter® 4 μm Proteo 90 Å column (Phenomenex, 250 mm×4.6 mm, 4 μm, 90 Å) applying a linear gradient of 20% to 70% eluent B in A over 40 min(Eluent A=0.1% TFA in water; Eluent B=0.08% TFA in ACN; flow rate=0.6 mL/min; λ=220 nm). Rₜ=23.7 min, purity ≧95%.

In addition, a Jupiter® 5 μm C18 300 Å column (Phenomenex, 250 mm×4.6 mm, 5 μm, 300 Å) applying a linear gradient of 20% to 70% eluent B in A over 40 min (Eluent A=0.1% TFA in water; Eluent B=0.08% TFA in ACN; flow rate=0.6 mL/min; λ=220 nm). Rₜ=31.0 min, purity ≧95%.

The observed mass was in correspondence to the calculated mass. ESI Ion-Trap: m/z=1163.0 [M+4H]⁴⁺, 930.3 [M+5H]⁵⁺, 775.5 [M+6H]⁶⁺, 664.8 [M+7H]⁷⁺, 581.9 [M+8H]⁸⁺; MALDI-TOF: m/z=4645.5 [M+H]⁺, 2323.1 [M+2H]²⁺.

### Example 25: [K¹⁴(PAM), C¹⁶→U²¹]ADM(14-52)

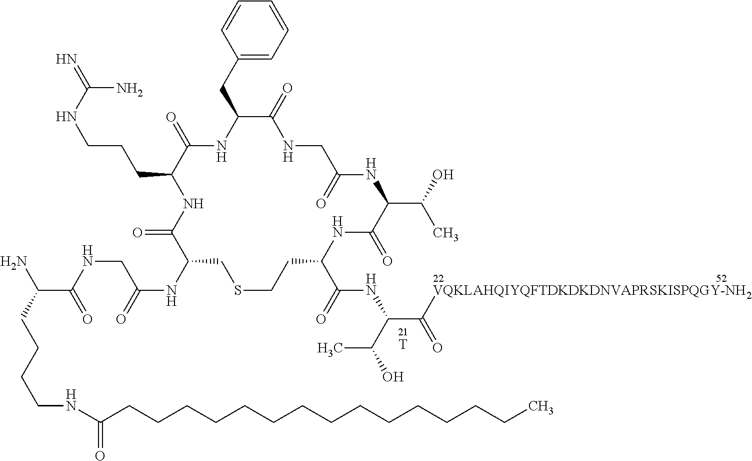

### ((4S,7S,13S,16S,19R)-19-(2-((S)-2-amino-6-palmitamidohexanamido)acetamido)-13-benzyl-16-(3-guanidinopropyl)-7-((R)-1-hydroxyethyl)-6,9,12,15,18-pentaoxo-1-thia-5,8,11,14,17-pentaazacycloicosane-4-carbonyl)-L-threonyl-ADM(22-52)

Chemical Formula: C₂₁₁H₃₃₈N₅₈O₅₈S

Exact Mass: 4644.50 Da

Molecular Weight: 4647.43 g/mol

Example 25 was synthesized in a 15 μmol scale. The yield was 2.0 mg (2.9% of theory).

Example 25 was analyzed via analytical RP-HPLC using Jupiter® 4 μm Proteo 90 Å column (Phenomenex, 250 mm×4.6 mm, 4 μm, 90 Å) applying a linear gradient of 20% to 70% eluent B in A over 40 min (Eluent A=0.1% TFA in water; Eluent B=0.08% TFA in ACN; flow rate=0.6 mL/min; λ=220 nm). Rₜ=24.4 min, purity ≧95%.

In addition, a Jupiter® 5 μm C18 300 Å column (Phenomenex, 250 mm×4.6 mm, 5 μm, 300 Å) applying a linear gradient of 20% to 70% eluent B in A over 40 min (Eluent A=0.1% TFA in water; Eluent B=0.08% TFA in ACN; flow rate=0.6 mL/min; λ=220 nm). Rₜ=33.5 min, purity ≧95%.

The observed mass was in correspondence to the calculated mass. ESI Ion-Trap: m/z=1162.7 [M+4H]⁴⁺, 930.4 [M+5H]⁵⁺, 775.6 [M+6H]⁶⁺, 664.9 [M+7H]⁷⁺, 581.9 [M+8H]⁸⁺; MALDI-TOF: m/z=4645.5 [M+H]⁺, 2323.1 [M+2H]²⁺.

### B. ASSESSMENT OF PHARMACOLOGICAL ACTIVITY

The following abbreviations are used:

| | |
|---|---|
| ACN | acetonitrile |
| BALF | bronchoalveolar lavage fluid |
| BP | arterial blood pressure |
| CHO | Chinese hamster ovary cells |
| CO | cardiac output |
| EC₅₀ | half-maximal effective concentration |
| EVWLI | extravascular lung water index |
| FiO₂ | fraction of inspired oxygen |
| FITC | Fluorescein isothiocyanate |
| HEPES | hydroxyethyl-piperazineethanesulfonic acid |
| HR | arterial heart rate |
| HUVEC | human umbilical venous cells |
| IBMX | 3-Isobutyl-1-methylxanthine |
| i.v. | intravenously |
| LPS | Lipopolysaccharide |
| LVP | left ventricular pressure |
| OA | oleic acid |
| PaO₂ | partial pressure of oxygen in arterial blood |
| PAP | pulmonary arterial pressure |
| PEG | Polyethylenglycol |
| s.c. | subcutaneously |
| TAM | 6-carboxytetramethylrhodamine |
| TEER | transendothelial electrical resistance |
| TFA | trifluoroacetic acid |
| TNF | Tumor Necrosis Factor |
| v/v | volume/volume |

The suitability of the compounds according to the invention for treatment of diseases can be demonstrated using the following assay systems:

### 1) Test Descriptions (In Vitro)

### 1a) Tests on a Recombinant Adrenomedullin-Receptor Reporter Cell

The activity of the compounds according to the invention was quantified with the aid of a recombinant Chinese hamster ovary (CHO) cell line that carries the human adrenomedullin-receptor. Activation of the receptor by ligands was measured by aequorin luminescence. Construction of the cell line and measurement procedure has been described in detail [Wunder F., Rebmann A., Geerts A, and Kalthof B., Mol Pharmacol, 73, 1235-1243 (2008)]. In brief: Cells were seeded on opaque 384-well microtiter plates at a density of 4000 cells/well and were grown for 24 h. After removal of culture medium, cells were loaded for 3 h with 0.6 μg/ml coelenterazine in Ca²⁺-free Tyrode solution (130 mM sodium chloride, 5 mM potassium chloride, 20 mM HEPES (4-(2-hydroxyethyl)-1-piperazineethanesulfonic acid), 1 mM magnesium chloride, and 4.8 mM sodium hydrogen carbonate, pH 7.4) supplemented with 0.2 mM 3-Isobutyl-1-methylxanthine (IBMX) in a cell culture incubator. Examples were added for 6 min in calcium²⁺-free Tyrode solution containing 0.1% bovine serum albumin. Immediately before adding calcium²⁺ to a final concentration of 3 mM measurement of the aequorin luminescence was started by use of a suitable luminometer. Luminescence was measured for 60 s. In a typical experiment compounds were tested in a concentration range of 1×10⁻¹³ to 3×10⁻⁶ M.

Representative EC₅₀ values for the embodiment examples are given in the following Table 1:

TABLE 1

| Example No. | EC₅₀ [nM] |
|---|---|
| 1 | 20.5 |
| 2 | >1000 |
| 3 | 49.6 |
| 4 | 21.1 |
| 5 | >1000 |
| 6 | >1000 |
| 7 | 5.26 |
| 8 | 17.3 |
| 9 | >1000 |
| 10 | 19.6 |
| 11 | 34.0 |
| 12 | 0.81 |
| 13 | >1000 |
| 14 | 32.0 |
| 15 | 0.07 |
| 16 | 1.54 |
| 17 | >1000 |
| 18 | 22 |
| 19 | >1000 |
| 20 | 27 |
| 21 | >1000 |
| 22 | 15 |
| 23 | 15 |
| 24 | 11 |
| 25 | 24 |
| 26 | 32 |
| 27 | 56 |
| wt ADM | 1.64 |

The EC₅₀ of wt ADM is mostly in the range of 0.5 nM to 2.5 nM.

### 1b) Transcellular Electrical Resistance Assays in Endothelial Cells

The activity of the compounds according to the invention is characterized in in vitro-permeability assays in human umbilical venous cells (HUVEC, Lonza). By use of the ECIS apparatus (ECIS: Electric Cell-substrate Impedance Sensing; Applied Biophysics Inc; Troy, N.Y.) changes of transendothelial electrical resistance (TEER) over an endothelial monolayer are continuously measured by use of a small gold electrode on which the cells have been seeded. HUVEC are grown on the 96-well sensor electrode plates (96W1E, Ibidi GmbH, Martinsried) to confluent monolayers and hyperpermeability can be induced by inflammatory stimuli such as Thrombin, TNF-α, IL-1β, VEGF, Histamine and hydrogen peroxide which all have been demonstrated to cause break down of endothelial cell contacts and reduction of TEER. Thrombin is used at a final concentration of 0.5 U/ml. Test compounds are added before or after addition of thrombin. In a typical experiment compounds are tested in a concentration range of 1×10⁻¹⁰ to 1×10⁻⁶ M.

Substances according to the present invention prevent break down of electrical resistance of a HUVEC monolayer after stimulation with thrombin dose dependently at concentrations of >1 nmol/L [FIG. 1].

### 1c) In Vitro-Permeability Assays in Endothelial Cells

In another in vitro model of endothelial hyperpermeability the activity of compounds according to the invention is examined with respect to modulation of macromolecular permeability. Human umbilical vein endothelial cells (HUVECS) are grown to confluency on fibronectin-coated Transwell® filter membranes (24-well plates, 6.5 mm-inserts with 0.4 μM polycarbonate membrane; Costar #3413) which separate an upper from a lower tissue culture chamber with endothelial cells growing on the bottom of the upper chamber. The medium of the upper chamber is supplemented with 250 μg/ml of 40 kDa FITC-Dextran (Invitrogen, D1844). Hyperpermeability of the monolayer is induced by addition of thrombin to a final concentration of 0.5 U/ml. Medium samples are collected from the lower chamber every 30 min and relative fluorescence as a parameter for changes of macromolecular permeability over time is measured in a suitable fluorimeter. Thrombin challenge induces almost a doubling of FITC-dextran transition across the endothelial monolayers. In a typical experiment compounds are tested in a concentration range of 1×10⁻¹⁰ to 1×10⁻⁶ M.

Substances according to the present invention reduce permeability of a HUVEC monolayer for 40 kDa FITC-Dextran after stimulation with thrombin dose dependently at concentrations of ≧0.3 nmol/L [FIG. 2].

### 1d) cAMP Assay

### Abbreviations

| | |
|---|---|
| CFP | cyan fluorescent protein |
| CLR | calcitonin receptor-like receptor |
| CRE | cAMP response element |
| DMEM | Dulbecco's Modified Eagle's Medium |
| DPBS | Dulbecco's Phosphate-Buffered Saline |
| ECFP | enhanced cyan fluorescent protein |
| EYFP | enhanced yellow fluorescent protein |
| FCS | fetal calf serum |
| RAMP2 | receptor activity-modifying protein 2 |

### Suppliers

| | |
|---|---|
| DMEM | Lonza |
| DPBS | Lonza |
| FCS | Biochrom |
| Ham's F-12 | Fluka |
| Metafectene^{R} Pro | Biontex |
| ONE-Glo ™ Luciferase Assay System | Promega |
| Poly-D-Lysin-hydrobromid stock solution | Sigma-Aldrich |
| 0.1% in H₂O | |

### Cell Culture

HEK-293 cells (human embryonic kidney cells) were cultured in Ham's F-12/DMEM (1/1; v/v) containing 15% FCS under humidified atmosphere at 37° C. and 5% CO₂ in 75 cm² cell culture flasks.

### Transient Co-Transfection of HEK293 Cells

Cells were cultured in 75 cm² flasks to 70-80% confluency. 45 μl Metafectene® Pro was diluted in 900 μl Ham's F-12/DMEM (1/1; v/v) and incubated for 20 min at room temperature. 9000 ng plasmid containing DNA of CLR fused to EYFP and 3000 ng plasmid containing DNA of RAMP2 fused to ECFP were dissolved in 900 μl Ham's F-12/DMEM (1/1; v/v). The plasmid solution was mixed with the Metafectene® Pro solution and incubated for 25 min at room temperature. Medium was removed from the cells and replaced by 6 ml Ham's F-12/DMEM (1/1; v/v) containing 15% FCS. After addition of transfection solution the cells were incubated for 3 h under humidified atmosphere at 37° C. and 5% CO₂.

For the second transfection 45 μl Metafectene® Pro was diluted in 900 μl Ham's F-12/DMEM (1/1; v/v) and incubated for 20 min at room temperature. 12000 ng of pGL4.29[luc2P/CRE/Hygro] plasmid containing DNA for the luciferase reporter gene luc2P (with CRE promotor region) were dissolved in 900 μl Ham's F-12/DMEM (1/1; v/v). The plasmid solution was mixed with the Metafectene® Pro solution and incubated for 25 min at room temperature. Medium was removed from the cells and replaced by 6 ml Ham's F-12/DMEM (1/1; v/v) containing 15% FCS. After addition of transfection solution the cells were incubated under humidified atmosphere at 37° C. and 5% CO₂ over night.

cAMP-Assay

Seeding of Transiently Transfected Cells in 96-Well-Plates

For the coating of 96-well-plates 50 μl of a Poly-D-Lysine solution (1 ml stock solution of Poly-D-Lysin-hydrobromid/10 ml DPBS) were pipetted in each well and incubated for 40 min. After removal of the Poly-D-Lysine each well was washed with 50 μl DPBS. Transiently transfected cells were detached from the cell culture flask by removal of the medium, twofold washing with 5 m1 DPBS and resuspending in 13 ml Ham's F-12/DMEM (1/1; v/v) containing 15% FCS. 90000 to 120000 cells in 150 μl Ham's F-12/DMEM (1/1; v/v) containing 15% FCS were seeded per well and the plates were incubated under humidified atmosphere at 37° C. and 5% CO₂ over night.

Cell Stimulation

For each ligand a serial dilution giving eight different concentrations was prepared using Ham's F-12/DMEM (1/1; v/v). Before stimulation the medium on the cells was replaced by 100 μl Ham's F-12/DMEM (1/1; v/v) and the plates were incubated for 1 h under humidified atmosphere at 37° C. and 5% CO₂. For stimulation the medium was removed again and the cells were incubated for 3 h in 80 μl of ligand-solution under humidified atmosphere at 37° C. and 5% CO₂. In addition 80 μl of a 5 μM forskolin solution (in Ham's F-12/DMEM (1/1; v/v)) was used as a positive control and 80 μl of Ham's F-12/DMEM (1/1; v/v) as a negative control. Each concentration and the controls were tested as triplicates.

Luminescence Measurement

After 3 h of stimulation, the solutions were removed and the cells were washed with 50 μl of Ham's F-12/DMEM (1/1; v/v) per well. After 10 min incubation in 30 μl of Ham's F-12/DMEM (1/1; v/v) at room temperature, 30 μl of luciferase-solution (ONE-Glo™ Luciferase Assay System) was added and the luminescence was directly measured using an Infinite M200 (Tecan).

Data Analysis

Data analysis of the luminescence measurement was carried out with GraphPad Prism 5. Therefore the measured luminescence values of each plate were first corrected on the base of the respective average of forskolin stimulation. Afterwards they were normalized to [G¹⁴]ADM(14-52) which was used as standard peptide in every assay. After correction and normalization data was analysed using non-linear regression giving dose-response curves for each tested ligand.

TABLE 2
Results cAMP assay

| Example No. | EC₅₀ [nM] |
|---|---|
| 1 | 31 |
| 2 | >1000 |
| 3 | 67 |
| 4 | 21 |
| 5 | 112 |
| 6 | 303 |
| 7 | 2.9 |
| 8 | 20 |
| 9 | >1000 |
| 10 | 41 |
| 11 | 84 |
| 12 | 29 |
| 13 | 156 |
| 14 | 150 |
| 15 | 3.3 |
| 16 | 6.8 |
| 17 | >1000 |
| 18 | 2.0 |
| 19 | 21.0 |
| 20 | 9.1 |
| 21 | >1000 |
| 22 | 2.3 |
| 23 | 16.9 |
| 26 | >1000 |
| 27 | 3.4 |
| wt ADM | 1.7 |

### 1e) Stability in Human Blood Plasma

The stability of the peptides was investigated using N-terminally 6-carboxytetramethylrhodamine-(TAM)-labeled analogues.

Fluorescently labeled ADM analogues were prepared by using solid phase peptide synthesis (SPPS) as described before (general method for peptide synthesis). Manual coupling of 6-carboxytetramethylrhodamine (TAM) to the N-terminus of the peptides was carried out with a 3-fold molar excess of the fluorescence dye, 2-(1H-benzotriazol-1-yl)-1,1,3,3-tetramethyluronium hexafluorophosphate (HBTU) and N,N-Diisopropylethylamine (DIPEA) in DMF under constant shaking for 24 h the resin as described recently (Bohme D., Beck-Sickinger A. G. ChemMedChem 2015, 10: 804-14). The identity of the peptides was confirmed by mass spectrometry with an MALDI-MS (UltraflexIII, Bruker) and an ESI-MS (HCT, Bruker). The observed masses were in correspondence to the calculated masses. Purities of all analogues was demonstrated by analytical RP-HPLC and was ≧90%.

The peptides were dissolved in 1.5 ml of human blood plasma to a concentration of 10E-5 M and incubated at 37° C. under constant shaking. Samples of 150 μl were precipitated with 300 μl Ethanol/ACN (1:1) at distinct time points for at least 1 h at −20° C. After centrifugation for 30 sec at 12000 rpm, the supernatant was transferred into Costar® Spin-X® Centrifuge Tube Filters (0.22 μm) and centrifuged for 1 h at 12000 rpm. The samples were analyzed by RP-HPLC using a Varian VariTide RPC column (particle size 6 μm; pore size 200 Å; 250×4.6 mm) with linear gradients of 0.1% TFA in water and 0.08% TFA in ACN; fluorescence emission was detected at λ=573 nm. The percentage of intact peptide was determined by peak integration. The values of peaks containing additional cleavage fragments were corrected by comparing intensities of cleavage fragments and intact peptide using MALDI-MS analysis (UltraflexIII, Bruker). The stability of the peptides was calculated with GraphPad Prism 5 (GraphPad Software) using a two phase exponential decay function for the determination of slow-decay phase half-lives (ln(2)/K_{slow}; K_{slow}: rate constant of slow part of exponential decay) (Table 3 and FIG. 3).

TABLE 3
Stability in human blood plasma

| | | HalfLife | |
|---|---|---|---|
| Example | Peptide | (Slow) | |
| TAM- | TAM[G¹⁴]ADM(14-52) | 9 | h |
| control | | | |
| TAM-18 | TAM[K¹⁴(PAM), (Dpr¹⁶, E²¹)lac]ADM(14-52) | >96 | h |
| TAM-27 | TAM[K¹⁴(PAM), (Dpr¹⁶, E²¹)lac, N_{α}-Me-K⁴⁶] | >96 | h |
| | ADM(14-52) | | |

### 1f) Granulocyte Transmigration Assay

Human umbilical venous endothelial cells of passage 2 (HUVEC, Lonza) are seeded on Transwell® filter trays (24-well plates, 6.5 mm-inserts 5 μm pore size; Costar #3421, coated with fibronectin, Sigma F-1141) at a density of 2×10⁴ cells per tray in endothelial cell medium (EBM2, Lonza CC-3156, supplemented with growth supplements, Lonza CC-4176) and incubated at 37° C. and 5% CO₂ for 36 hours. Medium is replaced with fresh complete EBM2 medium containing tumor necrosis factor-alpha (TNF-α, 0.5 nM) and cells are incubated for further 7 hours. Subsequently cells are washed in MAM medium (Medium 199 supplemented with 20% FCS and 25 mM HEPES) and after addition of the test compounds to the trays further incubated for 30 min. Thereafter trays are transferred to new plates containing MAM medium with Interleukin 8 (IL-8, 5 ng/ml). Human polymorphonuclear granulocytes (PMNs, 3.7×10⁵ cells in 50 μl) are added to the inserts. After 30 min number of transmigrated cells is determined in 500 μl medium from the wells by use of a CASY® TT cell counter (Roche Innovatis AG). Anti ICAM-1 IgG (R&D Systems, BBA4) at a concentration of 100 μg/ml serves as positive control.

Human granulocytes (PMNs) are freshly isolated from 15 ml peripheral venous EDTA blood collected from healthy volunteers after giving their informed consent. In brief: blood is layered on top of a Histopaque™ 1077/Histopaque™ 1119 (12 ml each) gradient and PMNs are being collected after centrifugation for 30 min at 2100×rcf. PMNs ar finally resuspended in MAM buffer after lysing red blood cells and several wash steps.

In a typical experiment compounds are tested in a concentration range of 1×10-9 to 1×10-6 M.

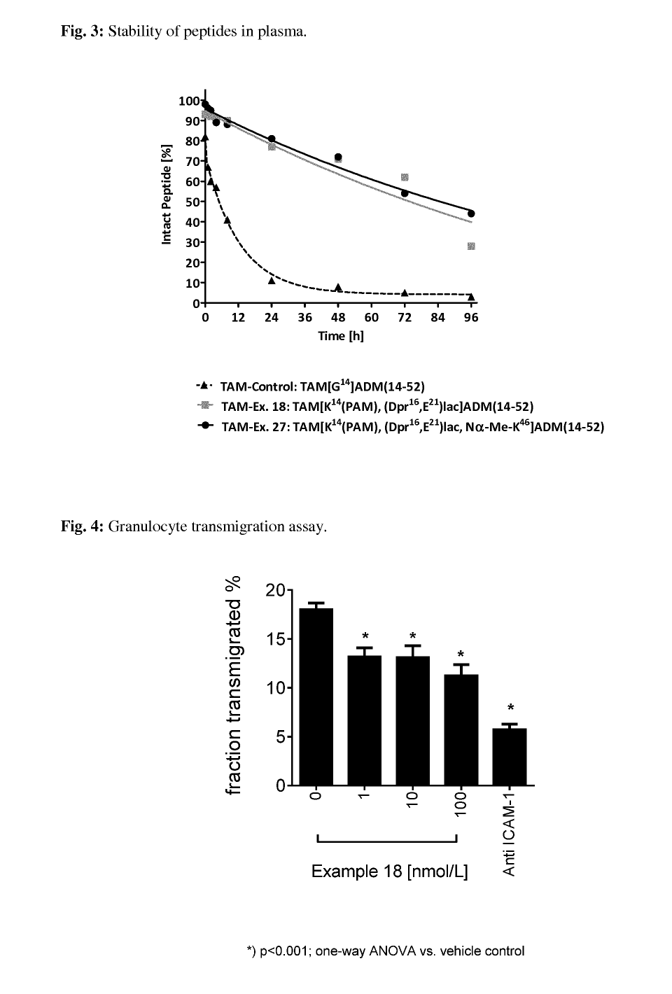

Substances according to the present invention reduced transmigration of PMNs TNF-α stimulated HUVECs at concentrations of ≧1 nmol/L [FIG. 4]

### 2. Test Descriptions (In Vivo)

### 2a) Measurement of Blood Pressure and Heart Rate in Telemetered, Normotensive Wistar Rats

The cardiovascular effects induced by compounds according to the invention are investigated in freely moving conscious female Wistar rats (body weight >200 g) by radiotelemetric measurement of blood pressure and heart rate. Briefly, the telemetric system (DSI Data Science International, MN, USA) is composed on 3 basic elements: implantable transmitters (TA11PA-C40), receivers (RA1010) and a computer-based acquisition software (Dataquest™ A.R.T. 4.1 for Windows). Rats are instrumented with pressure implants for chronic use at least 14 days prior to the experiments. The sensor catheter is tied with 4-0 suture several times to produce a stopper 0.5 cm from the tip of the catheter. During catheter implantation rats are anesthetized with pentobabital (Nembutal, Sanofi: 50 mg/kg i.p.). After shaving the abdominal skin, a midline abdominal incision is made, and the fluid-filled sensor catheter is inserted upstream into the exposed descending aorta between the iliac bifurcation and the renal arteries. The catheter is tied several times at the stopper. The tip of the telemetric catheter is located just caudal to the renal arteries and secured by tissue adhesive.

The transmitter body is affixed to the inner peritoneal wall before closure of abdomen. A two-layer closure of the abdominal incision is used, with individual suturing of the peritoneum and the muscle wall followed by closure of the outer skin. For postsurgical protection against infections and pain a single dosage of an antibiotic (Oxytetracyclin 10% R, 5.0 ml/kg s.c., beta-pharma GmbH&Co, Germany) and analgesic is injected (Rimadyl R, 5.0 ml/kg s.c., Pfizer, Germany). The hardware configuration is equipped for 24 animals. Each rat cage is positioned on top of an individual receiver platform. After activation of the implanted transmitters, an on-line data acquisition system, samples data and converts telemetric pressure signals to mm Hg. A barometric pressure reference allows for relation of absolute pressure (relative to vacuum) to ambient atmospheric pressure. Data acquisition software is predefined to sample hemodynamic data for 10-s intervals every 5 minutes. Data collection to file is started 2 hours before administration of test compounds and finished after completion of 24-h cycles. In a typical experiment test compounds are administered as bolus either subcutaneously or intravenously at a dose of 0.1 to 1000 rig/kg body weight (as referred to the peptide component).

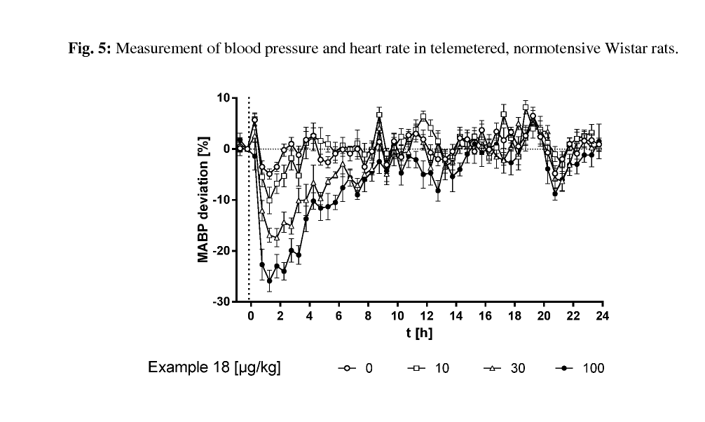

Wild type adrenomedullin (Bachem, H-2932) induces blood pressure reduction in this test with duration of ≦4 h when tested at doses of ≦300 μg/kg body weight [reference WO 2013/064508 A1]. Substances according to the present invention induced blood pressure reduction of up to 8 h at doses of ≦200 μg/kg body weight (as referred to the peptide component) [FIG. 5].

### 2b) Skin Vascular Leak Assay in Wistar Rats

An intracutaneous histamine challenge test is employed to assess the effect of compounds according to the invention on vascular barrier function in healthy animals. Male Sprague Dawley rats (body weight >200 g) are anesthetized with isoflurane (2%-3% in ambient air) and brought into supine position. The abdomen is shaved and a catheter is inserted into the femoral vein. Vehicle only (0.5 ml PBS+0.1% bovine serum albumin) or test compounds at appropriate doses are administered as i.v. bolus injections. After 15 min a second injection of 100 μl/kg 2% Evans blue (Sigma) solution is administered and immediately thereafter 100 μl of histamine solutions of appropriate concentrations (for example 0-2.5-5-10-20-40 μg/ml) are injected intracutaneously into the abdominal skin. Evans blue is a highly plasma protein bound dye and therefore used as an indicator for protein-rich fluid extravasation and vascular leakage. 30 min after this procedure the rats are sacrificed by an overdose of isoflurane and subsequent neck dislocation and the abdominal skin is excised. The wheals are excised by use of an 8 mm biopsy punch, the tissue samples are weighted and transferred to formamide for 48 h in order to extract the Evans blue. Samples are measured at 620 nM and 750 nM wavelength on a suitable photometer and Evans blue content of the samples is corrected for heme pigments according to the formula A620 (corrected)=A620−(1.426×A750+0.030) and calculated against a standard curve. [method adapted from Wang L. F., Patel M., Razavi H. M., Weicker S., Joseph M. G., McCormack D. G., Mehta S., Am. Respir Crit Care Med, 165(12), 1634-9 (2002)].

### 2c) Intra-Tracheal Instillation of LPS in Mice

An intra-tracheal challenge with lipopolysaccharide (LPS) is employed to examine the effects of compounds according to the invention on acute lung injury. Male BALB/c mice (average animal weight 20-23 g) are anesthetized with isoflurane (7%) and LPS from E. coli (e.g. serotype 055:B5; Sigma) is instilled in 100 μl saline by use of a micropipette. Typical doses of LPS used for challenge are in the range of 1 to 10 mg/kg body weight. At different time points before and after instillation test compounds are administered by the subcutaneous route. Typical doses are in the range of 1 to 300 μg/kg body weight. In this test typical time points of administration of test compounds are 15 min before or 1 h after LPS challenge. 48 hours after instillation of LPS mice are deeply anesthetized with isoflurane and sacrificed by dislocation of the neck. After cannulation of the trachea lavage of the bronchoalveolar space with 0.5 ml ice-cold saline is performed. Lungs are prepared and weighted. Cells in the bronchoalveolar lavage fluid (BALF) are counted on a cell counter (Cell Dyn 3700, Abbott). In this test lung weight as a measure for lung edema is reproducibly found to be increased by about 50% or more over sham controls 48 hours after LPS challenge. As lung weights show only very low variability in the groups, the absolute lung weight is used as parameter. The counts for white blood cells are always found to be significantly increased over control in the BALF after LPS challenge.

### 2d) Induction of Acute Lung Injury in Mini Pigs

Acute lung injury is induced in anesthetized mini pigs by use of lipopolysaccharide (LPS) or oleic acid as challenges. In detail: female Göttingen minipigs of ca. 3.5 to 5.5 kg body weight (Ellegaard, Denmark) are kept anesthetized by an continuous i.v.-infusion of Ketavet®, Dormicum® and Pancuronium® after premedication with an intramuscular injection of Ketavet®/Stresnil®. After intratracheal intubation animals are artificially ventilated using a pediatric respirator (Sulla 808V; Dräger, Germany) with an oxygen air mixture at a tidal volume of 30 to 50 ml and constant frequency of 25 min⁻¹. Arterial PaCO₂ is adjusted to about 40 mmHg by regulating the fraction of inspired oxygen (FiO₂) via the ratio of oxygen air mixture. Routinely the following cardiovascular and respiratory parameters are measured after placement of necessary probes and catheters fitted to appropriate pressure transducers and recording equipment: central venous pressure (via left jugular vein), arterial blood pressure and heart rate (BP and HR; via left carotid artery), left ventricular pressure (LVP; using a Millar catheter [FMI, Mod.:SPC-340S, REF: 800-2019-1, 4F] introduced into the left ventricle via right carotid artery), pulmonary arterial pressure (PAP; using ARROW Berman angiographic balloon catheter [REF.: AI-07134 4 Fr. 50 cm] placed into the pulmonary artery via left jugular vein), cardiac output (CO) and extravascular lung water index (EVWLI) by use of the PiCCO system (Pulsion, Germany) connected to a Pulsion 4F Thermodilution-catheter (PV2014L08N) placed into the right femoral artery. Catheters for measurement of CVP, BP, HR, LVP, and PAP are fitted to a Ponemah recording system. Arterial blood gas analysis is performed to determine the PaO₂/FiO₂. According to the American-European Consensus Conference on ARDS a PaO₂/FiO₂<300 mmHg is considered as indicative for the presence of acute lung injury. Dependent on the applied protocol duration of experiments varied between 4 and 5 hours after administration of lung injury inducing challenge. At the end of experimentation pigs are sacrificed by exsanguination and bronchoalveolar lavage fluid (BALF) is collected from lungs. Cellular content of BALF is determined by use of a blood cell counter (Cell DYN 3700).

In a typical setting acute lung injury is induced by intratracheal instillation of Lipopolysaccharide (LPS; E. coli 0111:B4; Sigma L2630) in saline into each lung via the endotracheal tube. PAP and EVWLI increased while PaO₂/FiO₂ decreased in response to the challenge. The cellular content of BALF is significantly increased.

In another protocol oleic acid (OA; Sigma-Aldrich, 01008) diluted with ethanol (1:1) is infused i.v. over 15 min at a final dose of 100 mg/kg body weight. Challenge with OA led to increase of PAP and EVLWI and decrease of PaO₂/FiO₂.

### C. EXEMPLARY EMBODIMENTS OF PHARMACEUTICAL COMPOSITIONS

The compounds according to the invention can be converted into pharmaceutical preparations in the following ways:

i.v. Solution:

A compound according to the invention is dissolved at a concentration below saturation solubility in a physiologically acceptable solvent (for example buffers of pH 4 to pH 7, isotonic sodium chloride solution, glucose solution 5% and/or PEG 400 solution 30%). The solution is sterilized by filtration and filled into sterile and pyrogen-free injection containers.

s.c. Solution:

A compound according to the invention is dissolved at a concentration below saturation solubility in a physiologically acceptable solvent (for example for example buffers of pH 4 to pH 7, isotonic sodium chloride solution, glucose solution 5% and/or PEG 400 solution 30%). The solution is sterilized by filtration and filled into sterile and pyrogen-free injection containers.

1. A compound of formula (I) 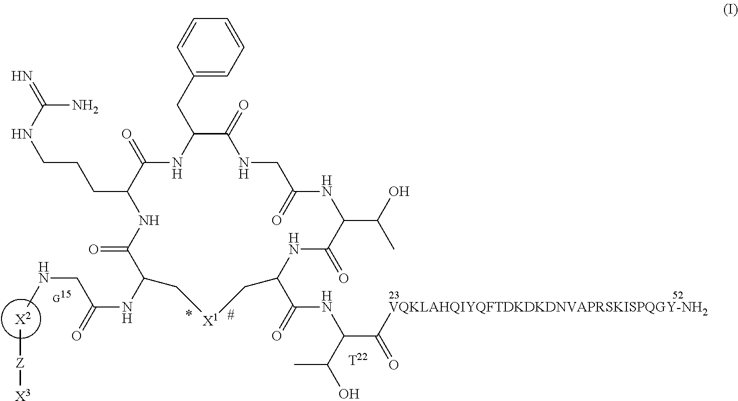
wherein X¹ is selected from the group consisting of
*—(CH₂)ₘ₁—S—^{#}, wherein m1 is 0-6; ^{#}—(CH₂)ₘ₂—S—*, wherein m2 is 0-6;
*—(CH₂)ₘ₃—^{#}, wherein m3 is 1-8;
*—(CH₂)ₘ₄—(CH₂═CH₂)—(CH₂)ₙ₁—^{#}, wherein m4 is 0-6, n1 is 0-6, with the proviso that m4+n1=0-6;
*—(CH₂)ₘ₅—(CH≡CH)—(CH₂)ₙ₂—^{#}, wherein m5 is 0-6, and n2 is 0-6, with the proviso that m5+n2=0-6;
*—(CH₂)ₘ₆—CO—NH—(CH₂)ₙ₃—^{#}, wherein m6 is 0-4, and n3 is 0-4, with the proviso that m6+n3=0-6; ^{#}—(CH₂)ₘ₇—CO—NH—(CH₂)ₙ₄—*, wherein m7 is 0-4, and n4 is 0-4, with the proviso that m7+n4=0-6;
*—SO—(CH₂)ₘ₈—^{#}, wherein m8 is 0-6; ^{#}—SO—(CH₂)ₘ₉—*, wherein m9 is 0-6;
*—SO₂—(CH₂)ₘ₁₀—^{#}, wherein m10 is 0-6; ^{#}—SO₂—(CH₂)ₘ₁₁—*, wherein m11 is 0-6;
*-5-6 membered heteroaryl-^{#};
*—O—(CH₂)ₘ₁₂—^{#}, wherein m12 is 0-6; ^{#}—O—(CH₂)ₘ₁₃—*, wherein m13 is 0-6;
*—CH₂—S—(CH₂)ₘ₁₄—^{#}, wherein m14 is 0-6; ^{#}—CH₂—S—(CH₂)ₘ₁₅—*, wherein m15 is 0-6;
*—CH₂—O—(CH₂)ₘ₁₆—^{#}, wherein m16 is 0-6; ^{#}—CH₂—O—(CH₂)ₘ₁₇—*, wherein m17 is 0-6;
*—(CH₂)ₘ₁₈—NH—CO—CH₂—NH—CO—(CH₂)ₙ₅—^{#}, wherein m18 is 0-3, and n5 is 0 or 1, with the proviso that m18+n5=0-3; ^{#}—(CH₂)ₘ₁₉—NH—CO—CH₂—NH—CO—(CH₂)ₙ₆—*, wherein m19 is 0-3, and n6 is 0 or 1, with the proviso that m19+n6=0-3;
*—(CH₂)ₘ₂₀—NH—CO—CH(CH₃)—NH—CO—(CH₂)ₙ₇—^{#}, wherein m20 is 0-3, and n7 is 0 or 1, with the proviso that m20+n7=0-3; ^{#}—(CH₂)ₘ₂₁—NH—CO—CH(CH₃)—NH—CO—(CH₂)ₙ₈—*, wherein m21 is 0-3, and n8 is 0 or 1, with the proviso that m21+n8=0-3;
*—(CH₂)ₘ₂₂—NH—CO—CH(CH₂—C(CH₃)₂)—NH—CO—(CH₂)ₙ₉—^{#}, wherein m22 is 0-3, and n9 is 0 or 1, with the proviso that m22+n9=0-3; ^{#}—(CH₂)ₘ₂₃—NH—CO—CH(CH₂—C(CH₃)₂)—NH—CO—(CH₂)ₙ₁₀—*, wherein m23 is 0-3, and n10 is 0 or 1, with the proviso that m23+n10=0-3;
*—(CH₂)ₘ₂₄—NH—CO—CH(CH(CH₃)C₂H₅)—NH—CO—(CH₂)ₙ₁₁—^{#}, wherein m24 is 0-3, and n11 is 0 or 1, with the proviso that m24+n11=0-3; ^{#}—(CH₂)ₘ₂₅—NH—CO—CH(CH(CH₃)C₂H₅)—NH—CO—(CH₂)ₙ₁₂—*, wherein m25 is 0-3, and n12 is 0 or 1, with the proviso that m25+n12=0-3;
*—(CH₂)ₘ₂₆—NH—CO—CH(CH₂(C₆H₅))—NH—CO—(CH₂)ₙ—^{#}, wherein m26 is 0-3, and n13 is 0 or 1, with the proviso that m26+n13=0-3; ^{#}—(CH₂)ₘ₂₇—NH—CO—CH(CH₂(C₆H₅))—NH—CO—(CH₂)ₙ₁₄—*, wherein m27 is 0-3, and n14 is 0 or 1, with the proviso that m27+n14=0-3;
*—(CH₂)ₘ₂₈—NH—CO—(CH₂)₃—NH—CO—(CH₂)ₙ₁₅—^{#}, wherein m28 is 0 or 1, and n15 is 0 or 1, with the proviso that m28+n15=0-1; ^{#}—(CH₂)ₘ₂₉—NH—CO—(CH₂)₃—NH—CO—(CH₂)ₙ₁₆—*, wherein m29 is 0 or 1, and n16 is 0 or 1, with the proviso that m29+n16=0-1;
*—(CH₂)ₘ₃₀—NH—CO—NH—(CH₂)ₙ₁₇—^{#}, wherein m30 is 0-5, and n17 is 0-5, with the proviso that m30+n17=0-5; ^{#}—(CH₂)ₘ₃₁—NH—CO—NH—(CH₂)ₙ₁₈—*, wherein m31 is 0-5, and n18 is 0-5, with the proviso that m31+n18=0-5;
*—(CH₂)ₘ₃₂—O—CO—NH—(CH₂)ₙ₁₉—^{#}, wherein m32 is 0-5, and n19 is 0-5, with the proviso that m32+n19=0-5; ^{#}—(CH₂)ₘ₃₃—O—CO—NH—(CH₂)ₙ₂₀—*, wherein m33 is 0-5, and n20 is 0-5, with the proviso that m33+n20=0-5;
*—(CH₂)ₘ₃₄—O—CO—O—(CH₂)ₙ₂₁—^{#}, wherein m 34 is 0-5, and n21 is 0-5, with the proviso that m34+n21=0-5;
*—(CH₂)ₘ₃₅—NH—CO—(CH₂)ₙ₂₂—NH—(CH₂)ₚ₁—, wherein m35 is 0-4, n22 is 0-4, and p1 is 0-4, with the proviso that m35+n22+p1=0-4; and
*—(CH₂)ₘ₃₆—NH—CO—(CH═CH)—CO—NH—(CH₂)ₙ₂₃—^{#}, wherein m36 is 0-2, and n23 is 0-2, with the proviso that m36+n23=0-2;
wherein * and ^{#} reflect where X¹ is bound within the ring structure; and
X² is absent, is hydrogen, or is an amino acid or amino acid sequence selected from the group consisting of G¹⁴, K¹⁴, F¹⁴, SEQ ID NO: 1 [Y¹RQSMNNFQGLRSF¹⁴], SEQ ID NO: 2 [R²QSMNNFQGLRSF¹⁴], SEQ ID NO: 3 [Q³SMNNFQGLRSF¹⁴], SEQ ID NO: 4 [S⁴MNNFQGLRSF¹⁴], SEQ ID NO: 5 [M⁵NNFQGLRSF¹⁴], SEQ ID NO: 6 [N⁶NFQGLRSF¹⁴], SEQ ID NO: 7 [N⁷FQGLRSF¹⁴], SEQ ID NO: 8 [F⁸QGLRSF¹⁴], SEQ ID NO: 9 [Q⁹GLRSF¹⁴], SEQ ID NO: 10 [G¹⁰LRSF¹⁴], SEQ ID NO: 11 [L¹¹RSF¹⁴], SEQ ID NO: 12 [R¹²SF¹⁴], and SEQ ID NO: 13 [S¹³F¹⁴], which is covalently linked by an amide bond to the N-terminal G¹⁵ of the amino acid sequence of formula (I), wherein any amino acid of X² may optionally be replaced by a natural or unnatural amino acid;
wherein A is L-Alanine; R is L-Arginine; N is L-Asparagine; D is L-Aspartic acid; Q is L-Glutamine; G is L-Glycine; H is L-Histidine; I is L-Isoleucine; L is L-Leucine; K is L-Lysine; M is L-Methionine; F is L-Phenylalanine; P is L-Proline; S is L-Serine; T is L-Threonine; Y is L-Tyrosine; V is L-Valine;
wherein the numbering of amino acids in formula (I) and in the definition of X² refers to the corresponding human ADM sequence;
X³ is absent or is a heterologous moiety which is covalently linked to the N-terminus or to a functional group of the side chain of any amino acid of X², to the N-terminus of G¹⁵ or to Z;
Z is absent or is a cleavable linker covalently bound between the N terminus of any amino acid of X² or of G¹⁵ and X³ or between a functional group of the side chain of any amino acid of X² and X³
wherein if X³ is absent, then
Z is also absent and X² is hydrogen or is an amino acid or amino acid sequence as defined above;
wherein if X³ is a heterologous moiety, then
X² is absent or is an amino acid or amino acid sequence as defined above; Z is absent or is a cleavable linker covalently bound between the N terminus of any amino acid of X² or of G¹⁵ and X³ or between a functional group of the side chain of any amino acid of X² and X³;
or a physiologically acceptable salt, a solvate or a solvate of a salt thereof.

2. The compound of formula (I) as claimed in claim 1, wherein X¹ is selected from the group consisting of
*—(CH₂)ₘ₁—S—^{#}, wherein m1 is 0-6; ^{#}—(CH₂)ₘ₂—S—*, wherein m2 is 0-6;
*—(CH₂)ₘ₃—^{#}, wherein m3 is 1-8;
*—(CH₂)ₘ₆—CO—NH—(CH₂)ₙ₃—^{#}, wherein m6 is 0-4, and n3 is 0-4, with the proviso that m6+n3=0-6; ^{#}—(CH₂)ₘ₇—CO—NH—(CH₂)ₙ₄—*, wherein m7 is 0-4, and n4 is 0-4, with the proviso that m7+n4=0-6;
X² is G¹⁴ or K¹⁴, which is covalently linked by an amide bond to the N-terminal G¹⁵ of the compound of formula (I);
X³ is absent or is a heterologous moiety which is covalently linked to the N-terminus of G¹⁴ or K¹⁴ or to a functional group of the side chain of K¹⁴, or to Z;
Z is absent or is a cleavable linker covalently bound between the N terminus of G¹⁴ or K¹⁴ and X³, or between a functional group of the side chain of K¹⁴ and X³;
wherein if X³ is absent, then Z is also absent;
wherein if X³ is a heterologous moiety, then Z is absent or is a cleavable linker covalently bound between the N terminus of G¹⁴ or K¹⁴ and X³, or between a functional group of the side chain of K¹⁴ and X³;
or a physiologically acceptable salt, a solvate or a solvate of a salt thereof.

3. The compound of formula (I) as claimed in claim 1, wherein X³ is a heterologous moiety selected from the group consisting of a polymer, a Fc, a FcRn binding ligand, albumin and an albumin-binding ligand;
or a physiologically acceptable salt, a solvate or a solvate of a salt thereof.

4. The compound of formula (I) as claimed in claim 3, wherein X³ is a polymer and the polymer is selected from the group consisting of linear or branched C₃-C₁₀₀ carboxylic acids, preferably C₄-C₃₀ carboxylic acids, optionally substituted with halo, hydroxy, alkoxy, amino, alkylamino, dialkylamino, sulfate, or phosphate, and which may be saturated, or mono- or di-unsaturated, a PEG moiety, a PPG moiety, a PAS moiety and a HES moiety;
or a physiologically acceptable salt, a solvate or a solvate of a salt thereof.

5. The compound of formula (I) as claimed in claim 4, wherein the carboxylic acid is selected from the group consisting of arachidic acid, arachidonic acid, behenic acid, capric acid, caproic acid, caprylic acid, ceroplastic acid, cerotic acid, docosahexaenoic acid, eicosapentaenoic acid, elaidic acid, enanthic acid, erucic acid, geddic acid, henatriacontylic acid, heneicosylic acid, heptacosylic acid, hexatriacontylic acid, lacceroic acid, lauric acid, lignoceric acid, linoelaidic acid, linoleic acid, margaric acid, melissic acid, montanic acid, myristic acid, myristoleic acid, nonacosylic acid, nonadecylic acid, oleic acid, palmitic acid, palmitoleic acid, pantothenic acid, pelargonic acid, pentacosylic acid, pentadecylic acid, psyllic acid, sapienic acid, stearic acid, tricosylic acid, tridecylic acid, undecylic acid, vaccenic acid, valeric acid, α-linolenic acid and derivatives thereof;
or a physiologically acceptable salt, a solvate or a solvate of a salt thereof.

6. The compound of formula (I) as claimed in claim 1, wherein Z is absent;
or a physiologically acceptable salt, a solvate or a solvate of a salt thereof.

7. The compound of formula (I) as claimed in claim 1, wherein Z is a cleavable linker;
or a physiologically acceptable salt, a solvate or a solvate of a salt thereof.

8. The compound of formula (I) as claimed claim 1, wherein the compound is further modified by N-methylation of at least one amide bond;
or a physiologically acceptable salt, a solvate or a solvate of a salt thereof.

9. The compound of formula (I) as claimed in claim 1, wherein
X¹ is selected from the group consisting of *—(CH₂)ₘ₁—S—^{#}, wherein m1 is 0-4; ^{#}—(CH₂)ₘ₂—S—*, wherein m2 is 0-4; *—(CH₂)ₘ₆—CO—NH—(CH₂)ₙ₃—^{#}, wherein m6 is 0-4, and n3 is 0-4, with the proviso that m6+n3=0-6;
X² is G¹⁴ or K¹⁴, which is covalently linked by an amide bond to the N-terminal G¹⁵ of the compound of formula (I);
X³ is absent or is a heterologous moiety which is covalently linked to the N-terminus of G¹⁴ or K¹⁴ or to a functional group of the side chain of K¹⁴, or to Z;
Z is absent or is a cleavable linker covalently bound between the N terminus of G¹⁴ or K¹⁴ and X³, or between a functional group of the side chain of K¹⁴ and X³;
wherein if X³ is absent, then Z is also absent;
wherein if X³ is a heterologous moiety, then Z is absent or is a cleavable linker covalently bound between the N terminus of G¹⁴ or K¹⁴ and X³, or between a functional group of the side chain of K¹⁴ and X³;
or a physiologically acceptable salt, a solvate or a solvate of a salt thereof.

10. A compound as claimed in claim 1 for use in a method for the treatment and/or prevention of cardiovascular, edematous and/or inflammatory disorders.

11. The compound as claimed in claim 1 for use in a method for the treatment and/or prevention of heart failure, chronic heart failure, worsening heart failure, acute heart failure, acute decompensated heart failure, diastolic and systolic (congestive) heart failure, coronary heart disease, ischemic and/or hemorrhagic stroke, hypertension, pulmonary hypertension, peripheral arterial occlusive disease, pre-eclampsia, chronic obstructive pulmonary disease, asthma, acute and/or chronic pulmonary edema, allergic alveolitis and/or pneumonitis due to inhaled organic dust and particles of fungal, actinomycetic or other origin, and/or acute chemical bronchitis, acute and/or chronic chemical pulmonary edema, neurogenic pulmonary edema, acute and/or chronic pulmonary manifestations due to radiation, acute and/or chronic interstitial lung disorders, acute lung injury/acute respiratory distress syndrome (ALI/ARDS) in adult or child including newborn, ALI/ARDS secondary to pneumonia and sepsis, aspiration pneumonia and ALI/ARDS secondary to aspiration, ALI/ARDS secondary to smoke gas inhalation, transfusion-related acute lung injury (TRALI), ALI/ARDS and/or acute pulmonary insufficiency following surgery, trauma and/or burns, and/or ventilator induced lung injury (VILI), lung injury following meconium aspiration, pulmonary fibrosis, mountain sickness, chronic kidney diseases, glomerulonephritis, acute kidney injury, cardiorenal syndrome, lymphedema, inflammatory bowel disease, sepsis, septic shock, systemic inflammatory response syndrome (SIRS) of non-infectious origin, anaphylactic shock, inflammatory bowel disease, urticaria and/or edematous ocular disorders or ocular disorders associated with disturbed vascular function.

12. A medicament comprising a compound as claimed in claim 1 in combination with an inert nontoxic pharmaceutically suitable excipient.

13. A medicament comprising a compound as claimed in claim 1 in combination with a further active ingredient selected from the group consisting of ACE inhibitors, angiotensin receptor antagonists, beta-2 receptor agonists, phosphodiesterase (PDE) inhibitors, glucocorticoid receptor agonists, diuretics, recombinant angiotensin converting enzyme-2, acetylsalicylic acid, natriuretic peptides and derivatives thereof, and neprilysin inhibitors.

14. The medicament as claimed in claim 12 for the treatment and/or prevention of cardiovascular, edematous and/or inflammatory disorders.

15. Method for the treatment and/or prophylaxis of cardiovascular, edematous and/or inflammatory disorders in humans or animals using an effective amount of at least one compound as claimed in claim 1, or a medicament comprising the at least one compound in combination with an inert nontoxic pharmaceutically suitable excipient.

16. The compound as claimed in claims 11, wherein the use is a method for the treatment and/or prevention of age-related macular degeneration (AMD) or diabetic retinopathy.

17. The compound as claimed in claim 16, wherein the use is a method for the treatment and/or prevention of diabetic macula edema (DME), subretinal edema or intraretinal edema.


SEQUENCE LISTING (27 sequences)

SEQ ID NO: 1 - protein, 39 aa, Artificial Sequence
  [G14, (E16,K21)lac]ADM(14-52) peptide
GGERFGTKTVQKLAHQIYQFTDKDKDNVAPRSKISPQGY

SEQ ID NO: 2 - protein, 39 aa, Artificial Sequence
  [G14, (K16,E21)lac]ADM(14-52) peptide
GGKRFGTETVQKLAHQIYQFTDKDKDNVAPRSKISPQGY

SEQ ID NO: 3 - protein, 39 aa, Artificial Sequence
  [G14, (Dpr16, D21)lac]ADM(14-52) peptide
  Xaa (3)..(3): N-beta-4-methyltrityl-L-Diaminopropionic acid (Dpr)
GGXRFGTDTVQKLAHQIYQFTDKDKDNVAPRSKISPQGY

SEQ ID NO: 4 - protein, 39 aa, Artificial Sequence
  [G14, (D16, Dab21)lac]ADM(14-52) peptide
  Xaa (8)..(8): 2,4-Diaminobutyric acid (Dbu)
GGDRFGTXTVQKLAHQIYQFTDKDKDNVAPRSKISPQGY

SEQ ID NO: 5 - protein, 39 aa, Artificial Sequence; identical to SEQ ID NO: 3
  [G14, (Dab16, D21)lac]ADM(14-52) peptide
  Xaa (3)..(3): 2,4-Diaminobutyric acid (Dbu)

SEQ ID NO: 6 - protein, 39 aa, Artificial Sequence
  [G14, (E16, Dpr21)lac]ADM(14-52) peptide
  Xaa (8)..(8): N-beta-4-methyltrityl-L-Diaminopropionic acid (Dpr)
GGERFGTXTVQKLAHQIYQFTDKDKDNVAPRSKISPQGY

SEQ ID NO: 7 - protein, 39 aa, Artificial Sequence
  [G14, (Dpr16, E21)lac]ADM(14-52) peptide
  Xaa (3)..(3): N-beta-4-methyltrityl-L-Diaminopropionic acid (Dpr)
GGXRFGTETVQKLAHQIYQFTDKDKDNVAPRSKISPQGY

SEQ ID NO: 8 - protein, 39 aa, Artificial Sequence; identical to SEQ ID NO: 4
  [G14, (D16, Orn21)lac]ADM(14-52) peptide
  Xaa (8)..(8): Ornithine (Orn)

SEQ ID NO: 9 - protein, 39 aa, Artificial Sequence; identical to SEQ ID NO: 3
  [G14, (Orn16, D21)lac]ADM(14-52) peptide
  Xaa (3)..(3): Ornithine (Orn)

SEQ ID NO: 10 - protein, 39 aa, Artificial Sequence; identical to SEQ ID NO: 6
  [G14, (E16, Dab21)lac]ADM(14-52) peptide
  Xaa (8)..(8): 2,4-Diaminobutyric acid (Dbu)

SEQ ID NO: 11 - protein, 39 aa, Artificial Sequence; identical to SEQ ID NO: 7
  [G14, (Dab16, E21)lac]ADM(14-52) peptide
  Xaa (3)..(3): 2,4-Diaminobutyric acid (Dbu)

SEQ ID NO: 12 - protein, 39 aa, Artificial Sequence
  [K14(PAM), (E16,K21)lac]ADM(14-52) peptide
  Lys (1)..(1): Lys is palmitoylated lysine
KGERFGTKTVQKLAHQIYQFTDKDKDNVAPRSKISPQGY

SEQ ID NO: 13 - protein, 39 aa, Artificial Sequence
  [K14(PAM), (K16,E21)lac]ADM(14-52) peptide
  Lys (1)..(1): Lys is palmitoylated lysine
KGKRFGTETVQKLAHQIYQFTDKDKDNVAPRSKISPQGY

SEQ ID NO: 14 - protein, 39 aa, Artificial Sequence; identical to SEQ ID NO: 12
  [K14(PAM), (E16, K21)lac, N-Me-I47]ADM(14-52) peptide
  Lys (1)..(1): Palmitoylated lysine
  Ile (34)..(34): N-Methylated Isoleucine (MeIle)

SEQ ID NO: 15 - protein, 39 aa, Artificial Sequence
  [G14, (C16->U21)]ADM(14-52) peptide
  Xaa (8)..(8): Xaa is U* cystathionine
GGCRFGTXTVQKLAHQIYQFTDKDKDNVAPRSKISPQGY

SEQ ID NO: 16 - protein, 39 aa, Artificial Sequence
  [G14, (U16->C21)]ADM(14-52) peptide
  Xaa (3)..(3): Xaa is U* cystathionine
GGXRFGTCTVQKLAHQIYQFTDKDKDNVAPRSKISPQGY

SEQ ID NO: 17 - protein, 39 aa, Artificial Sequence
  [G14, (U16->U21)]ADM(14-52) peptide
  Xaa (3)..(3): Xaa is U* cystathionine
  Xaa (8)..(8): Xaa is U* cystathionine
GGXRFGTXTVQKLAHQIYQFTDKDKDNVAPRSKISPQGY

SEQ ID NO: 18 - protein, 39 aa, Artificial Sequence; identical to SEQ ID NO: 13
  [K14(PAM), (Dpr16, E21)lac]ADM(14-52) peptide
  Lys (1)..(1): Palmitoylated lysine
  Xaa (3)..(3): N-beta-4-methyltrityl-L-Diaminopropionic acid (Dpr)

SEQ ID NO: 19 - protein, 39 aa, Artificial Sequence; identical to SEQ ID NO: 6
  [G14, (E16, Orn21)lac]ADM(14-52) peptide
  Xaa (8)..(8): Ornithine (Orn)

SEQ ID NO: 20 - protein, 39 aa, Artificial Sequence; identical to SEQ ID NO: 7
  [G14, (Orn16, E21)lac]ADM(14-52) peptide
  Xaa (3)..(3): Ornithine (Orn)

SEQ ID NO: 21 - protein, 39 aa, Artificial Sequence
  [G14, (K16, D21)lac]ADM(14-52) peptide
GGKRFGTDTVQKLAHQIYQFTDKDKDNVAPRSKISPQGY

SEQ ID NO: 22 - protein, 39 aa, Artificial Sequence
  [G14, (D16, K21)lac]ADM(14-52) peptide
GGDRFGTKTVQKLAHQIYQFTDKDKDNVAPRSKISPQGY

SEQ ID NO: 23 - protein, 39 aa, Artificial Sequence; identical to SEQ ID NO: 15
  PAM[G14, (C16->U21)]ADM(14-52) peptide
  Gly (1)..(1): Palmitoylated glycine
  Xaa (8)..(8): Xaa is U* cystathionine

SEQ ID NO: 24 - protein, 39 aa, Artificial Sequence
  PAM[K14, (C16->U21)]ADM(14-52) peptide
  Lys (1)..(1): Palmitoylated lysine
  Xaa (8)..(8): Xaa is U* cystathionine
KGCRFGTXTVQKLAHQIYQFTDKDKDNVAPRSKISPQGY

SEQ ID NO: 25 - protein, 39 aa, Artificial Sequence; identical to SEQ ID NO: 24
  [K14(PAM), (C16->U21)]ADM(14-52) peptide
  Lys (1)..(1): Palmitoylated lysine
  DISULFID (8)..(8): Xaa is U* cystathionine

SEQ ID NO: 26 - protein, 39 aa, Artificial Sequence; identical to SEQ ID NO: 4
  [G14, (D16, Dpr21)lac]ADM(14-52) peptide
  Xaa (8)..(8): N-beta-4-methyltrityl-L-Diaminopropionic acid (Dpr)

SEQ ID NO: 27 - protein, 39 aa, Artificial Sequence
  [K14(PAM), (Dpr16, E21)lac, N-Me-K46]ADM(14-52) peptide
  Lys (1)..(1): Palmitoylated lysine
  Xaa (3)..(3): N-beta-4-methyltrityl-L-Diaminopropionic acid (Dpr)
  Lys (33)..(33): N-Methylated Lysine (MeLys)
KGXRFGTETVQKLAHQIYQFTDKDKDNVAPRSKISPQGY